SEQUENCE LISTING (34 sequences)

SEQ ID NO: 1 - DNA, 2758 bp, Homo sapiens
ctgggaggcgggcgcacatcgatggcgtcaccttctggcgccgccggttggtttccctggcaactggagcaatcagagcaccgcagccagggagatgctggcgagacgacagcgagatcctctccaggccctgcggcgccgcaatcaggagctgaagcaacaggttgatagtttgctttctgagagccaactgaaagaagctctagaacccaataaaagacaacatatttatcaaagatgtatccagttaaagcaggcaatagatgaaaataaaaatgctcttcaaaaattaagcaaagctgatgaatctgcacctgttgcaaactataatcagagaaaagaagaggagcatactcttttggacaagcttacccaacaactgcagggccttgctgtgacaataagcagagaaaatataactgaagttggggcacctactgaagaagaggaagaaagtgaaagtgaagatagtgaagacagtggtggggaggaagaagatgcagaggaggaagaggaagagaaagaggaaaatgaatctcacaaatggtcaaccggtgaagaatacatcgctgttggagattttactgctcagcaagttggagatcttacatttaagaaaggggaaattctccttgtaattgaaaaaaaacctgatggttggtggatagctaaggatgccaaaggaaatgaaggtcttgttcccagaacctacctagagccttatagtgaagaagaagaaggccaagagtcaagtgaagagggcagtgaagaagatgtagaggcggtggatgaaacagcagatggagcagaagttaagcaaagaactgatccccactggagtgctgttcagaaagcgatttcagaggcgggcatcttctgtcttgttaatcatgtctcgttttgctacctaatagttctgatgcgaaataggatggagactgtggaagacaccaatggatctgaaacagggttcagggcatggaatgtacagagcagaggacgtatatttctggtttctaagcctgtgctccaacagataaacactgttgatgtgttaactacgatgggagctattcctgcagggttcaggccttccacgctctcacagcttctggaggaagggaatcaatttcgagcaaattacttcttacaaccagagctcatgccttcacaactggccttcagagatctgatgtgggatgctacagaaggcactattaggtcgagaccaagtcgtatttcattgattctgacattatggagctgtaaaatgattcctcttccaggaatgagcatacaggttctcagcagacatgtacgcctctgtctatttgatggtaataaggttctgagcaacattcatacagtcagagccacatggcaacctaaaaagcccaaaacatggaccttttctccccaggttactcgcatcttaccatgtttgcttgatggtgattgctttatcaggtctaattctgcatctccagatcttggaatattatttgaacttggaatttcttatattcgcaattcaactggtgaaagaggagagttaagctgtggctgggtgtttcttaaactttttgatgccagtggagttcctattccagcaaaaacttatgagcttttcttgaatggtggtactccttatgaaaaaggtattgaagtggacccttcaatatccagaagagcacacggcagtgttttctaccagattatgacaatgagaaggcagcctcaacttctagtgaaactgagatccttgaacagaagatcaagaaatgtactaagtctactgccagaaacattaattggaaatatgtgttctattcacttgttgatattttatcgacaaattcttggagatgtgctcctgaaagacaggatgagcttgcaaagtactgatttaattagccatcccatgctggccaccttccccatgctcttggagcagcctgatgtgatggatgctctcaggagttcgtgggctggaaaagaaagcacattaaaaagatcagagaagagagacaaagagttcctgaagtccacgtttctcctggtttaccatgactgcgtgctcccacttctccactccacacgcctacccccattcaggtgggcagaagaagagactgagactgcacggtggaaagttatcactgacttccttaagcaaaaccaagaaaaccagggcgccctccaagctctgctgtcaccagacggagttcatgaaccttttgacctttcagagcagacctatgacttcttgggtgaaatgagaaagaatgcagtgtgacagtggcagcctctagccctcagcttcccacggaatcagatggatcctccacgattacgtgaataaaatgatggaaccaaaaatcactgtcactttacaacttaggttttactcttttctttctacagaccatatttttaaagaaatgtttatacaataatttaaatattttttaaaaccataaaataaatttttataaggaatactgttatatctaaatttaaacagtatttattttttcaaaaacagctacttaagttaatggtatagatttctataaaagcaagattttgtcaaaaactaaatttatgattattcaagaaagtgaaaaaaacaacctacagaatgggaaaacatatttgcaaatcatctaactgataaaggtctagtatccaaaatatttaaatttatgagtgttaataaaatttatcttgttcaatgaagaggaagttaaaaaaaaaa

SEQ ID NO: 2 - DNA, 2755 bp, Homo sapiens
ctgggaggcgggcgcacatcgatggcgtcaccttctggcgccgccggttggtttccctggcaactggagcaatcagagcaccgcagccagggagatgctggcgagacgacagcgagatcctctccaggccctgcggcgccgcaatcaggagctgaagcaacaggttgatagtttgctttctgagagccaactgaaagaagctctagaacccaataaaagacaacatatttatcaaagatgtatccagttaaagcaggcaatagatgaaaataaaaatgctcttcaaaaattaagcaaagctgatgaatctgcacctgttgcaaactataatcagagaaaagaagaggagcatactcttttggacaagcttacccaacaactgcagggccttgctgtgacaataagcagagaaaatataactgaagttggggcacctactgaagaagaggaagaaagtgaaagtgaagatagtgaagacagtggtggggaggaagaagatgcagaggaggaagaggaagagaaagaggaaaatgaatctcacaaatggtcaaccggtgaagaatacatcgctgttggagattttactgctcagcaagttggagatcttacatttaagaaaggggaaattctccttgtaattgaaaaaaaacctgatggttggtggatagctaaggatgccaaaggaaatgaaggtcttgttcccagaacctacctagagccttatagtgaagaagaagaaggccaagagtcaagtgaagagggcagtgaagaagatgtagaggcggtggatgaaacagcagatggagcagaagttaagcaaagaactgatccccactggagtgctgttcagaaagcgatttcagaggcgggcatcttctgtcttgttaatcatgtctcgttttgctacctaatagttctgatgcgaaataggatggagactgtggaagacaccaatggatctgaaacagggttcagggcatggaatgtacagagcagaggacgtatatttctggtttctaagcctgtgctccaaataaacactgttgatgtgttaactacgatgggagctattcctgcagggttcaggccttccacgctctcacagcttctggaggaagggaatcaatttcgagcaaattacttcttacaaccagagctcatgccttcacaactggccttcagagatctgatgtgggatgctacagaaggcactattaggtcgagaccaagtcgtatttcattgattctgacattatggagctgtaaaatgattcctcttccaggaatgagcatacaggttctcagcagacatgtacgcctctgtctatttgatggtaataaggttctgagcaacattcatacagtcagagccacatggcaacctaaaaagcccaaaacatggaccttttctccccaggttactcgcatcttaccatgtttgcttgatggtgattgctttatcaggtctaattctgcatctccagatcttggaatattatttgaacttggaatttcttatattcgcaattcaactggtgaaagaggagagttaagctgtggctgggtgtttcttaaactttttgatgccagtggagttcctattccagcaaaaacttatgagcttttcttgaatggtggtactccttatgaaaaaggtattgaagtggacccttcaatatccagaagagcacacggcagtgttttctaccagattatgacaatgagaaggcagcctcaacttctagtgaaactgagatccttgaacagaagatcaagaaatgtactaagtctactgccagaaacattaattggaaatatgtgttctattcacttgttgatattttatcgacaaattcttggagatgtgctcctgaaagacaggatgagcttgcaaagtactgatttaattagccatcccatgctggccaccttccccatgctcttggagcagcctgatgtgatggatgctctcaggagttcgtgggctggaaaagaaagcacattaaaaagatcagagaagagagacaaagagttcctgaagtccacgtttctcctggtttaccatgactgcgtgctcccacttctccactccacacgcctacccccattcaggtgggcagaagaagagactgagactgcacggtggaaagttatcactgacttccttaagcaaaaccaagaaaaccagggcgccctccaagctctgctgtcaccagacggagttcatgaaccttttgacctttcagagcagacctatgacttcttgggtgaaatgagaaagaatgcagtgtgacagtggcagcctctagccctcagcttcccacggaatcagatggatcctccacgattacgtgaataaaatgatggaaccaaaaatcactgtcactttacaacttaggttttactcttttctttctacagaccatatttttaaagaaatgtttatacaataatttaaatattttttaaaaccataaaataaatttttataaggaatactgttatatctaaatttaaacagtatttattttttcaaaaacagctacttaagttaatggtatagatttctataaaagcaagattttgtcaaaaactaaatttatgattattcaagaaagtgaaaaaaacaacctacagaatgggaaaacatatttgcaaatcatctaactgataaaggtctagtatccaaaatatttaaatttatgagtgttaataaaatttatcttgttcaatgaagaggaagttaaaaaaaaaa

SEQ ID NO: 3 - DNA, 2590 bp, Homo sapiens
ctgggaggcgggcgcacatcgatggcgtcaccttctggcgccgccggttggtttccctggcaactggagcaatcagagcaccgcagccagggagatgctggcgagacgacagcgagatcctctccaggccctgcggcgccgcaatcaggagctgaagcaacaggttgatagtttgctttctgagagccaactgaaagaagctctagaacccaataaaagacaacatatttatcaaagatgtatccagttaaagcaggcaatagatgaaaataaaaatgctcttcaaaaattaagcaaagctgatgaatctgcacctgttgcaaactataatcagagaaaagaagaggagcatactcttttggacaagcttacccaacaactgcagggccttgctgtgacaataagcagagaaaatataactgaagttggggcacctactgaagaagaggaagaaagtgaaagtgaagatagtgaagacagtggtggggaggaagaagatgcagaggaggaagaggaagagaaagaggaaaatgaatctcacaaatggtcaaccggtgaagaatacatcgctgttggagattttactgctcagcaagttggagatcttacatttaagaaaggggaaattctccttgtaattgaaaaaaaacctgatggttggtggatagctaaggatgccaaaggaaatgaaggtcttgttcccagaacctacctagagccttatagtgaagaagaagaaggccaagagtcaagtgaagagggcagtgaagaagatgtagaggcggtggatgaaacagcagatggagcagaagttaagcaaagaactgatccccactggagtgctgttcagaaagcgatttcagagcagataaacactgttgatgtgttaactacgatgggagctattcctgcagggttcaggccttccacgctctcacagcttctggaggaagggaatcaatttcgagcaaattacttcttacaaccagagctcatgccttcacaactggccttcagagatctgatgtgggatgctacagaaggcactattaggtcgagaccaagtcgtatttcattgattctgacattatggagctgtaaaatgattcctcttccaggaatgagcatacaggttctcagcagacatgtacgcctctgtctatttgatggtaataaggttctgagcaacattcatacagtcagagccacatggcaacctaaaaagcccaaaacatggaccttttctccccaggttactcgcatcttaccatgtttgcttgatggtgattgctttatcaggtctaattctgcatctccagatcttggaatattatttgaacttggaatttcttatattcgcaattcaactggtgaaagaggagagttaagctgtggctgggtgtttcttaaactttttgatgccagtggagttcctattccagcaaaaacttatgagcttttcttgaatggtggtactccttatgaaaaaggtattgaagtggacccttcaatatccagaagagcacacggcagtgttttctaccagattatgacaatgagaaggcagcctcaacttctagtgaaactgagatccttgaacagaagatcaagaaatgtactaagtctactgccagaaacattaattggaaatatgtgttctattcacttgttgatattttatcgacaaattcttggagatgtgctcctgaaagacaggatgagcttgcaaagtactgatttaattagccatcccatgctggccaccttccccatgctcttggagcagcctgatgtgatggatgctctcaggagttcgtgggctggaaaagaaagcacattaaaaagatcagagaagagagacaaagagttcctgaagtccacgtttctcctggtttaccatgactgcgtgctcccacttctccactccacacgcctacccccattcaggtgggcagaagaagagactgagactgcacggtggaaagttatcactgacttccttaagcaaaaccaagaaaaccagggcgccctccaagctctgctgtcaccagacggagttcatgaaccttttgacctttcagagcagacctatgacttcttgggtgaaatgagaaagaatgcagtgtgacagtggcagcctctagccctcagcttcccacggaatcagatggatcctccacgattacgtgaataaaatgatggaaccaaaaatcactgtcactttacaacttaggttttactcttttctttctacagaccatatttttaaagaaatgtttatacaataatttaaatattttttaaaaccataaaataaatttttataaggaatactgttatatctaaatttaaacagtatttattttttcaaaaacagctacttaagttaatggtatagatttctataaaagcaagattttgtcaaaaactaaatttatgattattcaagaaagtgaaaaaaacaacctacagaatgggaaaacatatttgcaaatcatctaactgataaaggtctagtatccaaaatatttaaatttatgagtgttaataaaatttatcttgttcaatgaagaggaagttaaaaaaaaaa

SEQ ID NO: 4 - DNA, 2401 bp, Homo sapiens
ctgggaggcgggcgcacatcgatggcgtcaccttctggcgccgccggttggtttccctggcaactggagcaatcagagcaccgcagccagggagatgctggcgagacgacagcgagatcctctccaggccctgcggcgccgcaatcaggagctgaagcaacaggttgatagtttgctttctgagagccaactgaaagaagctctagaacccaataaaagacaacatatttatcaaagagttggggcacctactgaagaagaggaagaaagtgaaagtgaagatagtgaagacagtggtggggaggaagaagatgcagaggaggaagaggaagagaaagaggaaaatgaatctcacaaatggtcaaccggtgaagaatacatcgctgttggagattttactgctcagcaagttggagatcttacatttaagaaaggggaaattctccttgtaattgaaaaaaaacctgatggttggtggatagctaaggatgccaaaggaaatgaaggtcttgttcccagaacctacctagagccttatagtgaagaagaagaaggccaagagtcaagtgaagagggcagtgaagaagatgtagaggcggtggatgaaacagcagatggagcagaagttaagcaaagaactgatccccactggagtgctgttcagaaagcgatttcagagataaacactgttgatgtgttaactacgatgggagctattcctgcagggttcaggccttccacgctctcacagcttctggaggaagggaatcaatttcgagcaaattacttcttacaaccagagctcatgccttcacaactggccttcagagatctgatgtgggatgctacagaaggcactattaggtcgagaccaagtcgtatttcattgattctgacattatggagctgtaaaatgattcctcttccaggaatgagcatacaggttctcagcagacatgtacgcctctgtctatttgatggtaataaggttctgagcaacattcatacagtcagagccacatggcaacctaaaaagcccaaaacatggaccttttctccccaggttactcgcatcttaccatgtttgcttgatggtgattgctttatcaggtctaattctgcatctccagatcttggaatattatttgaacttggaatttcttatattcgcaattcaactggtgaaagaggagagttaagctgtggctgggtgtttcttaaactttttgatgccagtggagttcctattccagcaaaaacttatgagcttttcttgaatggtggtactccttatgaaaaaggtattgaagtggacccttcaatatccagaagagcacacggcagtgttttctaccagattatgacaatgagaaggcagcctcaacttctagtgaaactgagatccttgaacagaagatcaagaaatgtactaagtctactgccagaaacattaattggaaatatgtgttctattcacttgttgatattttatcgacaaattcttggagatgtgctcctgaaagacaggatgagcttgcaaagtactgatttaattagccatcccatgctggccaccttccccatgctcttggagcagcctgatgtgatggatgctctcaggagttcgtgggctggaaaagaaagcacattaaaaagatcagagaagagagacaaagagttcctgaagtccacgtttctcctggtttaccatgactgcgtgctcccacttctccactccacacgcctacccccattcaggtgggcagaagaagagactgagactgcacggtggaaagttatcactgacttccttaagcaaaaccaagaaaaccagggcgccctccaagctctgctgtcaccagacggagttcatgaaccttttgacctttcagagcagacctatgacttcttgggtgaaatgagaaagaatgcagtgtgacagtggcagcctctagccctcagcttcccacggaatcagatggatcctccacgattacgtgaataaaatgatggaaccaaaaatcactgtcactttacaacttaggttttactcttttctttctacagaccatatttttaaagaaatgtttatacaataatttaaatattttttaaaaccataaaataaatttttataaggaatactgttatatctaaatttaaacagtatttattttttcaaaaacagctacttaagttaatggtatagatttctataaaagcaagattttgtcaaaaactaaatttatgattattcaagaaagtgaaaaaaacaacctacagaatgggaaaacatatttgcaaatcatctaactgataaaggtctagtatccaaaatatttaaatttatgagtgttaataaaatttatcttgttcaatgaagaggaagttaaaaaaaaaa

SEQ ID NO: 5 - protein, 733 aa, Homo sapiens
MLARRQRDPLQALRRRNQELKQQVDSLLSESQLKEALEPNKRQHIYQRCIQLKQAIDENKNALQKLSKADESAPVANYNQRKEEEHTLLDKLTQQLQGLAVTISRENITEVGAPTEEEEESESEDSEDSGGEEEDAEEEEEEKEENESHKWSTGEEYIAVGDFTAQQVGDLTFKKGEILLVIEKKPDGWWIAKDAKGNEGLVPRTYLEPYSEEEEGQESSEEGSEEDVEAVDETADGAEVKQRTDPHWSAVQKAISEAGIFCLVNHVSFCYLIVLMRNRMETVEDTNGSETGFRAWNVQSRGRIFLVSKPVLQQINTVDVLTTMGAIPAGFRPSTLSQLLEEGNQFRANYFLQPELMPSQLAFRDLMWDATEGTIRSRPSRISLILTLWSCKMIPLPGMSIQVLSRHVRLCLFDGNKVLSNIHTVRATWQPKKPKTWTFSPQVTRILPCLLDGDCFIRSNSASPDLGILFELGISYIRNSTGERGELSCGWVFLKLFDASGVPIPAKTYELFLNGGTPYEKGIEVDPSISRRAHGSVFYQIMTMRRQPQLLVKLRSLNRRSRNVLSLLPETLIGNMCSIHLLIFYRQILGDVLLKDRMSLQSTDLISHPMLATFPMLLEQPDVMDALRSSWAGKESTLKRSEKRDKEFLKSTFLLVYHDCVLPLLHSTRLPPFRWAEEETETARWKVITDFLKQNQENQGALQALLSPDGVHEPFDLSEQTYDFLGEMRKNAV

SEQ ID NO: 6 - protein, 732 aa, Homo sapiens
MLARRQRDPLQALRRRNQELKQQVDSLLSESQLKEALEPNKRQHIYQRCIQLKQAIDENKNALQKLSKADESAPVANYNQRKEEEHTLLDKLTQQLQGLAVTISRENITEVGAPTEEEEESESEDSEDSGGEEEDAEEEEEEKEENESHKWSTGEEYIAVGDFTAQQVGDLTFKKGEILLVIEKKPDGWWIAKDAKGNEGLVPRTYLEPYSEEEEGQESSEEGSEEDVEAVDETADGAEVKQRTDPHWSAVQKAISEAGIFCLVNHVSFCYLIVLMRNRMETVEDTNGSETGFRAWNVQSRGRIFLVSKPVLQINTVDVLTTMGAIPAGFRPSTLSQLLEEGNQFRANYFLQPELMPSQLAFRDLMWDATEGTIRSRPSRISLILTLWSCKMIPLPGMSIQVLSRHVRLCLFDGNKVLSNIHTVRATWQPKKPKTWTFSPQVTRILPCLLDGDCFIRSNSASPDLGILFELGISYIRNSTGERGELSCGWVFLKLFDASGVPIPAKTYELFLNGGTPYEKGIEVDPSISRRAHGSVFYQIMTMRRQPQLLVKLRSLNRRSRNVLSLLPETLIGNMCSIHLLIFYRQILGDVLLKDRMSLQSTDLISHPMLATFPMLLEQPDVMDALRSSWAGKESTLKRSEKRDKEFLKSTFLLVYHDCVLPLLHSTRLPPFRWAEEETETARWKVITDFLKQNQENQGALQALLSPDGVHEPFDLSEQTYDFLGEMRKNAV

SEQ ID NO: 7 - protein, 677 aa, Homo sapiens
MLARRQRDPLQALRRRNQELKQQVDSLLSESQLKEALEPNKRQHIYQRCIQLKQAIDENKNALQKLSKADESAPVANYNQRKEEEHTLLDKLTQQLQGLAVTISRENITEVGAPTEEEEESESEDSEDSGGEEEDAEEEEEEKEENESHKWSTGEEYIAVGDFTAQQVGDLTFKKGEILLVIEKKPDGWWIAKDAKGNEGLVPRTYLEPYSEEEEGQESSEEGSEEDVEAVDETADGAEVKQRTDPHWSAVQKAISEQINTVDVLTTMGAIPAGFRPSTLSQLLEEGNQFRANYFLQPELMPSQLAFRDLMWDATEGTIRSRPSRISLILTLWSCKMIPLPGMSIQVLSRHVRLCLFDGNKVLSNIHTVRATWQPKKPKTWTFSPQVTRILPCLLDGDCFIRSNSASPDLGILFELGISYIRNSTGERGELSCGWVFLKLFDASGVPIPAKTYELFLNGGTPYEKGIEVDPSISRRAHGSVFYQIMTMRRQPQLLVKLRSLNRRSRNVLSLLPETLIGNMCSIHLLIFYRQILGDVLLKDRMSLQSTDLISHPMLATFPMLLEQPDVMDALRSSWAGKESTLKRSEKRDKEFLKSTFLLVYHDCVLPLLHSTRLPPFRWAEEETETARWKVITDFLKQNQENQGALQALLSPDGVHEPFDLSEQTYDFLGEMRKNAV

SEQ ID NO: 8 - protein, 614 aa, Homo sapiens
MLARRQRDPLQALRRRNQELKQQVDSLLSESQLKEALEPNKRQHIYQRVGAPTEEEEESESEDSEDSGGEEEDAEEEEEEKEENESHKWSTGEEYIAVGDFTAQQVGDLTFKKGEILLVIEKKPDGWWIAKDAKGNEGLVPRTYLEPYSEEEEGQESSEEGSEEDVEAVDETADGAEVKQRTDPHWSAVQKAISEINTVDVLTTMGAIPAGFRPSTLSQLLEEGNQFRANYFLQPELMPSQLAFRDLMWDATEGTIRSRPSRISLILTLWSCKMIPLPGMSIQVLSRHVRLCLFDGNKVLSNIHTVRATWQPKKPKTWTFSPQVTRILPCLLDGDCFIRSNSASPDLGILFELGISYIRNSTGERGELSCGWVFLKLFDASGVPIPAKTYELFLNGGTPYEKGIEVDPSISRRAHGSVFYQIMTMRRQPQLLVKLRSLNRRSRNVLSLLPETLIGNMCSIHLLIFYRQILGDVLLKDRMSLQSTDLISHPMLATFPMLLEQPDVMDALRSSWAGKESTLKRSEKRDKEFLKSTFLLVYHDCVLPLLHSTRLPPFRWAEEETETARWKVITDFLKQNQENQGALQALLSPDGVHEPFDLSEQTYDFLGEMRKNAV

SEQ ID NO: 9 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 10 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 11 - protein, 352 aa, Homo sapiens
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 12 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 13 - protein, 317 aa, Homo sapiens [sequence omitted]

SEQ ID NO: 14 - protein, 396 aa, Homo sapiens
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVSTQAEQLRRQLTPYAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLRGNTEGLQKSLAELGGHLDQQVEEFRRRVEPYGENFNKALVQQMEQLRQKLGPHAGDVEGHLSFLEKDLRDKVNSFFSTFKEKESQDKTLSLPELEQQQEQQQEQQQEQVQMLAPLES

SEQ ID NO: 15 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 16 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 17 - DNA, 23 bp, Homo sapiens
gcgcctgtgcatcagtggtaata

SEQ ID NO: 18 - DNA, 20 bp, Homo sapiens
actcaccgaggctgccgaat

SEQ ID NO: 19 - DNA, 20 bp, Homo sapiens
ccacccaggaagagccatgc

SEQ ID NO: 20 - DNA, 19 bp, Homo sapiens
gtgccgcactcaccagctc

SEQ ID NO: 21 - DNA, 20 bp, Homo sapiens
atgggggtaggcgtgtggag

SEQ ID NO: 22 - DNA, 21 bp, Homo sapiens
tgcacagcctgaccatacctg

SEQ ID NO: 23 - DNA, 25 bp, Homo sapiens
acagcaccaatgctaatactcacct

SEQ ID NO: 24 - DNA, 22 bp, Homo sapiens
caggtcaatcagtgccaagcca

SEQ ID NO: 25 - DNA, 23 bp, Homo sapiens
agagatgacaagaccttgaggca

SEQ ID NO: 26 - DNA, 19 bp, Homo sapiens
caggggctgacagcatggc

SEQ ID NO: 27 - DNA, 21 bp, Homo sapiens
tgtgggggtggtgttttccca

SEQ ID NO: 28 - DNA, 20 bp, Homo sapiens
tggcgagacgacagcgagat

SEQ ID NO: 29 - DNA, 24 bp, Homo sapiens
gctgcaacagtatattgcatgctc

SEQ ID NO: 30 - DNA, 22 bp, Homo sapiens
catttgtgctcatgcagcctgg

SEQ ID NO: 31 - DNA, 25 bp, Homo sapiens
tgtgcaactaactcaggattccact

SEQ ID NO: 32 - DNA, 21 bp, Homo sapiens
gctggcgctgctgtctctaac

SEQ ID NO: 33 - DNA, 20 bp, Homo sapiens
gcgtggctctgctctgcatt

SEQ ID NO: 34 - DNA, 25 bp, Homo sapiens
gctcacctcgtttctcatctccttg

(19) United States
(12) Patent Application Publication
(10) Pub. No.: US 2011/0195903 A1
(43) Pub. Date: Aug. 11, 2011

(54) METHODS AND COMPOSITIONS FOR DETECTING RECESSIVE FAMILIAL FSGS AND USES THEREOF

(76) Inventor: Chaker N. Adra, Boston, MA (US)

(21) Appl. No.: 12/969,564

(22) Filed: Dec. 15, 2010

Related U.S. Application Data
(60) Provisional application No. 61/286,782, filed on Dec. 15, 2009.
(60) Provisional application No. 61/340,295, filed on Mar. 13, 2010.
(60) Provisional application No. 61/315,888, filed on Mar. 19, 2010.

(51) Int. Cl. A61K 38/02; C12Q 1/68; G01N 33/68; A61K 48/00; A61K 31/56; A61P 13/12; G01N 33/559; G01N 27/447

(52) U.S. Cl. 514/15.4; 435/6.1; 436/86; 436/501; 435/7.92; 514/44 R; 514/169; 204/461

(57) ABSTRACT

Described herein are genomic and proteomic biomarkers for the diagnosis of FSGS. Methods for diagnosing FSGS or a predisposition to develop FSGS using the described biomarkers are also provided. Further provided are methods for choosing a course of treatment or administering treatment based on a diagnosis of FSGS using the disclosed biomarkers.

### RELATED APPLICATIONS

This application claims the benefit under 35 U.S.C. §119 of U.S. provisional applications U.S. Ser. No. 61/286,782, filed Dec. 15, 2009; U.S. Ser. No. 61/340,295, filed Mar. 13, 2010; and U.S. Ser. No. 61/315,888, filed Mar. 19, 2010; the entire disclosure of each of which is incorporated herein by reference.

### GOVERNMENT SUPPORT

This invention was made with U.S. Government support under grants DK54931, 1S10RR023004, and P30DK079130, awarded by the National Institutes of Health. The U.S. Government has certain rights in this invention.

### FIELD OF THE INVENTION

The invention relates to methods and compositions for detecting and treating Familial Focal and Segmental Glomerulosclerosis (FSGS).

### BACKGROUND OF THE INVENTION

End Stage Renal Disease (ESRD) is a significant cause of morbidity in Saudi Arabia (SA) and worldwide. The main data source on renal disease incidence in SA is available on End Stage Renal Disease published annually by the Saudi Center for Organ Transplantation (SCOT: www.scot.org.sa/). In the latest annual report from SCOT; 11,168 ESRD patients were reported on dialysis and 2,350 of these patients presented with ESRD in the year 2008. The reported average ESRD incidence rate is 150 patients per million per year in the period from 1999 to 2008. In 2008 alone there were 1300 mortalities amongst ESRD patients (12% of the total reported ESRD cases). These numbers reveals the incidence rates for ESRD, while the incidence of Chronic Kidney Failure (CRF) remains largely undetermined. Obtaining more accurate statistics about CRF has been challenging because of factors such as individual's latency of recognizing symptoms, the need to refer patients from rural health facilities to central specialized hospitals to perform advanced analyses, such quantitative proteinuria analysis and kidney biopsies, and individual's avoidance-attitude towards the relatively intrusive kidney biopsy procedures (19-21). These factors are also behind difficulties of obtaining meticulous and extensive clinical and histopathological reports from CRF patients in early stages of the disease.

Focal and segmental glomerulosclerosis (FSGS) is a form of chronic kidney disease that manifests in different clinical and histopathological patterns in affected patients. Some patients “with FSGS” respond to steroids, some do not; some patients present with nephrotic syndrome (NS), others with mild proteinuria; some present in childhood, some as adults. FSGS phenotype can be familial, primary (idiopathic), or secondary to a multitude of pathological processes affecting the kidney, including tubulointerstitial diseases such as nephronophthisis.

Focal segmental glomerulosclerosis (FSGS) is a cause of nephrotic syndrome in children and adolescents, as well as an important cause of kidney failure in adults. It is also known as “focal glomerular sclerosis” or “focal nodular glomerulosclerosis”.

The individual components of the condition refer to the appearance of the kidney tissue on biopsy: focal—only some of the glomeruli are involved (as opposed to diffuse), segmental—only part of an entire glomerulus is involved (as opposed to global), glomerulosclerosis—refers to scarring of the glomerulus (a part of the nephron (the functional unit of the kidney)). The glomerulosclerosis is usually indicated by heavy PAS staining and findings of IgM and C3 in sclerotic segment.

Depending on the cause, FSGS is broadly classified as either i) primary, when no underlying cause is found; usually presents as nephrotic syndrome; or ii) secondary, when an underlying cause is identified; usually presents with kidney failure and proteinuria. Proteinuria refers to the presence of an excess of serum protein in the urine of a subject, for example, of about 5-20 mg/dL (trace), about 20-30 mg/dL (mild), about 30-100 100 mg/dL, about 100-300 mg/dL, about 300-2000 mg/dL, or more than 2000 mg/dL. FSGS is actually a heterogeneous group including numerous causes such as a) infections such as HIV (known as HIV-Associated Nephropathy); b) toxins and drugs such as heroin and pamidronate; c) familial forms; or d) secondary to nephron loss and hyperfiltration, such as with chronic pyelonephritis and reflux, morbid obesity, diabetes mellitus.

The glomerulus function is to provide blood filtration. Dis-functional podocytes and slit diaphragm lead to higher proteinuria levels. The diagnosis of FSGS is based on kidney biopsy and requires the presence of areas with glomerular sclerosis and tuft collapse that are both focal and segmental.

There are currently several known genetic causes of the hereditary forms of FSGS. Mutations in the following genes have been associated with FSGS: i) alpha-Act-4 (ACTN4, which encodes alpha-actinin-4 cross-links bundles of actin filaments and is present in the podocyte—mutations in this protein associated with FSGS result in increased affinity for actin binding, formation of intracellular aggregates, and decreased protein half-life); ii) NPHS2 (mutations in the NPHS2 gene, which codes for the protein called podocin, can cause focal segmental glomerulosclerosis—this is a recessive form of FSGS); CD2AP (CD2AP—CD2 associated protein—or CMS—Cas binding protein with multiple SH3 domains—is expressed in podocytes where it interacts with fyn and synaptopodin and is involved in dynamic actin remodeling and membrane to cytoskeleton signal trafficking); and TRPC6 (TRPC6 encodes a member of the canonical family of TRP channels—this family of ion channels conduct cations in a largely non-selective manner—TRPC6 is expressed in podocytes—while TRP channels can be activated through a variety of methods, TRPC6 is known to be activated by phospholipase C stimulation—there are at least 6 mutations in this channel, located throughout the channel—at least one of these mutations, P112Q, leads to increased intracellular calcium influx). Mutations in the PCLE1 gene also have been associated with FSGS (PCLE1 is involved in differentiation).

Progress in understanding the genetic basis of inherited glomerular diseases is helping to compose meaningful classification of these pathologies and increase accuracy of diagnosis. Utilizing advanced technologies in the study of inherited kidney diseases facilitate not only distinguishing disease entities of somewhat mixed phenotypic and histopathologic patterns, but also drawing conclusions from analysis performed on a small number of individuals. Such technologies as whole genome genotyping coupled with whole exome capture followed by massive sequencing can increase the efficiency, accuracy, and speed of diagnosis.

FSGS and other disorders of the kidney can be complex with overlapping phenotypes.

### SUMMARY OF THE INVENTION

Some aspects of this invention relate to methods and genetic and proteomic biomarkers for the diagnosis of FSGS or a risk of developing FSGS. One or more of the genetic and proteomic biomarkers disclosed herein are risk factors that can be used to assist in the diagnosis of FSGS or a risk of developing FSGS.

Some aspects of this invention provide a method comprising determining a genotype or haplotype of the Nephrocystin-1 (NPHP1) genomic locus in a subject, and, if both alleles of the NPHP1 genomic locus comprise a loss of function mutation, identifying the subject as having or being predisposed to develop Focal Segmental Glomerulosclerosis (FSGS). In some embodiments, the method further comprises obtaining a biological sample from the subject for genotyping or haplotyping. In some embodiments, the method further comprises performing an assay on the nucleic acid sample to determine the genotype or haplotype of the NPHP1 genomic locus. In some embodiments, the subject is homozygous for a loss of function mutation at the NPHP1 genomic locus. In some embodiments, the subject is an adult. In some embodiments, the subject is not diagnosed or indicated to have nephronophthisis (NPH). In some embodiments, the mutation is a deletion of a genomic region coding for the NPHP1 protein or a fragment thereof. In some embodiments, the subject belongs to a family in which at least one member is or has been diagnosed with or affected by FSGS. In some embodiments, the subject belongs to a family in which at least one member is or has been diagnosed with or affected by FSGS but no member of which has been diagnosed or affected with NPH. In some embodiments, the method further comprises choosing a course of treatment and/or administering a treatment appropriate for FSGS to the subject in order to prevent or delay development of FSGS in the subject. In some embodiments, the subject comprises a deletion of one or more of the following genes: MALL, NPHP1, LOC151009, LIMS3, RGPD8, RGPD6, or RGPD 5. In some embodiments, the subject was identified as having proteinurea.

Some aspects of this invention provide a method comprising determining the genotype and/or haplotype of the NPHP1 genomic locus in a subject from a family with a history of FSGS, comparing the genotype and/or haplotype to a genotype and/or haplotype of the NPHP1 genomic locus in a plurality of consanguineous subjects having FSGS, and comparing the genotype and/or haplotype to a genotype and/or haplotype of the NPHP1 genomic locus in a plurality of consanguineous subjects not having FSGS, wherein if the genotype and/or haplotype of the subject comprises a loss of function mutation at the NPHP1 genomic locus that is shared among the subjects having FSGS, then the subject is indicated to be predisposed to develop FSGS, or if the genotype and/or haplotype of the subject does not comprise a loss of function mutation at the NPHP1 genomic locus, then the subject is indicated to not be predisposed to develop FSGS. In some embodiments, the method further comprised choosing a course of treatment or administering a treatment appropriate for FSGS to the subject predisposed to develop FSGS to prevent or delay development of FSGS in the subject. In some embodiments, the NPHP1 loss of function mutation is a deletion of the NPHP1 gene. In some embodiments, the subject is identified as having a deletion of one or more of the following genes: MALL, NPHP1, LOC151009, LIMS3, RGPD8, RGPD6, or RGPD 5. In some embodiments, the subject is identified as being homozygous for a deletion of one or more of the following genes: MALL, NPHP1, LOC151009, LIMS3, RGPD8, RGPD6, or RGPD 5. In some embodiments, the determining is before the onset of FSGS in the subject.

Some aspects of this invention provide a method comprising determining the genotype and/or haplotype of the NPHP1 genomic locus of a male subject, determining the genotype and/or haplotype of the NPHP1 genomic locus a female subject, and if both genotypes and/or haplotypes share a loss of function mutation at the NPHP1 genomic locus, identifying their potential progeny as being at an increased risk to have a genotype predisposing the carrier to develop FSGS. In some embodiments, the male subject and/or the female subject are from a family with a history of FSGS. In some embodiments, the male subject and/or the female subject has a deletion of one or more of the following genes: MALL, NPHP1, LOC151009, LIMS3, RGPD8, RGPD6, or RGPD 5.

Some aspects of this invention provide a method comprising (a) analyzing proteins contained in a serum sample obtained from a subject from a family in which at least one member was or is affected by FSGS, wherein the subject has a deletion of both alleles of one or more of the following genes: MALL, NPHP1, LOC151009, LIMS3, RGPD8, RGPD6, or RGPD 5; (b) comparing the proteins contained in the serum sample of (a) to proteins contained in a serum sample from a consanguineous subject that does not have a deletion of both alleles of one or more of the following genes: MALL, NPHP1, LOC151009, LIMS3, RGPD8, RGPD6, or RGPD 5, wherein if a protein is contained in the serum sample obtained from the subject having the deletion but not in the serum sample from the subject not having the deletion, then the protein is identified as an FSGS-specific serum protein. In some embodiments, the method further comprises obtaining the serum sample of (a) and/or of (b). In some embodiments, the method further comprises performing an analytical assay to determine the levels of the proteins in the serum sample under (a) and/or (b). In some embodiments, the analytical assay is a 2D protein gel electrophoresis analysis.

Some aspects of this invention provide a method comprising obtaining a biological sample from a subject, determining the level of a first FSGS-specific serum protein in the sample, comparing the level of the first protein to a reference level indicative of an average risk for FSGS, identifying the subject as having or being predisposed to FSGS, if the level of the first protein is statistically different than the reference level.

Some aspects of this invention provide a method comprising obtaining a biological sample from a subject, determining the level of a first FSGS-specific serum protein in the sample, comparing the level of the first protein to a reference level indicative of an elevated risk for FSGS, identifying the subject as having or being predisposed to FSGS if the level of the first protein is statistically similar to the reference level. In some embodiments, the biological sample is a serum sample. In some embodiments, the first protein is a protein shown in FIG. 12, 13, 14, or 15. In some embodiments, the first protein is a member of a complement and/or coagulation cascade, a transport protein, or a zinc finger protein. In some embodiments, the first protein is selected from a group of proteins including alpha 1 antitrypsin, beta-2 glycoprotein, alpha-1 microglobulin, transthyretin, or a precursor thereof, apolipoprotein E, or a precursor thereof, apolipoprotein A IV, or a precursor thereof, serotransferrin, or a precursor thereof, and Vitamin D binding protein, or a precursor thereof. In some embodiments, the level of the first protein is detected using an antibody assay. In some embodiments, the level of the first protein is detected using an ELISA or a Western blot.

Some aspects of this invention provide a method comprising obtaining a biological sample containing genomic DNA from a subject, determining the genotype of the NPHP1 genomic locus in the subject, and, if the genome comprises a mutation of the NPHP1 genomic locus, the subject is indicated to have or to be predisposed to develop a renal disease. In some embodiments, the renal disease is FSGS. In some embodiments, the subject is an adult. In some embodiments, the subject is not diagnosed or indicated to have Nephronophthisis (NPH). In some embodiments, the mutation is a deletion of a genomic region coding for NPHP1 protein or a fragment thereof. In some embodiments, the subject belongs to a family with a history of FSGS. In some embodiments, the subject belongs to a family with a history of FSGS but no history of NPH. In some embodiments, the method further comprises administering healthcare to the subject.

Some aspects of this invention provide a method, comprising determining the genotype and/or haplotype of a subject having FSGS, determining the genotype and/or haplotype of a consanguineous subject having FSGS, determining the genotype and/or haplotype of a consanguineous subject not having FSGS, comparing said genotype and/or haplotype of the subject with the genotype and/or haplotype of the consanguineous subject having FSGS and/or with the genotype and/or haplotype of the consanguineous subject not having FSGS, and identifying a genomic segment that is shared without recombination between subjects having FSGS but not between subjects not having FSGS as a segment identical by descent that is implicated in FSGS. In some embodiments, the segment identical by descent implicated in FSGS is a segment on chromosome 2. In some embodiments, the segment identical by descent implicated in FSGS is a segment located between 2p11.2 and 2q21.3 on chromosome 2. In some embodiments, the segment identical by descent is a segment located between 2q12.2 and 2q14.2 on chromosome 2.

Some aspects of this invention provide a method, comprising determining the genotype and/or haplotype of a subject from a family with a history of FSGS, comparing the genotype and/or haplotype to a genotype and/or haplotype obtained from a plurality of consanguineous subjects having FSGS, and comparing the genotype and/or haplotype to a genotype and/or haplotype obtained from a plurality of consanguineous subjects not having FSGS, wherein, if the genotype and/or haplotype of the subject comprises a segment identical by descent that is shared among the subjects having FSGS, then the subject is indicated to be predisposed to develop FSGS, or, if the genotype and/or haplotype of the subject does not comprise a segment identical by descent that is shared among the subjects having FSGS, then the subject is indicated to not be predisposed to develop FSGS. In some embodiments, the method further comprises administering healthcare to the subject predisposed to develop FSGS to prevent or delay development of FSGS in the subject. In some embodiments, the segment identical by descent is a segment on chromosome 2. In some embodiments, the segment identical by descent is a segment located between 2p11.2 and 2q21.3 on chromosome 2. In some embodiments, the segment identical by descent is a segment located between 2q12.2 and 2q14.2 on chromosome 2. In some embodiments, the segment identical by descent comprises the NPHP1 gene.

In some embodiments, a method is provided, comprising determining the genotype and/or haplotype of a male subject, determining the genotype and/or haplotype of a female subject, and if both genotypes and/or haplotypes share a segment identical by descent implicated in FSGS, identifying their potential progeny as being at risk to have a genotype predisposing the carrier to develop FSGS. In some embodiments, the male subject and/or the female subject are from a family with a history of FSGS. In some embodiments, the segment identical by descent is a segment on chromosome 2. In some embodiments, the segment identical by descent is a segment located between 2p11.2 and 2q21.3 on chromosome 2. In some embodiments, the segment identical by descent is a segment located between 2q12.2 and 2q14.2 on chromosome 2. In some embodiments, if both genotypes and/or haplotypes indicate a deletion or loss of function mutation in the NPHP1 gene, then the potential progeny is identified as being at risk to have a genotype predisposing the carrier to develop FSGS.

Some aspects of this invention provide a method for diagnosing kidney disease, the method comprising assessing whether a subject not having nephronophthisis has a deletion of or loss of function mutation in the NPHP1 gene, wherein, if the subject has an NPHP1 deletion or loss of function deletion, then the subject is indicated to have FSGS or to be at an elevated risk of developing FSGS. In some embodiments, the method further comprises administering healthcare to the subject to delay the onset or ameliorate the diagnosed FSGS.

Some aspects of this invention provide a method for diagnosing kidney disease in a subject, the method comprising assaying NPHP1 in the subject, for example, by determining whether the subject has a deletion or a loss-of-function mutation in the NPHP1 locus or by determining an expression level of NPHP1 in the subject; if the subject is determined to have a deletion, loss of function mutation, or decreased expression of NPHP1, then determining whether the subject exhibits any clinical symptoms of nephronophthisis, for example, any symptoms of a ciliopathy in the kidney or in another organ; and, if the subject does not exhibit any symptoms of a ciliopathy, then the subject is indicated to have FSGS or an elevated risk of developing FSGS. In some embodiments, the method further comprises administering to a subject indicated to have FSGS an appropriate treatment for FSGS. In some embodiments, the method further comprises not administering to the subject a treatment appropriate for nephronophthisis.

Some aspects of this invention provide a method of identifying a FSGS specific serum protein, the method comprising obtaining a serum sample from a subject having FSGS, performing an analysis of the proteins contained in the serum sample, comparing the proteins contained in the serum sample to the proteins contained in a serum sample from a subject not having FSGS, wherein if a protein is contained in the serum sample obtained from the subject having FSGS but not in the serum sample from the subject not having FSGS, then the protein is identified as an FSGS-specific serum protein. In some embodiments, the analysis is via a 2D protein gel electrophoresis analysis, western blot, ELISA, or protein array assay.

Some aspects of this invention provide a method of diagnosing or assisting in the diagnosis of FSGS, the method comprising obtaining a biological sample from a subject, determining the level of a first protein in the sample, comparing the level of the first protein to a reference level indicative of an average risk for FSGS, identifying the subject as having or being predisposed to FSGS if the level of the first protein is statistically different than the reference level. Some aspects of this invention provide a method of diagnosing or assisting in the diagnosis of FSGS, the method comprising obtaining a biological sample from a subject, determining the level of a first protein in the sample, comparing the level of the first protein to a reference level indicative of an elevated risk for FSGS, identifying the subject as having or being predisposed to FSGS if the level of the first protein is statistically similar to the reference level. In some embodiments, the biological sample is a serum sample. In some embodiments, the first protein is a protein shown in FIG. 12, 13, 14, or 15. In some embodiments, the first protein is a member of a complement and/or coagulation cascade, a transport protein, or a zinc finger protein. In some embodiments, the first protein is selected from a group of proteins including alpha 1 antitrypsin, beta-2 glycoprotein, alpha-1 microglobulin, transthyretin, or a precursor thereof, apolipoprotein E, or a precursors thereof, apolipoprotein A IV, or a precursor thereof, serotransferrin, or a precursor thereof, and Vitamin D binding protein, or a precursor thereof. In some embodiments, the level of the first protein is detected using an antibody assay. In some embodiments, the level of the first protein is detected using an ELISA or a Western blot. In some embodiments, a subject at an elevated risk of or a subject predisposed to developing FSGS is a subject the risk to develop FSGS of which is increased by about 10-25%, by about 25-50%, by about 50-75%, by about 75-100%, by about 100-1000%, or by about 1000-10000%, as compared to an average subject. In some embodiments, a subject at an elevated risk of or a subject predisposed to developing FSGS is a subject the risk to develop FSGS of which is about 5-10%, about 10-20%, about 20-30%, about 30-40%, about 40-50%, about 50-60%, about 60-70%, about 70-80%, about 80-90%, about 90-95%, about 95-99%, about 99%, or about 100%.

Some aspects of this invention are related to the genetic characterization of consanguineous individuals in unrelated Saudi Arabian families with familial FSGS. Affected individuals in the families presented with renal failure and clinical and histological features consistent with focal segmental glomerulosclerosis. Since FSGS patients may present atypical radiological findings, making the clinical diagnosis of the genetic syndrome difficult, whole-genome single-nucleotide polymorphism analysis followed by state of the art sequence capture and exome sequencing on genomic DNA samples from these families was performed for genetic characterization. These analyses facilitated accurate diagnosis after isolation of a homozygosity run of ˜2 Mb in two of the families. This homozygous run falls between rs6754115 (genomic position 109,328,776) and rs17464100 (genomic position 111,284,252), which includes the NPHP1 genomic locus, and is identical in affected subjects from two of the unrelated families. This provides evidence that this deletion is widely spread in the families' geographical regions, and implies its significant involvement in the development of chronic kidney failure in Saudi Arabia. Some aspects of this invention provide methods for performing diagnostic genetic screening for this NPHP allele in renal failure patients and outline an assay for this purpose.

Some aspects of this invention provide genetic and protein biomarkers for the diagnosis of FSGS. Some aspects of this invention provide methods for diagnosing FSGS in a subject based on a deletion or loss-of-function mutation in the NPHP1 gene in the subject. Some aspects of this invention provide methods for diagnosing FSGS in a subject based on an elevated level of alpha 1 antitrypsin, beta-2 glycoprotein, alpha-1 microglobulin, transthyretin, or a precursor thereof, apolipoprotein E, or a precursor thereof, apolipoprotein A IV, or a precursor thereof, serotransferrin, or a precursor thereof, and/or Vitamin D binding protein in a subject. In some embodiments, the elevated level is an elevated serum level. In some embodiments, the elevated level is an elevated expression level, for example, as measured by an elevated protein level in a cell, tissue, or body fluid of the subject.

Some aspects of this invention provide methods for choosing a course of treatment of a subject based on assessment of a diagnostic biomarker provided herein. For example, some embodiments provide methods of diagnosing FSGS in a patient and choosing a course of treatment appropriate for FSGS.

### BRIEF DESCRIPTION OF THE DRAWINGS

FIG. 1: Homozygosity Mapping in Two Consanguineous Families. Pedigrees for a) family 12 (FAM012) and b) family 1 (FAM001) are shown. Squares are males and circles are females and pedigree identifier is shown in the bottom of the figure. Arrow heads point to affected subjects which share the homozygous runs that are indicated as a horizontal, shaded bar on the chromosome view in the right side for each pedigree.

FIG. 2: Histopathology. Kidney biopsy findings in an affected member of FAM012. a) Masson's trichrome stain representation of a glomerulus showing Glomerular tuft segmental sclerosis in the superior half; segments in the inferior half display mesangium and capillary walls and lumens with conserved architecture. b) Direct immunofluorescence showing focal staining for C3 and IgM in the sclerosed glomerular segments. c) and d) Electron micrographs from kidney biopsy revealing areas of extensive foot process effacement (arrowheads) and glomerular basement membrane wrinkling, changes consistent with focal segmental glomerulosclerosis (FSGS).

FIG. 3: Homozygous run shared in FAM012 and FAM001. The homozygous run localize between rs6754115 (genomic position 109,328,776) and rs17464100 (genomic position 111,284,252) and the critical homozygous interval is ˜2 Mb.

FIG. 4A: Exome capture and sequence data from the NPHP1/MALL locus. In RKH-5, there is zero sequencing coverage inside the deletion, while in KFH-41 there was an average coverage of 74× (black arrowheads). Both RKH-5 and KFH-41 had sequencing coverage in the region outside the deletion (arrowheads). (This figure was generated using Integrated Genomic View: www.broadinstitute.gov/igv/).

FIG. 4B: Schematic of primer binding sites used in the deletion PCR assay. NPHP1 deletion was detected in patient RKH005 but not in patient KFH041.

FIGS. 5-7: Analysis results for three families. In FIG. 5, candidate regions are shown in chromosome 5 and 2 (see narrower shaded bars on the chromosomes). Similarly, candidate regions on chromosomes, 15, 7, 5, 4, 3, and 2 are shown in FIG. 6. Similarly, a candidate region is shown on chromosome 2 in FIG. 7.

FIG. 8: Detailed map of chromosome 2, including an area of overlap between regions identified in different families.

FIG. 9: Families that were analyzed.

FIG. 10: Examples of 2D gels and results for test and control samples.

FIG. 11: Examples of protein profiling using 2D gels.

FIGS. 12 and 13: 21 protein spots that can be used according to aspects of the invention (e.g., as markers for FSGS). It should be appreciated that the presence of one or more of these markers in a sample may be evaluated using a 2D analysis, and/or using a ligand (e.g., an antibody) that specifically detects the presence of one or more of these markers in a patient sample.

FIGS. 14 and 15: Differential expression of certain protein markers. The figures also illustrate a protein of interest (see boxed spot).

FIG. 16: Hierarchical cluster analysis using the expression patterns of 21 protein spots s that are differentially expressed between normal and FSGS samples (N=Normal and FSGS=FSGS) (A). The correspondence analysis of the same dataset is shown in (B).

FIG. 17: Non-limiting technique for assaying samples for the presence of one or more FSGS-specific protein markers.

### DETAILED DESCRIPTION

In some embodiments, the invention relates to genetic and/or protein biomarkers for the diagnosis of FSGS. The markers provided herein, alone or in combination, are useful for the diagnosis of FSGS in a subject, for the diagnosis of an elevated risk or predisposition to develop FSGS in a subject, and for the recommendation of a clinical intervention (e.g., for the choice of a course of treatment) in a subject having, suspected to have, or at an elevated risk of developing FSGS.

Chronic renal failure (CRF) diseases cause chronic kidney injuries of variable histopathological patterns that are widely utilized to classify kidney diseases. For example, CRF diseases can be classified into renal glomerular disease and renal cystic ciliopathies. The question of whether these histopathological patterns are distinct diseases, or mere descriptions of kidney biopsy specimens at particular points remain the subject of continuous debate.

Focal segmental glomerulosclerosis (FSGS) is characterized by focal and segmental glomerular scaring (sclerosis) in some, but not all, glomeruli of the kidneys. FSGS may occur as a primary process resulting from a defect in glomerular podocyte function, or secondary to many chronic kidney injuries. Almost any chronic kidney injury, irrespective of cause, can lead to secondary glomerular sclerosis, which can be visualized histologically in kidney biopsies by segmental or global fibrosis of a number of glomeruli. Ultrastructural examination of kidney biopsies is essential to distinguish between processes that have podocyte injury as a root cause (primary FSGS) and processes in which podocyte injury and glomerulosclerosis appear is subsequent to other chronic injuries (secondary FSGS) (see Mistry K, Ireland J H, Ng R C, Henderson J M, et al. Novel mutations in NPHP4 in a consanguineous family with histological findings of focal segmental glomerulosclerosis. Am J Kidney Dis 2007; 50: 855-864; and Thomas D B. Focal segmental glomerulosclerosis: a morphologic diagnosis in evolution. Archives of pathology & laboratory medicine 2009; 133: 217-223). FSGS may also be inherited as a Mendelian trait (familial FSGS), which shows histopathological findings similar to those seen in primary FSGS. Studies of familial FSGS and related nephrotic syndrome have provided novel insights into the mechanisms of human kidney disease. Several genes have been identified as causative, when mutated, to the inherited form of FSGS and/or other related nephrotic syndrome and these genes encode podocyte structural and functional proteins (see Denamur E, Bocquet N, Mougenot B, Da Silva F, et al. Mother-to-child transmitted WT1 splice-site mutation is responsible for distinct glomerular diseases. J Am Soc Nephrol 1999; 10: 2219-2223; Boute N, Gribouval O, Roselli S, Benessy F, et al. NPHS2, encoding the glomerular protein podocin, is mutated in autosomal recessive steroid-resistant nephrotic syndrome. Nature genetics 2000; 24: 349-354; Kaplan J M, Kim S H, North K N, Rennke H, et al. Mutations in ACTN4, encoding alpha-actinin-4, cause familial focal segmental glomerulosclerosis. Nature genetics 2000; 24: 251-256; Winn M P, Conlon P J, Lynn K L, Farrington M K, et al. A mutation in the TRPC6 cation channel causes familial focal segmental glomerulosclerosis. Science (New York, N.Y. 2005; 308: 1801-1804; Lowik M M, Groenen P J, Pronk I, Lilien M R, et al. Focal segmental glomerulosclerosis in a patient homozygous for a CD2AP mutation. Kidney international 2007; 72: 1198-1203; Philippe A, Nevo F, Esquivel E L, Reklaityte D, et al. Nephrin mutations can cause childhood-onset steroid-resistant nephrotic syndrome. J Am Soc Nephrol 2008; 19: 1871-1878; Brown E J, Schlondorff J S, Becker D J, Tsukaguchi H, et al. Mutations in the formin gene INF2 cause focal segmental glomerulosclerosis. Nature genetics 42: 72-76; Pollak M R. The genetic basis of FSGS and steroid-resistant nephrosis. Seminars in nephrology 2003; 23: 141-146). Identifying these genes has provided significant knowledge of the pathogenesis of hereditary glomerular disease, better understanding of podocyte function, and motivation for the ongoing search for additional FSGS associated genes.

Progress in understanding the genetic basis of inherited glomerular diseases is helping to compose meaningful classification of these pathologies and increase accuracy of diagnosis. Utilizing advanced technologies in the study of inherited kidney diseases facilitate not only distinguishing disease entities of somewhat mixed phenotypic and histopathologic patterns, but also drawing conclusions from analysis performed on a small number of individuals. The exploitation of technologies such as whole genome genotyping coupled with whole exome capture followed by massive sequencing, as provided by some aspects of this invention, can increase the efficiency, accuracy, and speed of diagnosis, and help in the selection of an appropriate course of treatment.

FSGS and other disorders of the kidney can be complex with overlapping phenotypes. Some aspects of this invention provide diagnostic methods and assays that are based on performing whole-genome genetic evaluation of patients and their families. These methods and assays can be used to complement conventional clinical and histopathological diagnostic methods to achieve a more accurate diagnosis, but are also valuable when sufficient clinical and histopathological information can not be obtained. Some aspects of this invention provide an approach to diagnose patients with hereditary kidney disease with histological findings that are consistent with FSGS based on recent advances in genetic technologies. This approach allows to effectively evaluate families with inherited kidney failure to provide an accurate diagnosis and also sheds light on a possibly widely spread allelic variant in consanguineous families with kidney failure presenting with findings consistent with FSGS in Saudi Arabia.

Currently, the leading diagnostic feature of renal glomerular diseases is proteinuria. Steroid-resistant nephrotic syndrome (SRNS), which typically manifests histologically as focal segmental glomerulosclerosis (FSGS), remains one of the most intractable kidney diseases. In children it carries a 30% risk of recurrence in a kidney transplant. Multiple single-gene causes of SRNS have been identified. Recessive mutations in NPHS1 (nephrin) cause congenital nephrotic syndrome with onset by 90 days of life. Mutations of NPHS2 (podocin) cause 10-28% of all non-familial childhood SRNS cases. With very few exceptions, all monogenic forms of SRNS lead to chronic kidney disease (CKD) and are resistant to steroid treatment.

Renal cystic ciliopathies include autosomal dominant polycystic kidney disease (ADPKD), the most frequent lethal dominant disease in the United States and Europe, afflicting about 1 in 1,000 individuals, and nephronophthisis (NPHP), the most frequent genetic cause for CKD in the first three decades of life. CKD develops by at a median age of 13 years. In contrast to PKD, in patients with nephronophthisis, cysts are mostly restricted to the corticomedullary border of the kidneys, and kidney size is normal or reduced. Mutations in nine different recessive genes (NPHP1-NPHP9) have been identified as causing NPHP. It can be associated with retinal degeneration (Senior-Loken syndrome, SLSN), liver fibrosis, or cerebellar vermis aplasia (Joubert syndrome, JBTS).

In NPHP the nature of the two recessive mutations determines severity and extent of organ involvement, leading to seemingly different disorders. Within this varied genotype-phenotype correlation loss-of-function mutations cause severe, early-onset, dysplastic, multiorgan disease (Meckel-Gruber syndrome), whereas reduced function mutations cause mild, late-onset, degenerative disease with limited organ involvement (NPHP with retinal degeneration). See Hildebrandt F. Genetic kidney diseases. Lancet. 2010 Apr. 10; 375(9722): 1287-95.

In some embodiments, aspects of the invention relate to the diagnosis of FSGS. In some embodiments, aspects of this invention relate to the diagnosis of a genetic predisposition of a subject to FSGS. In some embodiments, aspects of this invention relate to the diagnosis of the likelihood of a genetic predisposition of the offspring of a subject to FSGS. In some embodiments, aspects of the invention relate to the detection of a loss of a functional NPHP1 gene in a subject as a marker for FSGS or predisposition to FSGS. In some embodiments, loss of a functional NPHP1 gene is identified as a homozygous deletion of the NPHP1 locus or a portion thereof. However, loss of a functional NPHP1 gene may be identified as a functional loss of one or both alleles of the NPHP1 gene in a subject. In some embodiments, a functional loss of an NPHP1 allele may be a deletion of all or a portion of the NPHP1 gene. In some embodiments, a functional loss of an NPHP1 allele may be due to a mutation (e.g., a frameshift, a stop codon, or other loss of function mutation) at one or more positions in the NPHP1 gene. In some embodiments, a functional loss of an NPHP1 allele may be due to an inversion or other rearrangement of the NPHP1 gene. In some embodiments, a functional loss of an NPHP1 allele may be due to an insertion or duplication at one or more positions in the NPHP1 gene. It should be appreciated that any change in the NPHP1 gene may be within one or more introns or exons of the gene. In some embodiments, a functional loss of an NPHP1 gene may be caused by a mutation that interferes with the correct splicing of one or more introns/exons of the gene. In some embodiments, a functional loss of an NPHP1 allele may be caused by a mutation, deletion, inversion, insertion, duplication or other genetic rearrangement, or a combination thereof, at one or more positions upstream or downstream of the NPHP1 gene (e.g., in a regulatory region) or within the NPHP1 that decreases or otherwise interferes with appropriate expression of the NPHP1 gene.

In some embodiments, an NPHP1 loss of function is associated with a deletion of one or more additional genes described herein (e.g., MALL, LOC151009, LIMS3, RGPD8, RGPD6, or RGPD 5). In some embodiments, the deletion encompasses NPHP1 and any one or more or all of MALL, LOC151009, LIMS3, RGPD8, RGPD6, and RGPD 5.

In some embodiments, a subject is identified as having FSGS, or being at risk for FSGS, if the subject is identified as missing both functional alleles of the NPHP1 gene. It should be appreciated that the subject may be identified as homozygous for one or more of the loss of function genetic changes described above or elsewhere herein. However, in some embodiments, the subject may be heterozygous for one or more different loss of function genetic changes at each of the alleles of the NPHP1 locus.

In some embodiments, a subject may be screened for the presence of one or more NPHP1 associated mutations by obtaining a biological sample from the subject and assaying the sample for a genomic change indicative of a loss of NPHP1 function, an abnormal (e.g., lower than normal) level of NPHP1 mRNA, an abnormal (e.g., lower than normal) level of NPHP1 protein, or any combination thereof. A biological sample may be a blood sample, a serum sample, a urine sample, a tissue biopsy, a sample of any other biological fluid or tissue, as aspects of the invention are not limited in this respect.

In some embodiments, aspects of the invention relate to genetic and/or protein markers for FSGS. In some embodiments, a subject determined to have one or more genetic markers (e.g., one or more loss of function mutations or deletions of one or more of the genes MALL, NPHP1, LOC151009, LIMS3, RGPD8, RGPD6, or RGPD 5) and/or one or more protein markers (e.g., abnormal expression of one or more of proteins Alpha 1 antitrypsin, beta-2 glycoprotein, alpha-1 microglobulin, transthyretin precursor (Prealbumin), apolipoprotein E precursor, apolipoprotein A IV precursor, serotransferrin precursor, and/or Vitamin D binding protein precursor) is identified as being at risk for FSGS. A subject identified as at risk for FSGS may be i) evaluated more carefully for additional indicia of FSGS and/or ii) treated to avoid or reduce the development or progression of FSGS. In some embodiments, one or more of the genetic markers and/or protein markers described herein may be an early sign of risk for FSGS. A subject that has one or more of these markers may be evaluated to determine whether the subject has additional markers. For example, if a subject has a mutation/deletion of a first marker gene, the subject may be further evaluated to determine whether the subject has a mutation/deletion of one or more of the other genes described herein. Similarly, if a subject is identified as having an abnormal level of expression (e.g., over expression) of one or more protein markers described herein, the subject may be further evaluated to determine whether one or more additional protein markers are over expressed. It also should be appreciated that a subject with identified as having at least one genetic marker may be evaluated for the presence of at least one protein marker, and that a subject identified as having at least one protein marker may be evaluated for the presence of at least one genetic marker. In some embodiments, subjects with one or more genetic or protein markers also may be evaluated for other physiological and/or histological signs or symptoms of FSGS as described herein. It should be appreciated that in some embodiments, a subject's degree of risk for FSGS is related to the number of markers for FSGS that are present in the subject. In some embodiments, a subject that has only one or a few markers of FSGS may be monitored more regularly for additional markers of FSGS in order to determine whether the subject's risk is increasing and/or whether the disease is progressing. From a therapeutic perspective, a subject that has at least one risk factor for FSGS (e.g., one marker described herein) may be treated with appropriate diet and or therapeutic regimen to prevent or delay the onset or progression of the disease.

It should be appreciated that aspects of the invention may be used to screen subjects that have no prior risk factors for FSGS. However, in some embodiments, subjects that have at least one family member with FSGS may be identified as candidates for screens for the presence of one or more markers described herein.

In some embodiments, aspects of this invention relate to the detection of a biomarker indicative of FSGS in a subject. In some embodiments, aspects of this invention relate to diagnosis of FSGS or a genetic predisposition to FSGS based on the detection of a biomarker in a subject. In some embodiments, the biomarker is a gene or a gene product, for example, a mutation in a gene or a genomic locus, expression, expression level or mutation of an mRNA or a protein. In some embodiments, a biological sample containing a gene or gene product of interest is obtained from a subject and a molecular detection assay is performed. Assays useful for detection of a gene, or a gene product are well known to those of skill in the art and include, for example, nucleic acid hybridization based methods, sequencing methods, genomic sequencing methods, exome sequencing methods, PCR, RT-PCR, microarray assays, SNP-assays (e.g., PCR-sequencing), Northern blot assays, protein binding assays, immunoassays, ELISA, southern blot, western blot, and many others, for example, as described in Joe Sambrook, Molecular Cloning: A Laboratory Manual, Volumes 1-3, Cold Spring Harbor Laboratory Press; 3rd edition, Jan. 15, 2001, ISBN-10: 0879695773; and Frederick M. Ausubel, Roger Brent, Robert E. Kingston, David D. Moore, J. G. Seidman, Kevin Struhl (Editors), Current Protocols in Molecular Biology, Volumes 1-3, John Wiley & Sons, 1993, ISBN-10: 0471306614; both of which are incorporated herein by reference in their entirety for disclosure of assay methods.

Accordingly, some aspects of the invention relate to the identification of a biomarker, for example, a genomic mutation or a protein biomarker, as a cause of FSGS. In some embodiments, identification of a FSGS biomarker is achieved by comparative genomic or exomic sequencing of consanguineous subjects, some of which are diagnosed with FSGS. Comparison of the genomic or exomic information is used, in some embodiments, to pinpoint a mutation responsible for the disease in the affected subjects. For example, a recessive mutation causing FSGS can be identified by comparing genomic or exomic information from consanguineous subjects, wherein a region that is found to be identical by descent (IBD) in affected subjects, but not in non-affected subjects, is indicated to harbor the recessive mutation causing FSGS. In some embodiments, a mutation causing FSGS is further pinpointed by comparing IBD regions implicated in FSGS across families. Genomic regions in which FSGS-implicated IBD regions from different families overlap are indicated to harbor a recessive mutation causing FSGS. In some embodiments, one or more deletions and/or other mutations involving part or all of one or both alleles of the NPHP1 gene and/or surrounding locus may be detected directly in a patient sample or in a nucleic acid sample that is isolated and/or purified from a patient sample. Any suitable assay (e.g., any assay involving a specific hybridization step) may be used. In some embodiments, one or more nucleic acids (e.g., one or more oligonucleotides of any suitable size, for example, 5-10, 10-20, 20-30, 30-40, 40-50, 50-60, 60-70, 70-80, 80-90, 90-100 nucleotides long, or longer or shorter) may be used. Depending on the assay format, the oligonucleotide(s) may be free in solution, immobilized on a solid support, or in any other format, or any combination thereof).

In some embodiments, aspects of the invention relate to one or more reagents that are useful for assaying a biological sample from a subject (e.g., a human subject) for the presence of one or more indicia of loss of a functional NPHP1 gene or protein. Accordingly, in some embodiments, aspects of the invention relate to one or more oligonucleotides, antibodies, or other reagents that can be used to assay for the presence or structure of an NPHP1 gene, mRNA, protein, function, or a combination thereof. In some embodiments, one or more primers (e.g., sequencing primers, amplification primers, capture primers, etc.) may be complementary to one or the other strand of a nucleic acid in or flanking of the NPHP1 gene (e.g., a nucleic acid having a sequence provided by genomic locus NG_{—}008287 on chromosome 2 that can be found at ACCESSION NG_{—}008287; VERSION NG_{—}008287.1, GI:194440661). In some embodiments, a nucleic acid having a sequence that is complementary to a portion of an NPHP1 mRNA (e.g., one of the ones described herein) may be used. In some embodiments, an antibody may be a monoclonal, polyclonal, recombinant, or other antibody (e.g., single-chained). An antibody may be selective of specific for one or more NPHP1 wild-type or mutant protein epitopes.

It should be appreciated that an assay may involve comparing a nucleic acid and/or protein level to a reference level (e.g., indicative of wild-type or mutant NPHP1, for example of heterozygous, or homozygous wild-type or mutant NPHP1).

Some embodiments of this invention relate to a diagnostic kit. In some embodiments, a kit according to aspects of this invention contains reagents useful for detecting a FSGS biomarker, for example, reagents useful for performing a molecular detection assay, for example, a PCR, RT-PCR, western blot, northern blot, SNP assay, etc.

In some embodiments, a kit for the detection of a FSGS biomarker comprises a PCR primer or primer pair hybridizing to a genomic sequence deleted in FSGS patients. In some embodiments, the kit further comprises a PCR reagent, for example, a PCR buffer, a PCR polymerase, nucleotides, and/or a magnesium salt.

Aspects of the invention relate to a recessive familial form of FSGS in humans that is not associated with any of the known genetic markers of FSGS (e.g., the ones described herein). In some embodiments, aspects of the invention relate to assisting in the diagnosis of FSGS in a subject that has one or more physiological symptoms that may be indicative of FSGS without needing to perform a biopsy. For example, in some embodiments, one or more clinical hallmarks of FSGS may be identified in a subject. However, the clinical hallmarks alone may not be sufficient to diagnose FSGS without a biopsy. In some embodiments, aspects of the invention may be useful to assist in the diagnosis of FSGS based on the clinical hallmarks without requiring a tissue biopsy. Examples of clinical hallmarks include, but are not limited to, one or more of the following: proteinuria; nephrotic syndrome; a progressive loss of renal function; hypertension; abnormal (e.g., higher than normal) serum creatinine, urine protein, and/or urine microalbumin excretion; end-stage renal disease without another cause; elevated urine microalbumin excretion without another cause (microalbumin >20 mg/g creatinine); foamy urine, and/or edema of the legs and/or other parts of the body. In some embodiments, twenty-four-hour urine protein excretion may be estimated from a spot urine protein to creatinine ratio. However, any suitable assay may be used as aspects of the invention are not limited in this respect.

In some embodiments, a biopsy also may be performed. A diagnosis of FSGS based on renal biopsy may include detecting the presence of areas of glomerular sclerosis and tuft collapse that are both focal and segmental. In some embodiments, segmental hyalinosis, glomerular deposits that are positive for immunoglobulin M and/or C3 by immunofluorescence microscopy, and epithelial cell foot process effacement by electron microscopy may be seen but are not required to make the diagnosis.

Accordingly, in some embodiments aspects of the invention may be used to assist in the diagnosis of FSGS in subjects identified as having one or more other risk factors for FSGS. In some embodiments, a subject may have a family history of FSGS. In some embodiments, a subject may have one or more clinical indicia described herein. In some embodiments, a subject may be subjected to a diagnostic method provided herein or monitored for the presence or absence of an FSGS as described herein, before symptoms of renal disease are manifest in the subject. For example, in some embodiments, a subject with a family history of renal disease, for example, with a family history of FSGS is subjected to a diagnostic method for FSGS or risk of developing FSGS as described herein without the subject exhibiting any signs of kidney disease. In some embodiments, a subject from a family affected by FSGS is subjected to a diagnostic method as described herein shortly after birth, or at about 1, about 2, about 3, about 4, about 5, about 6, about 9, about 12, about 18, about 24, about 36, about 48, or about 50 months of age. In some embodiments, a subject is subjected to a diagnostic method as described herein at about 1, about 2, about 3, about 4, about 5, about 6, about 7, about 8, about 9, about 10, about 11, about 12, about 13, about 14, about 15, about 16, about 17, about 18, about 19, bout 20, or about 21 years of age. In some embodiments, an adult subject of 21 years or older is subjected to a diagnostic method as disclosed herein. In some embodiments, a method is provided comprising determining if a subject not exhibiting clinical symptoms of kidney disease, for example, of FSGS, has a loss of function in the NPHP1 locus, a loss of function mutation in the NPHP1 gene, or an elevated level of Alpha 1 antitrypsin, beta-2 glycoprotein, alpha-1 microglobulin, transthyretin precursor (Prealbumin), apolipoprotein E precursor, apolipoprotein A IV precursor, serotransferrin precursor, and/or Vitamin D binding protein precursor, and, if the subject is found to have such a loss of function or elevated level of protein, then a clinical intervention for the delay of the onset of FSGS is administered to the subject, and/or the subject is monitored for signs of kidney disease, for example, for FSGS, at an elevated frequency as compared to a subject not determined to have the loss of function or elevated protein levels.

However, in some embodiments, the detection of one or more NPHP1 abnormalities as described herein may be used to identify a subject as being at risk for FSGS even in the absence of any other clinical indicia or family history for FSGS. In some embodiments, aspects of the invention relate to methods of classifying FSGS patients according to whether they have FSGS associated with an NPHP1 deletion or mutation (e.g., homozygous, heterozygous, etc.). In some embodiments, information about the NPHP1 status of a subject may be useful in the selection of a treatment for or a prevention of FSGS associated symptoms.

In some embodiments, aspects of the invention relate to one or more therapeutic or treatment options or recommendations for a subject identified as having one or more NPHP1 abnormalities (regardless of whether the subject has any other risk factors for FSGS).

It should be appreciated that NPHP1 mutations or deletions may be associated with juvenile nephronophthisis. However, in some embodiments, NPHP1 deletions or other mutations may be associated with FSGS (e.g., familial FSGS) regardless of the age of onset. Accordingly, in some embodiments, aspects of the invention may be used to assist in the diagnosis of FSGS in adults (e.g., subjects older than 16, older than 18, older than 20 years of age, or older), particularly adults with certain symptoms (e.g., any one or more of those described above or herein). However, in some embodiments FSGS (or a risk therefore) may be identified in subjects (e.g., adult subjects) that do not yet have any symptoms (e.g., of reduced renal function).

In some embodiments, aspects of the invention relate to methods of identifying familial FSGS (or a risk thereof) in subjects. In some embodiments, aspects of the inventions may be used to provide genetic counseling (e.g., to homozygous or heterozygous NPHP1 defective individuals).

In some embodiments, aspects of the invention relate to genetic loci on chromosome 2 (e.g., the NPHP1 gene or surrounding loci) that are associated with a novel recessive familial form of FSGS. In some embodiments, one or more markers (e.g., alleles, SNPs, other polymorphisms, ESTs, splice variants, deletions, loss-of-function mutations, or other markers, or any combination thereof) are provided that can be use to identify subjects at risk of developing FSGS and/or of having children at risk of FSGS or of being carriers of FSGS. For example, in some embodiments, this invention provides a loss-of-function mutation or a deletion in the NPHP1 gene as a diagnostic biomarker indicating the affected subject to have or to be at risk of developing FSGS. Accordingly, aspects of the invention may be useful for assisting in the diagnosis of FSGS, genetic and/or reproductive counseling, and/or therapy decisions for subjects at risk of developing FSGS. In some embodiments, one or more markers may be found at the chromosomal loci shown in the examples or listed in the claims.

In some embodiments, aspects of the invention relate to the detection of one or more serum protein markers that may be used to identify subjects that are at risk of developing or that already have FSGS. In some embodiments, one or more of these markers may be identified in other tissue or biological samples (e.g., in the urine). The examples provide non-limiting illustrations of FSGS-specific serum markers. In some embodiments, one or more serum markers may be used to prepare antibodies (e.g., monoclonal, polyclonal, humanized, etc.) that are useful for diagnostic purposes. It should be appreciated that the presence of one or more FSGS-specific markers in a patient sample may be indicative of the presence or risk for FSGS-related symptoms. In some embodiments, therapeutic recommendations may be made to a patient based on the presence of one or more FSGS-specific serum protein markers.

In some embodiments, one or more protein biomarkers are detected in a body fluid, for example, a blood, serum, or urine sample of a subject. In some embodiments, the protein biomarker is a serum protein biomarker. In some embodiments, the protein biomarker is a member of a complement and/or coagulation cascade, a transport protein, or a zinc finger protein. In some embodiments, the one or more protein biomarker is selected from a group of proteins including Alpha 1 antitrypsin, beta-2 glycoprotein, alpha-1 microglobulin, transthyretin precursor Prealbumin, apolipoprotein E precursor, apolipoprotein A IV precursor, serotransferrin precursor, and Vitamin D binding protein precursor. The sequences for the proteins listed above are well known to those of skill in the related art and representative sequences can be retrieved from public databases, for example, the NCBI database (www.ncbi.nlm.nih.gov). Representative alpha 1 antitrypsin sequences include, for example, sequences related to the entry of GeneID: 5265 (SERPINA1 serpin peptidase inhibitor, Glade A (alpha-1 antiproteinase, antitrypsin), member 1 [Homo sapiens]) in the NCBI database. Representative beta-2 glycoprotein sequences include, for example, sequences related to the entry of GeneID: 350 (APOH apolipoprotein H (beta-2-glycoprotein I) [Homo sapiens]) in the NCBI database. Representative alpha-1 microglobulin sequences include, for example, sequences related to the entry of GeneID: 259 (AMBP alpha-1-microglobulin/bikunin precursor [Homo sapiens]) in the NCBI database. Representative transthyretin sequences include, for example, sequences related to the entry of GeneID: 7276 (TTR transthyretin [Homo sapiens]) in the NCBI database. Representative apolipoprotein E precursor sequences include, for example, sequences related to the entry of GeneID: 348 (APOE apolipoprotein E precursor [Homo sapiens]) in the NCBI database. Representative apolipoprotein A-IV sequences include, for example, sequences related to the entry of GeneID: 337 (APOA4 apolipoprotein A-IV [Homo sapiens]) in the NCBI database. Representative serotransferrin sequences include, for example, sequences related to the entry of GeneID: 7018 (TF transferrin [Homo sapiens]) in the NCBI database. Representative vitamin D binding protein sequences include, for example, sequences related to the entry of GeneID: 2638 (GC group-specific component (vitamin D binding protein) [Homo sapiens]) in the NCBI database.

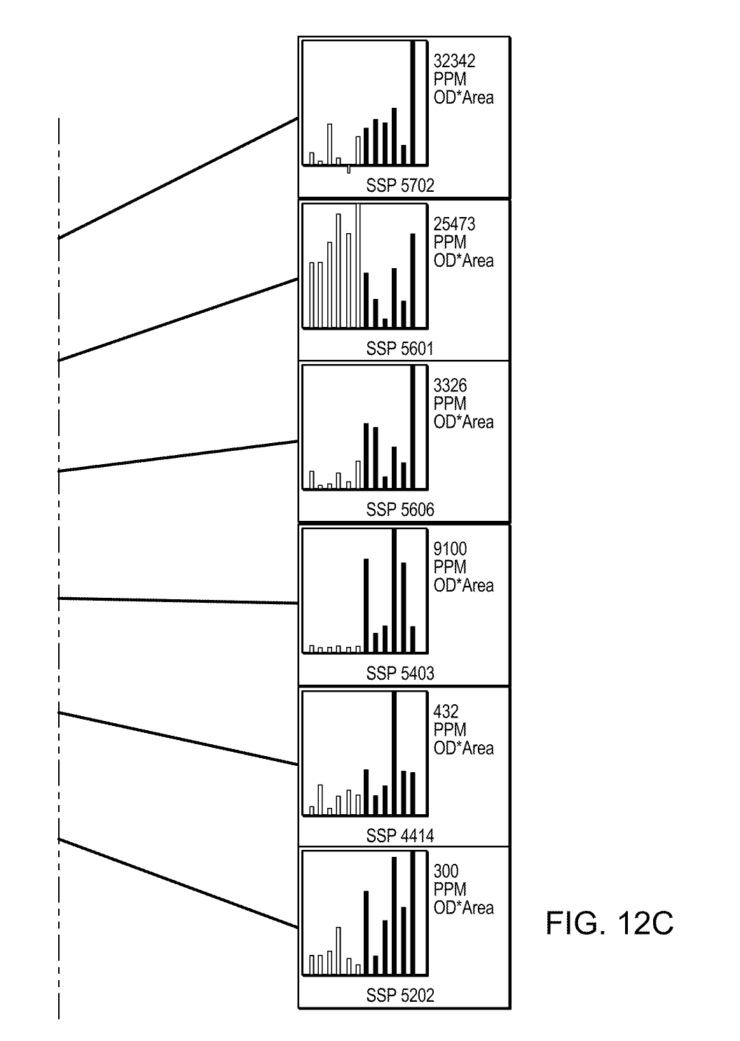

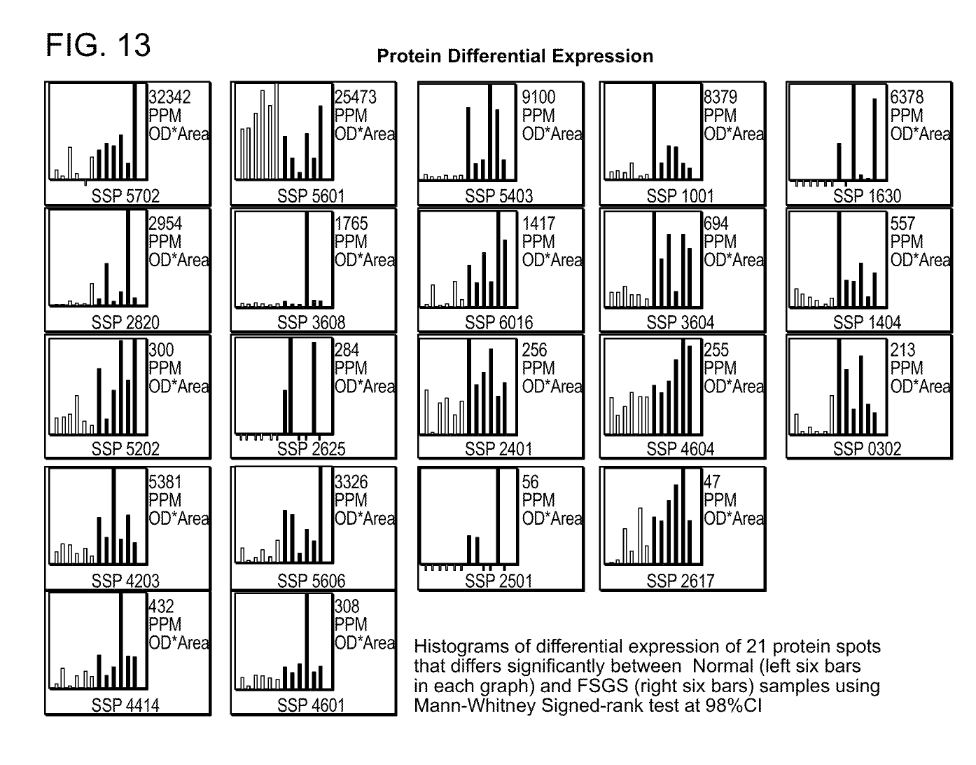

In some embodiments, a biological sample from a subject, for example, a sample comprising a body fluid (e.g., blood serum, urine, saliva, or cerebrospinal fluid), a tissue, or a cell is obtained from and one or more biomarkers, for example, one or more protein biomarkers, one or more nucleic acid biomarkers, or a combination thereof, are assayed. In some embodiments, the level of expression of a protein biomarker or a nucleic acid biomarker is determined and compared to a control or reference level (e.g., a reference level indicative of a subject that does not have FSGS or is not at risk of FSGS). In some embodiments, if the level of expression of the biomarker assayed in the sample from the subject is different (e.g., statistically significantly different) from the control or reference level, then the subject, and/or the subject's progeny, is indicated to be at risk of developing FSGS. In some embodiments, if the level of expression of the biomarker is elevated in the sample from the subject as compared to the reference or control level, then the subject, and/or the subject's progeny, is indicated to be at risk of developing FSGS. In some embodiments, if the level of expression of the biomarker is decreased in the sample from the subject as compared to the reference or control level, then the subject, and/or the subject's progeny, is indicated to be at risk of developing FSGS. In some embodiments, a panel of 2 or more biomarker proteins disclosed herein are assayed in a subject, and an elevated level of at least one of the protein biomarkers in the panel is indicative of FSGS in the subject. In some embodiments, a panel of biomarker proteins disclosed herein are assayed in a subject, and a decreased level of at least one of the protein biomarkers in the panel is indicative of FSGS in the subject. In some embodiments, a panel of biomarker proteins disclosed herein are assayed in a subject, and an elevated level of at least one of these biomarkers and a decreased level of at least one of these biomarkers in the panel is indicative of FSGS in the subject. See, for example, FIG. 13, for an exemplary panel of protein biomarkers. In some embodiments, if the level of expression of the biomarker in the sample from the subject is similar to or the same as the reference or control level, then the subject, and/or the subject's progeny, is not indicated to be at risk of developing FSGS. It should be appreciated that in some embodiments the level of a protein or nucleic acid in a subject sample may be compared to a reference level indicative of FSGS or elevated risk for FSGS in a subject (e.g., a reference level determined in a sample obtained from a subject having or at risk of developing FSGS), as aspects of the invention are not limited in this respect. In this case, a level similar or close to the reference level (e.g., statistically similar to the reference level) may be indicative of FSGS or an elevated risk for FSGS for the subject (or the progeny of the subject).

In some embodiments, a biomarker is a binary value indicating, for example, whether expression of a protein or nucleic acid is detected in the sample from the subject. In some embodiments, a biomarker is a ratio of the expression levels of one or more proteins and/or nucleic acids. In some embodiments, the control or reference level is, or is based on, a level, or an average of levels, of a respective biomarker found in a subject or in subjects not indicated to have, diagnosed with, or suspected to have FSGS or a predisposition for developing FSGS. In some embodiments, the control or reference level is, or is based on, a level of the respective biomarker measured in a control or reference sample assayed in parallel to the sample from the subject. In some embodiments, the control or reference sample contains a known level of the respective biomarker. In some embodiments, the control or reference sample is a sample from a healthy subject. However, in some embodiments a control or reference level is indicative of FSGS or risk for FSGS and/or the control or reference sample is a sample from a subject having FSGS or having one or more risk factors for FSGS (e.g., a deletion or other mutation described herein).

In some embodiments, an elevated level of a protein in a subject as compared to a reference or control level is a level that is higher in the subject, for example, as measured in a biological sample obtained from the subject, to an extent that the difference is statistically significant. Suitable and appropriate methods for the determination of statistical significance in comparing two or more protein levels are well known to those of skill in the art and the invention is not limited in this respect. In some embodiments, a protein level in a subject, or in a biological sample obtained from a subject, is elevated, if it is at least 2 times, at least 3 times, at least 4 times, at least 5 times, at least 6 times, at least 7 times, at least 8 times, at least 9 times, at least 10 times, at least 15 times, at least 20 times, at least 25 times, at least 50 times, or at least 100 times the control or reference level.

In some embodiments, the control or reference level is a level expected or observed in a subject not having FSGS. In some embodiments, the control or reference level is an average level observed in a healthy population of subjects or in a population of subjects not having FSGS. In some embodiments, the control or reference level is an approximate level representing an average level found or expected in a healthy subject.

It should be appreciated that in some embodiments, the genetic and protein markers may be used together to assist in the diagnosis or analysis of a patient. In some embodiments, the genetic and/or protein markers of the invention may be combined with the evaluation of other symptoms and signs of FSGS. For example, in children and some adults, FSGS presents as a nephrotic syndrome, which is characterized by edema (associated with weight gain), hypoalbuminemia (low serum albumin, a protein in the blood), hyperlipidemia and hypertension (high blood pressure). In adults it may also present as kidney failure and proteinuria, without a full-blown nephrotic syndrome. Accordingly, signs and symptoms may be evaluated using one or more of the following assays: urinalysis; blood tests (e.g., cholesterol); and/or kidney biopsy.

Accordingly, in some embodiments, aspects'of the invention relate to evaluating a subject that has one or more symptoms of FSGS (or other symptoms of renal failure) for the presence of an indicia of loss of NPHP1 gene, protein, or function. It should be appreciated that a subject indicated as being at risk for, or predisposed to, FSGS is a subject that has an elevated risk for FSGS relative to an average risk of a subject in a population, or relative to a subject that has no genetic and/or other risk factors for FSGS (e.g., more than 10%, more than 50%, or more than 100% higher than, or about 2 fold, 3 fold, 4 fold, 5 fold, 10 fold, 100 fold, higher than an average risk of a subject in a population, or than a subject that has no genetic and/or other risk factors for FSGS).

In the event that a subject is identified as having or being at risk for FSGS (e.g., based one the identification of one or more genetic or protein markers described herein, alone or in combination with a subject's family history and/or one or more of the subject's clinical symptoms), one or more of the following treatments may be recommended: salt restriction and diuretics (water pills), such as furosemide, for edema; antihypertensives (especially ACEIs)—if the blood pressure is too high; treatment for present hyperlipidemia (e.g. statins, fibrates); aldosterone antagonists to decrease proteinuria and thus offer a degree of reno-protection; corticosteroids, such as prednisone—based on the clinical judgment of physician; or any combination thereof. Cytotoxics, such as cyclophosphamide may be used to induce remission in patients presenting with FSGS refractory to corticosteroids, or in patients who do not tolerate steroids. In some embodiments, a treatment may involve plasmapheresis—blood cleansing using a machine to remove the patient's blood plasma and replacing it with donor plasma.

In some embodiments, a treatment option may include one or more of the following non-limiting examples of treatments or recommendations: avoiding potentially harmful medications that can damage the kidneys, such as non-steroidal anti-inflammatory drugs and Chinese herbal supplements; following a low-sodium diet to help protect the kidneys and lower blood pressure; medications to lower blood pressure and urine protein excretion, such as angiotensin converting enzyme (ACE) inhibitors; and/or steroids or immunosuppressive drugs to help decrease proteinuria and improve kidney function in a minority of patients (however, this may be patient specific since these drugs actually decrease kidney function in some subjects).

In some embodiments, once one or more assays of the invention have been used to assist in the diagnosis of FSGS, a tissue biopsy or other study may assist in the selection of an appropriate therapy. However, it should be appreciated that the etiology of FSGS can have a major influence on therapeutic decision-making. The etiologic hallmark of primary idiopathic FSGS is podocyte injury while secondary types of FSGS (e.g., familial-genetic types, virus-associated FSGS, unilateral renal agenesis, and FSGS associated with renal dysplasia, surgical renal ablation, hypertension, and obesity) develop as a maladaptive response to glomerular “overwork.” The characteristic features of primary idiopathic FSGS include extensive foot process effacement, variable glomerulomegaly, and a clinical presentation consistent with full-blown nephrotic syndrome. In contrast, secondary FSGS is characterized by limited foot process effacement, glomerulomegaly, and proteinuria without hypoalbuminemia. For example, obesity-related glomerulopathy (ORG) is an emerging epidemic (Kidney Int. 2001; 59:1498-1509) and should be distinguished from idiopathic FSGS. Compared with patients with idiopathic FSGS, those with ORG are significantly older, more often Caucasian, and have lower incidences of nephrotic syndrome and nephrotic range proteinuria, higher serum albumin, and lower serum cholesterol levels. On renal biopsy, patients with ORG have more glomerulomegaly, fewer lesions of segmental sclerosis, and less extensive foot process effacement.

In some embodiments, corticosteroids and immunomodulatory agents may be recommended for patients with primary idiopathic FSGS while ACE inhibitors and angiotensin receptor-blockers are more commonly used in patients with secondary FSGS. In some embodiments, improved responsiveness to more prolonged courses of steroids is seen in some subjects. Partial or complete remission may be achieved in about 50% of adults with FSGS treated with long-term corticosteroid therapy (Semin Nephrol. 2000; 20:309-317).

In some embodiments, treatment options may include cyclophosphamide (e.g., for patients with FSGS who do not respond to other immunosuppressive therapies); cyclosporine (e.g., for patients with steroid-resistant or steroid-dependent FSGS); cyclosporine plus low-dose prednisone; tacrolimus; alkylating agents; Mycophenolate mofetil—MMF (e.g., for patients with steroid-resistant FSGS and lowers proteinuria; or any combination of two or more thereof.

In some embodiments, aspects of the invention relate to nucleic acid based therapy (e.g., gene therapy) to provide an NPHP1 function to subjects that are NPHP1 defective in order to reduce the risk of symptoms of FSGS. The nucleic acid therapy may be based on nucleic acid delivery (e.g., DNA, RNA, or other nucleic acid, or any combination thereof), with or without carriers, in the form of a vector, in the form of recombinant cells, in a form that can promote homologous recombination, or in any other suitable form in an amount sufficient to provide a level of NPHP1 that is therapeutically effective. In some embodiments, an NPHP1 protein may be delivered in an amount sufficient to be therapeutically effective. In some embodiments, the nucleic acid and/or protein delivery (e.g., injection) may systemic or targeted to a particular tissue or organ (e.g., kidney or other organ).

In some embodiments, aspects of the invention relate to the importance of considering alternative diagnoses methods based on performing whole-genome genetic evaluation of patients and their families, particularly when meticulous clinical and histopathological information can not be obtained. An approach to diagnose patients with hereditary kidney disease based on recent advances in genetic technologies is provided herein. This approach is useful to effectively evaluate such families with inherited kidney failure, provide accurate diagnosis and shed light on a possibly widely spread allelic variant in consanguineous families with kidney failure presenting with findings consistent with FSGS in Saudi Arabia.

Focal segmental glomerulosclerosis (FSGS) is a histological glomerular phenotype that can be familial, primary (idiopathic), or secondary to a multitude of pathological processes affecting the kidney, including such tubulointerstitial diseases as nephronophthisis. Mutations in a number of distinct nephronophthisis genes (NPHPs) have been described to date.

In some embodiments, two consanguineous unrelated Saudi Arabian families (FAM 001 and FAM012) are described with sequence variants in one of the NPHP genes, namely NPHP1. Affected individuals in the two families presented with end-stage renal disease and clinical and histological features consistent with focal segmental glomerulosclerosis. Since FSGS patients may present atypical radiological findings, making the clinical diagnosis of the genetic syndrome difficult, whole-genome single-nucleotide polymorphism analysis was applied followed by state of the art sequence capture and exome sequencing on genomic DNA samples from these families. This analysis facilitated accurate diagnosis after isolation of homozygosity run of ˜2 Mb. This homozygous run falls between rs6754115 (genomic position 109,328,776) and rs17464100 (genomic position 111,284,252), and is identical in affected subjects from the unrelated families. This approach was adapted for rapid genetic diagnosis of these two consanguineous families.

The NPHP1 gene and NPHP1 gene products, for example NPHP1 mRNAs and NPHP1 proteins are known to those of skill in the art. Representative NPHP1 sequences include, for example, sequences related to the entry of GeneID: 4867 (NPHP1 nephronophthisis 1 (juvenile) [Homo sapiens]) in the NCBI database (www.ncbi.nlm.nih.gov). Some examples of representative NPHP1 sequences are given below:

>gi|189491772|ref|NM_000272.3| Homo sapiens
nephronophthisis 1 (juvenile) (NPHP1),
transcript variant 1, mRNA
CTGGGAGGCGGGCGCACATCGATGGCGTCACCTTCTGGCGCCGCCGGTTG
GTTTCCCTGGCAACTGGAGCAATCAGAGCACCGCAGCCAGGGAGATGCTG
GCGAGACGACAGCGAGATCCTCTCCAGGCCCTGCGGCGCCGCAATCAGGA
GCTGAAGCAACAGGTTGATAGTTTGCTTTCTGAGAGCCAACTGAAAGAAG
CTCTAGAACCCAATAAAAGACAACATATTTATCAAAGATGTATCCAGTTA
AAGCAGGCAATAGATGAAAATAAAAATGCTCTTCAAAAATTAAGCAAAGC
TGATGAATCTGCACCTGTTGCAAACTATAATCAGAGAAAAGAAGAGGAGC
ATACTCTTTTGGACAAGCTTACCCAACAACTGCAGGGCCTTGCTGTGACA
ATAAGCAGAGAAAATATAACTGAAGTTGGGGCACCTACTGAAGAAGAGGA
AGAAAGTGAAAGTGAAGATAGTGAAGACAGTGGTGGGGAGGAAGAAGATG
CAGAGGAGGAAGAGGAAGAGAAAGAGGAAAATGAATCTCACAAATGGTCA
ACCGGTGAAGAATACATCGCTGTTGGAGATTTTACTGCTCAGCAAGTTGG
AGATCTTACATTTAAGAAAGGGGAAATTCTCCTTGTAATTGAAAAAAAAC
CTGATGGTTGGTGGATAGCTAAGGATGCCAAAGGAAATGAAGGTCTTGTT
CCCAGAACCTACCTAGAGCCTTATAGTGAAGAAGAAGAAGGCCAAGAGTC
AAGTGAAGAGGGCAGTGAAGAAGATGTAGAGGCGGTGGATGAAACAGCAG
ATGGAGCAGAAGTTAAGCAAAGAACTGATCCCCACTGGAGTGCTGTTCAG
AAAGCGATTTCAGAGGCGGGCATCTTCTGTCTTGTTAATCATGTCTCGTT
TTGCTACCTAATAGTTCTGATGCGAAATAGGATGGAGACTGTGGAAGACA
CCAATGGATCTGAAACAGGGTTCAGGGCATGGAATGTACAGAGCAGAGGA
CGTATATTTCTGGTTTCTAAGCCTGTGCTCCAACAGATAAACACTGTTGA
TGTGTTAACTACGATGGGAGCTATTCCTGCAGGGTTCAGGCCTTCCACGC
TCTCACAGCTTCTGGAGGAAGGGAATCAATTTCGAGCAAATTACTTCTTA
CAACCAGAGCTCATGCCTTCACAACTGGCCTTCAGAGATCTGATGTGGGA
TGCTACAGAAGGCACTATTAGGTCGAGACCAAGTCGTATTTCATTGATTC
TGACATTATGGAGCTGTAAAATGATTCCTCTTCCAGGAATGAGCATACAG
GTTCTCAGCAGACATGTACGCCTCTGTCTATTTGATGGTAATAAGGTTCT
GAGCAACATTCATACAGTCAGAGCCACATGGCAACCTAAAAAGCCCAAAA
CATGGACCTTTTCTCCCCAGGTTACTCGCATCTTACCATGTTTGCTTGAT
GGTGATTGCTTTATCAGGTCTAATTCTGCATCTCCAGATCTTGGAATATT
ATTTGAACTTGGAATTTCTTATATTCGCAATTCAACTGGTGAAAGAGGAG
AGTTAAGCTGTGGCTGGGTGTTTCTTAAACTTTTTGATGCCAGTGGAGTT
CCTATTCCAGCAAAAACTTATGAGCTTTTCTTGAATGGTGGTACTCCTTA
TGAAAAAGGTATTGAAGTGGACCCTTCAATATCCAGAAGAGCACACGGCA
GTGTTTTCTACCAGATTATGACAATGAGAAGGCAGCCTCAACTTCTAGTG
AAACTGAGATCCTTGAACAGAAGATCAAGAAATGTACTAAGTCTACTGCC
AGAAACATTAATTGGAAATATGTGTTCTATTCACTTGTTGATATTTTATC
GACAAATTCTTGGAGATGTGCTCCTGAAAGACAGGATGAGCTTGCAAAGT
ACTGATTTAATTAGCCATCCCATGCTGGCCACCTTCCCCATGCTCTTGGA
GCAGCCTGATGTGATGGATGCTCTCAGGAGTTCGTGGGCTGGAAAAGAAA
GCACATTAAAAAGATCAGAGAAGAGAGACAAAGAGTTCCTGAAGTCCACG
TTTCTCCTGGTTTACCATGACTGCGTGCTCCCACTTCTCCACTCCACACG
CCTACCCCCATTCAGGTGGGCAGAAGAAGAGACTGAGACTGCACGGTGGA
AAGTTATCACTGACTTCCTTAAGCAAAACCAAGAAAACCAGGGCGCCCTC
CAAGCTCTGCTGTCACCAGACGGAGTTCATGAACCTTTTGACCTTTCAGA
GCAGACCTATGACTTCTTGGGTGAAATGAGAAAGAATGCAGTGTGACAGT
GGCAGCCTCTAGCCCTCAGCTTCCCACGGAATCAGATGGATCCTCCACGA
TTACGTGAATAAAATGATGGAACCAAAAATCACTGTCACTTTACAACTTA
GGTTTTACTCTTTTCTTTCTACAGACCATATTTTTAAAGAAATGTTTATA
CAATAATTTAAATATTTTTTAAAACCATAAAATAAATTTTTATAAGGAAT
ACTGTTATATCTAAATTTAAACAGTATTTATTTTTTCAAAAACAGCTACT
TAAGTTAATGGTATAGATTTCTATAAAAGCAAGATTTTGTCAAAAACTAA
ATTTATGATTATTCAAGAAAGTGAAAAAAACAACCTACAGAATGGGAAAA
CATATTTGCAAATCATCTAACTGATAAAGGTCTAGTATCCAAAATATTTA
AATTTATGAGTGTTAATAAAATTTATCTTGTTCAATGAAGAGGAAGTTAA
AAAAAAAA
>gi|189491773|ref|NM_207181.2| Homo sapiens
nephronophthisis 1 (juvenile) (NPHP1),
transcript variant 2, mRNA
CTGGGAGGCGGGCGCACATCGATGGCGTCACCTTCTGGCGCCGCCGGTTG
GTTTCCCTGGCAACTGGAGCAATCAGAGCACCGCAGCCAGGGAGATGCTG
GCGAGACGACAGCGAGATCCTCTCCAGGCCCTGCGGCGCCGCAATCAGGA
GCTGAAGCAACAGGTTGATAGTTTGCTTTCTGAGAGCCAACTGAAAGAAG
CTCTAGAACCCAATAAAAGACAACATATTTATCAAAGATGTATCCAGTTA
AAGCAGGCAATAGATGAAAATAAAAATGCTCTTCAAAAATTAAGCAAAGC
TGATGAATCTGCACCTGTTGCAAACTATAATCAGAGAAAAGAAGAGGAGC
ATACTCTTTTGGACAAGCTTACCCAACAACTGCAGGGCCTTGCTGTGACA
ATAAGCAGAGAAAATATAACTGAAGTTGGGGCACCTACTGAAGAAGAGGA
AGAAAGTGAAAGTGAAGATAGTGAAGACAGTGGTGGGGAGGAAGAAGATG
CAGAGGAGGAAGAGGAAGAGAAAGAGGAAAATGAATCTCACAAATGGTCA
ACCGGTGAAGAATACATCGCTGTTGGAGATTTTACTGCTCAGCAAGTTGG
AGATCTTACATTTAAGAAAGGGGAAATTCTCCTTGTAATTGAAAAAAAAC
CTGATGGTTGGTGGATAGCTAAGGATGCCAAAGGAAATGAAGGTCTTGTT
CCCAGAACCTACCTAGAGCCTTATAGTGAAGAAGAAGAAGGCCAAGAGTC
AAGTGAAGAGGGCAGTGAAGAAGATGTAGAGGCGGTGGATGAAACAGCAG
ATGGAGCAGAAGTTAAGCAAAGAACTGATCCCCACTGGAGTGCTGTTCAG
AAAGCGATTTCAGAGGCGGGCATCTTCTGTCTTGTTAATCATGTCTCGTT
TTGCTACCTAATAGTTCTGATGCGAAATAGGATGGAGACTGTGGAAGACA
CCAATGGATCTGAAACAGGGTTCAGGGCATGGAATGTACAGAGCAGAGGA
CGTATATTTCTGGTTTCTAAGCCTGTGCTCCAAATAAACACTGTTGATGT
GTTAACTACGATGGGAGCTATTCCTGCAGGGTTCAGGCCTTCCACGCTCT
CACAGCTTCTGGAGGAAGGGAATCAATTTCGAGCAAATTACTTCTTACAA
CCAGAGCTCATGCCTTCACAACTGGCCTTCAGAGATCTGATGTGGGATGC
TACAGAAGGCACTATTAGGTCGAGACCAAGTCGTATTTCATTGATTCTGA
CATTATGGAGCTGTAAAATGATTCCTCTTCCAGGAATGAGCATACAGGTT
CTCAGCAGACATGTACGCCTCTGTCTATTTGATGGTAATAAGGTTCTGAG
CAACATTCATACAGTCAGAGCCACATGGCAACCTAAAAAGCCCAAAACAT
GGACCTTTTCTCCCCAGGTTACTCGCATCTTACCATGTTTGCTTGATGGT
GATTGCTTTATCAGGTCTAATTCTGCATCTCCAGATCTTGGAATATTATT
TGAACTTGGAATTTCTTATATTCGCAATTCAACTGGTGAAAGAGGAGAGT
TAAGCTGTGGCTGGGTGTTTCTTAAACTTTTTGATGCCAGTGGAGTTCCT
ATTCCAGCAAAAACTTATGAGCTTTTCTTGAATGGTGGTACTCCTTATGA
AAAAGGTATTGAAGTGGACCCTTCAATATCCAGAAGAGCACACGGCAGTG
TTTTCTACCAGATTATGACAATGAGAAGGCAGCCTCAACTTCTAGTGAAA
CTGAGATCCTTGAACAGAAGATCAAGAAATGTACTAAGTCTACTGCCAGA
AACATTAATTGGAAATATGTGTTCTATTCACTTGTTGATATTTTATCGAC
AAATTCTTGGAGATGTGCTCCTGAAAGACAGGATGAGCTTGCAAAGTACT
GATTTAATTAGCCATCCCATGCTGGCCACCTTCCCCATGCTCTTGGAGCA
GCCTGATGTGATGGATGCTCTCAGGAGTTCGTGGGCTGGAAAAGAAAGCA
CATTAAAAAGATCAGAGAAGAGAGACAAAGAGTTCCTGAAGTCCACGTTT
CTCCTGGTTTACCATGACTGCGTGCTCCCACTTCTCCACTCCACACGCCT
ACCCCCATTCAGGTGGGCAGAAGAAGAGACTGAGACTGCACGGTGGAAAG
TTATCACTGACTTCCTTAAGCAAAACCAAGAAAACCAGGGCGCCCTCCAA
GCTCTGCTGTCACCAGACGGAGTTCATGAACCTTTTGACCTTTCAGAGCA
GACCTATGACTTCTTGGGTGAAATGAGAAAGAATGCAGTGTGACAGTGGC
AGCCTCTAGCCCTCAGCTTCCCACGGAATCAGATGGATCCTCCACGATTA
CGTGAATAAAATGATGGAACCAAAAATCACTGTCACTTTACAACTTAGGT
TTTACTCTTTTCTTTCTACAGACCATATTTTTAAAGAAATGTTTATACAA
TAATTTAAATATTTTTTAAAACCATAAAATAAATTTTTATAAGGAATACT
GTTATATCTAAATTTAAACAGTATTTATTTTTTCAAAAACAGCTACTTAA
GTTAATGGTATAGATTTCTATAAAAGCAAGATTTTGTCAAAAACTAAATT
TATGATTATTCAAGAAAGTGAAAAAAACAACCTACAGAATGGGAAAACAT
ATTTGCAAATCATCTAACTGATAAAGGTCTAGTATCCAAAATATTTAAAT
TTATGAGTGTTAATAAAATTTATCTTGTTCAATGAAGAGGAAGTTAAAAA
AAAAA
>gi|189491775|ref|NM_001128178.1| Homo sapiens
nephronophthisis 1 (juvenile) (NPHP1),
transcript variant 3, mRNA
CTGGGAGGCGGGCGCACATCGATGGCGTCACCTTCTGGCGCCGCCGGTTG
GTTTCCCTGGCAACTGGAGCAATCAGAGCACCGCAGCCAGGGAGATGCTG
GCGAGACGACAGCGAGATCCTCTCCAGGCCCTGCGGCGCCGCAATCAGGA
GCTGAAGCAACAGGTTGATAGTTTGCTTTCTGAGAGCCAACTGAAAGAAG
CTCTAGAACCCAATAAAAGACAACATATTTATCAAAGATGTATCCAGTTA
AAGCAGGCAATAGATGAAAATAAAAATGCTCTTCAAAAATTAAGCAAAGC
TGATGAATCTGCACCTGTTGCAAACTATAATCAGAGAAAAGAAGAGGAGC
ATACTCTTTTGGACAAGCTTACCCAACAACTGCAGGGCCTTGCTGTGACA
ATAAGCAGAGAAAATATAACTGAAGTTGGGGCACCTACTGAAGAAGAGGA
AGAAAGTGAAAGTGAAGATAGTGAAGACAGTGGTGGGGAGGAAGAAGATG
CAGAGGAGGAAGAGGAAGAGAAAGAGGAAAATGAATCTCACAAATGGTCA
ACCGGTGAAGAATACATCGCTGTTGGAGATTTTACTGCTCAGCAAGTTGG
AGATCTTACATTTAAGAAAGGGGAAATTCTCCTTGTAATTGAAAAAAAAC
CTGATGGTTGGTGGATAGCTAAGGATGCCAAAGGAAATGAAGGTCTTGTT
CCCAGAACCTACCTAGAGCCTTATAGTGAAGAAGAAGAAGGCCAAGAGTC
AAGTGAAGAGGGCAGTGAAGAAGATGTAGAGGCGGTGGATGAAACAGCAG
ATGGAGCAGAAGTTAAGCAAAGAACTGATCCCCACTGGAGTGCTGTTCAG
AAAGCGATTTCAGAGCAGATAAACACTGTTGATGTGTTAACTACGATGGG
AGCTATTCCTGCAGGGTTCAGGCCTTCCACGCTCTCACAGCTTCTGGAGG
AAGGGAATCAATTTCGAGCAAATTACTTCTTACAACCAGAGCTCATGCCT
TCACAACTGGCCTTCAGAGATCTGATGTGGGATGCTACAGAAGGCACTAT
TAGGTCGAGACCAAGTCGTATTTCATTGATTCTGACATTATGGAGCTGTA
AAATGATTCCTCTTCCAGGAATGAGCATACAGGTTCTCAGCAGACATGTA
CGCCTCTGTCTATTTGATGGTAATAAGGTTCTGAGCAACATTCATACAGT
CAGAGCCACATGGCAACCTAAAAAGCCCAAAACATGGACCTTTTCTCCCC
AGGTTACTCGCATCTTACCATGTTTGCTTGATGGTGATTGCTTTATCAGG
TCTAATTCTGCATCTCCAGATCTTGGAATATTATTTGAACTTGGAATTTC
TTATATTCGCAATTCAACTGGTGAAAGAGGAGAGTTAAGCTGTGGCTGGG
TGTTTCTTAAACTTTTTGATGCCAGTGGAGTTCCTATTCCAGCAAAAACT
TATGAGCTTTTCTTGAATGGTGGTACTCCTTATGAAAAAGGTATTGAAGT
GGACCCTTCAATATCCAGAAGAGCACACGGCAGTGTTTTCTACCAGATTA
TGACAATGAGAAGGCAGCCTCAACTTCTAGTGAAACTGAGATCCTTGAAC
AGAAGATCAAGAAATGTACTAAGTCTACTGCCAGAAACATTAATTGGAAA
TATGTGTTCTATTCACTTGTTGATATTTTATCGACAAATTCTTGGAGATG
TGCTCCTGAAAGACAGGATGAGCTTGCAAAGTACTGATTTAATTAGCCAT
CCCATGCTGGCCACCTTCCCCATGCTCTTGGAGCAGCCTGATGTGATGGA
TGCTCTCAGGAGTTCGTGGGCTGGAAAAGAAAGCACATTAAAAAGATCAG
AGAAGAGAGACAAAGAGTTCCTGAAGTCCACGTTTCTCCTGGTTTACCAT
GACTGCGTGCTCCCACTTCTCCACTCCACACGCCTACCCCCATTCAGGTG
GGCAGAAGAAGAGACTGAGACTGCACGGTGGAAAGTTATCACTGACTTCC
TTAAGCAAAACCAAGAAAACCAGGGCGCCCTCCAAGCTCTGCTGTCACCA
GACGGAGTTCATGAACCTTTTGACCTTTCAGAGCAGACCTATGACTTCTT
GGGTGAAATGAGAAAGAATGCAGTGTGACAGTGGCAGCCTCTAGCCCTCA
GCTTCCCACGGAATCAGATGGATCCTCCACGATTACGTGAATAAAATGAT
GGAACCAAAAATCACTGTCACTTTACAACTTAGGTTTTACTCTTTTCTTT
CTACAGACCATATTTTTAAAGAAATGTTTATACAATAATTTAAATATTTT
TTAAAACCATAAAATAAATTTTTATAAGGAATACTGTTATATCTAAATTT
AAACAGTATTTATTTTTTCAAAAACAGCTACTTAAGTTAATGGTATAGAT
TTCTATAAAAGCAAGATTTTGTCAAAAACTAAATTTATGATTATTCAAGA
AAGTGAAAAAAACAACCTACAGAATGGGAAAACATATTTGCAAATCATCT
AACTGATAAAGGTCTAGTATCCAAAATATTTAAATTTATGAGTGTTAATA
AAATTTATCTTGTTCAATGAAGAGGAAGTTAAAAAAAAAA
>gi|189491777|ref|NM_001128179.1| Homo sapiens
nephronophthisis 1 (juvenile) (NPHP1),
transcript variant 4, mRNA
CTGGGAGGCGGGCGCACATCGATGGCGTCACCTTCTGGCGCCGCCGGTTG
GTTTCCCTGGCAACTGGAGCAATCAGAGCACCGCAGCCAGGGAGATGCTG
GCGAGACGACAGCGAGATCCTCTCCAGGCCCTGCGGCGCCGCAATCAGGA
GCTGAAGCAACAGGTTGATAGTTTGCTTTCTGAGAGCCAACTGAAAGAAG
CTCTAGAACCCAATAAAAGACAACATATTTATCAAAGAGTTGGGGCACCT
ACTGAAGAAGAGGAAGAAAGTGAAAGTGAAGATAGTGAAGACAGTGGTGG
GGAGGAAGAAGATGCAGAGGAGGAAGAGGAAGAGAAAGAGGAAAATGAAT
CTCACAAATGGTCAACCGGTGAAGAATACATCGCTGTTGGAGATTTTACT
GCTCAGCAAGTTGGAGATCTTACATTTAAGAAAGGGGAAATTCTCCTTGT
AATTGAAAAAAAACCTGATGGTTGGTGGATAGCTAAGGATGCCAAAGGAA
ATGAAGGTCTTGTTCCCAGAACCTACCTAGAGCCTTATAGTGAAGAAGAA
GAAGGCCAAGAGTCAAGTGAAGAGGGCAGTGAAGAAGATGTAGAGGCGGT
GGATGAAACAGCAGATGGAGCAGAAGTTAAGCAAAGAACTGATCCCCACT
GGAGTGCTGTTCAGAAAGCGATTTCAGAGATAAACACTGTTGATGTGTTA
ACTACGATGGGAGCTATTCCTGCAGGGTTCAGGCCTTCCACGCTCTCACA
GCTTCTGGAGGAAGGGAATCAATTTCGAGCAAATTACTTCTTACAACCAG
AGCTCATGCCTTCACAACTGGCCTTCAGAGATCTGATGTGGGATGCTACA
GAAGGCACTATTAGGTCGAGACCAAGTCGTATTTCATTGATTCTGACATT
ATGGAGCTGTAAAATGATTCCTCTTCCAGGAATGAGCATACAGGTTCTCA
GCAGACATGTACGCCTCTGTCTATTTGATGGTAATAAGGTTCTGAGCAAC
ATTCATACAGTCAGAGCCACATGGCAACCTAAAAAGCCCAAAACATGGAC
CTTTTCTCCCCAGGTTACTCGCATCTTACCATGTTTGCTTGATGGTGATT
GCTTTATCAGGTCTAATTCTGCATCTCCAGATCTTGGAATATTATTTGAA
CTTGGAATTTCTTATATTCGCAATTCAACTGGTGAAAGAGGAGAGTTAAG
CTGTGGCTGGGTGTTTCTTAAACTTTTTGATGCCAGTGGAGTTCCTATTC
CAGCAAAAACTTATGAGCTTTTCTTGAATGGTGGTACTCCTTATGAAAAA
GGTATTGAAGTGGACCCTTCAATATCCAGAAGAGCACACGGCAGTGTTTT
CTACCAGATTATGACAATGAGAAGGCAGCCTCAACTTCTAGTGAAACTGA
GATCCTTGAACAGAAGATCAAGAAATGTACTAAGTCTACTGCCAGAAACA
TTAATTGGAAATATGTGTTCTATTCACTTGTTGATATTTTATCGACAAAT
TCTTGGAGATGTGCTCCTGAAAGACAGGATGAGCTTGCAAAGTACTGATT
TAATTAGCCATCCCATGCTGGCCACCTTCCCCATGCTCTTGGAGCAGCCT
GATGTGATGGATGCTCTCAGGAGTTCGTGGGCTGGAAAAGAAAGCACATT
AAAAAGATCAGAGAAGAGAGACAAAGAGTTCCTGAAGTCCACGTTTCTCC
TGGTTTACCATGACTGCGTGCTCCCACTTCTCCACTCCACACGCCTACCC
CCATTCAGGTGGGCAGAAGAAGAGACTGAGACTGCACGGTGGAAAGTTAT
CACTGACTTCCTTAAGCAAAACCAAGAAAACCAGGGCGCCCTCCAAGCTC
TGCTGTCACCAGACGGAGTTCATGAACCTTTTGACCTTTCAGAGCAGACC
TATGACTTCTTGGGTGAAATGAGAAAGAATGCAGTGTGACAGTGGCAGCC
TCTAGCCCTCAGCTTCCCACGGAATCAGATGGATCCTCCACGATTACGTG
AATAAAATGATGGAACCAAAAATCACTGTCACTTTACAACTTAGGTTTTA
CTCTTTTCTTTCTACAGACCATATTTTTAAAGAAATGTTTATACAATAAT
TTAAATATTTTTTAAAACCATAAAATAAATTTTTATAAGGAATACTGTTA
TATCTAAATTTAAACAGTATTTATTTTTTCAAAAACAGCTACTTAAGTTA
ATGGTATAGATTTCTATAAAAGCAAGATTTTGTCAAAAACTAAATTTATG
ATTATTCAAGAAAGTGAAAAAAACAACCTACAGAATGGGAAAACATATTT
GCAAATCATCTAACTGATAAAGGTCTAGTATCCAAAATATTTAAATTTAT
GAGTGTTAATAAAATTTATCTTGTTCAATGAAGAGGAAGTTAAAAAAAAA
A
>gi|46397398|ref|NP_000263.2|
nephrocystin-1 isoform 1 [Homo sapiens]
MLARRQRDPLQALRRRNQELKQQVDSLLSESQLKEALEPNKRQHIYQRCI
QLKQAIDENKNALQKLSKADESAPVANYNQRKEEEHTLLDKLTQQLQGLA
VTISRENITEVGAPTEEEEESESEDSEDSGGEEEDAEEEEEEKEENESHK
WSTGEEYIAVGDFTAQQVGDLTFKKGEILLVIEKKPDGWWIAKDAKGNEG
LVPRTYLEPYSEEEEGQESSEEGSEEDVEAVDETADGAEVKQRTDPHWSA
VQKAISEAGIFCLVNHVSFCYLIVLMRNRMETVEDTNGSETGFRAWNVQS
RGRIFLVSKPVLQQINTVDVLTTMGAIPAGFRPSTLSQLLEEGNQFRANY
FLQPELMPSQLAFRDLMWDATEGTIRSRPSRISLILTLWSCKMIPLPGMS
IQVLSRHVRLCLFDGNKVLSNIHTVRATWQPKKPKTWTFSPQVTRILPCL
LDGDCFIRSNSASPDLGILFELGISYIRNSTGERGELSCGWVFLKLFDAS
GVPIPAKTYELFLNGGTPYEKGIEVDPSISRRAHGSVFYQIMTMRRQPQL
LVKLRSLNRRSRNVLSLLPETLIGNMCSIHLLIFYRQILGDVLLKDRMSL
QSTDLISHPMLATFPMLLEQPDVMDALRSSWAGKESTLKRSEKRDKEFLK
STFLLVYHDCVLPLLHSTRLPPFRWAEEETETARWKVITDFLKQNQENQG
ALQALLSPDGVHEPFDLSEQTYDFLGEMRKNAV
>gi|189491774|ref|NP_997064.2|
nephrocystin-1 isoform 2 [Homo sapiens]
MLARRQRDPLQALRRRNQELKQQVDSLLSESQLKEALEPNKRQHIYQRCI
QLKQAIDENKNALQKLSKADESAPVANYNQRKEEEHTLLDKLTQQLQGLA
VTISRENITEVGAPTEEEEESESEDSEDSGGEEEDAEEEEEEKEENESHK
WSTGEEYIAVGDFTAQQVGDLTFKKGEILLVIEKKPDGWWIAKDAKGNEG
LVPRTYLEPYSEEEEGQESSEEGSEEDVEAVDETADGAEVKQRTDPHWSA
VQKAISEAGIFCLVNHVSFCYLIVLMRNRMETVEDTNGSETGFRAWNVQS
RGRIFLVSKPVLQINTVDVLTTMGAIPAGFRPSTLSQLLEEGNQFRANYF
LQPELMPSQLAFRDLMWDATEGTIRSRPSRISLILTLWSCKMIPLPGMSI
QVLSRHVRLCLFDGNKVLSNIHTVRATWQPKKPKTWTFSPQVTRILPCLL
DGDCFIRSNSASPDLGILFELGISYIRNSTGERGELSCGWVFLKLFDASG
VPIPAKTYELFLNGGTPYEKGIEVDPSISRRAHGSVFYQIMTMRRQPQLL
VKLRSLNRRSRNVLSLLPETLIGNMCSIHLLIFYRQILGDVLLKDRMSLQ
STDLISHPMLATFPMLLEQPDVMDALRSSWAGKESTLKRSEKRDKEFLKS
TFLLVYHDCVLPLLHSTRLPPFRWAEEETETARWKVITDFLKQNQENQGA
LQALLSPDGVHEPFDLSEQTYDFLGEMRKNAV
>gi|189491776|ref|NP_001121650.1|
nephrocystin-1 isoform 3 [Homo sapiens]
MLARRQRDPLQALRRRNQELKQQVDSLLSESQLKEALEPNKRQHIYQRCI
QLKQAIDENKNALQKLSKADESAPVANYNQRKEEEHTLLDKLTQQLQGLA
VTISRENITEVGAPTEEEEESESEDSEDSGGEEEDAEEEEEEKEENESHK
WSTGEEYIAVGDFTAQQVGDLTFKKGEILLVIEKKPDGWWIAKDAKGNEG
LVPRTYLEPYSEEEEGQESSEEGSEEDVEAVDETADGAEVKQRTDPHWSA
VQKAISEQINTVDVLTTMGAIPAGFRPSTLSQLLEEGNQFRANYFLQPEL
MPSQLAFRDLMWDATEGTIRSRPSRISLILTLWSCKMIPLPGMSIQVLSR
HVRLCLFDGNKVLSNIHTVRATWQPKKPKTWTFSPQVTRILPCLLDGDCF
IRSNSASPDLGILFELGISYIRNSTGERGELSCGWVFLKLFDASGVPIPA
KTYELFLNGGTPYEKGIEVDPSISRRAHGSVFYQIMTMRRQPQLLVKLRS
LNRRSRNVLSLLPETLIGNMCSIHLLIFYRQILGDVLLKDRMSLQSTDLI
SHPMLATFPMLLEQPDVMDALRSSWAGKESTLKRSEKRDKEFLKSTFLLV
YHDCVLPLLHSTRLPPFRWAEEETETARWKVITDFLKQNQENQGALQALL
SPDGVHEPFDLSEQTYDFLGEMRKNAV
>gi|l89491778|ref|NP_001121651.1|
nephrocystin-1 isoform 4 [Homo sapiens]
MLARRQRDPLQALRRRNQELKQQVDSLLSESQLKEALEPNKRQHIYQRVG
APTEEEEESESEDSEDSGGEEEDAEEEEEEKEENESHKWSTGEEYIAVGD
FTAQQVGDLTFKKGEILLVIEKKPDGWWIAKDAKGNEGLVPRTYLEPYSE
EEEGQESSEEGSEEDVEAVDETADGAEVKQRTDPHWSAVQKAISEINTVD
VLTTMGAIPAGFRPSTLSQLLEEGNQFRANYFLQPELMPSQLAFRDLMWD
ATEGTIRSRPSRISLILTLWSCKMIPLPGMSIQVLSRHVRLCLFDGNKVL
SNIHTVRATWQPKKPKTWTFSPQVTRILPCLLDGDCFIRSNSASPDLGIL
FELGISYIRNSTGERGELSCGWVFLKLFDASGVPIPAKTYELFLNGGTPY
EKGIEVDPSISRRAHGSVFYQIMTMRRQPQLLVKLRSLNRRSRNVLSLLP
ETLIGNMCSIHLLIFYRQILGDVLLKDRMSLQSTDLISHPMLATFPMLLE
QPDVMDALRSSWAGKESTLKRSEKRDKEFLKSTFLLVYHDCVLPLLHSTR
LPPFRWAEEETETARWKVITDFLKQNQENQGALQALLSPDGVHEPFDLSE
QTYDFLGEMRKNAV

It will be appreciated by the skilled artisan that any of the nucleotide or amino acid sequences provided may be used to design reagents of the invention. For example, those of skill in the art will be able to generate primers for PCR-based detection of the provided nucleic acid sequences using methods well known and routinely used in the art.

It was evident from the genotyping experiment that none of the known loci for FSGS were within segments of homozygosity in the first family. Genotyping on the second family clearly indicated the involvement of a homozygous run encompassing a small number of genes, one of which is NPHP1. NPHP1 locus deletion causes a familial form a of nephronophthisis. Exome capture and sequencing was performed on affected subject RKH-5 from FAM001. Given the knowledge extracted from the genotyping data, a homozygous disease-causing mutation was anticipated within the ˜2 Mb homozygous run that is shared between the two unrelated consanguineous families. This made identifying the deletion easier since the wealth of data from whole exome sequencing experiments can be overwhelming; for example, it was previously reported that ˜8,000 is the average number for novel homozygous SNPs per exome analysis. In the analysis a total of 36,792,066 74 bp-reads were generated and 99.4% aligned to the human reference sequence and the targeted bases constituted ˜41% of all bases read, which is consistent with previous reports. The exome data in this region for RKH-5 also was compared to that of another subject (KFH-41). The mean exome sequencing coverage for both RKH-5 and KFH-41 was 32×, while in the deletion loci there was zero coverage in RKH-5 and a mean exomic coverage of 75× in KFH-41.

Chronic kidney injuries, such as nephronophthisis, may result in secondary glomerulosclerosis. This is identified by variable histological staining methods under light microscopy as segmental or global scarring of the glomeruli. When significant chronic kidney disease has developed, as in the case of the affected subjects in FAM001 and FAM012, determining the underlying cause of glomerulosclerosis from the biopsy findings may be a difficult task. The histological finding of FSGS may be misinterpreted as part of glomerulosclerosis caused by a primary injury. This may have occurred in at least one of the cases described in this report (KFH-46). Examination of kidney biopsy by electron microscopy can assist to distinguish between phenotypes resulting from podocyte injury, such as primary and familial FSGS, and other phenotypes where podocyte injury is a result of other damaged parts of the kidney (secondary glomerulosclerosis). In patients with primary FSGS or minimal change disease, electron microscopy examination typically shows diffuse podocyte foot-process effacement, along with other signs of podocyte injury, including cytoplasmic vacuolization and microvillous change. These patterns were consistent with the findings seen in affected subject KFH-46.

Adding to the complexity of the diagnosis is that when glomerulosclerosis is secondary to other forms of kidney damage, signs of podocyte injury may be focal and segmental and most likely to be seen in glomeruli undergoing senescence. However, the degree of foot-process effacement can be variable and thus cannot be used reliably to distinguish primary from secondary FSGS. Moreover, In some hereditary glomerular diseases, particularly the autosomal dominant and later-onset forms of familial FSGS, such as that caused by ACTN4 mutations, the podocyte injury may be less severe and often shows a focal and segmental distribution, thus making the distinction from secondary FSGS difficult (15).

Nine nephronophthisis genes (NPHPs) have been described to date and mutations in 6 NPHP genes have been described as nephronophthisis-causing mutations. NPHP1 is responsible for the vast majority (˜85%) of the purely renal form of nephronophthisis. NPHP1 encodes a protein (nephrocystin), which interacts and form a complex with at least 3 other NPHP proteins, nephrocystin 3 (NPHP3), nephrocystin 4 (NPHP4) and inversin (INVS). It also interacts with other proteins involved in cell-cell and cell-matrix signaling such as filamin, tensin, and tubulin, suggesting that nephrocystins have a role in the integrity and architecture of renal tubular epithelial cells (16). The most frequent mutation observed in familial juvenile nephronophthisis is a large homozygous deletion encompassing the whole NPHP1 gene on chromosome 2q13. The NPHP1 deletion was characterized by Saunier et al., 2000 (17) and was concluded in that study to be a result of homologous recombination between direct repeats; NPHP1 gene is flanked by two large inverted repeats of ˜330 kb, the distal one interrupted by a ˜45 kb sequence, which is in turn directly repeated upstream of the NPHP1 gene. The deletion breakpoints were localized within the 45 kb repeats which resulted from unequal recombination event between two homologous-nonallelic copies of 45 kb repeats (17). The unequal recombination between two homologous but nonallelic 45 kb repeats occurs either by chromosome misalignment, followed by an unequal crossing over, or by the formation of a loop structure on a single chromosome resulting breakpoints that remove the NPHP1 locus.

Previous haplotype analyses by Konrad et al., 1996 (18) in NPHP1 families strongly suggested that the deletions detected in those families were not due to a founder effect. In other words the findings by Konrad et al. suggested the NPHP1 deletions in their study was a recurrent event and was inherited from multiple ancestors. The findings in the genotyping experiment, however, indicate that the deletion was an event that occurred in a common ancestor and the haplotype was passed through many generations to the two unrelated Saudi Arabian families. The Affymetrix 250K SNP array contained 36 SNPs from within the homozygous run of ˜2 Mb (between rs6754115 at genomic position 109,328,776 and rs17464100 at genomic position 111,284,252) which were found homozygous in affected subjects from the two unrelated families. This suggests the possibility that this deletion is widely spread in the population where these two families come from and imply its involvement in the development of CRF in Saudi Arabia.

Quantitative proteinuria analysis and early kidney biopsy pathology are key diagnostic procedures for the detection of chronic kidney failure at early stages. These analyses are provided mainly in central hospitals in major cities of Saudi Arabia and delay in the diagnosis amongst large number of patients is observed. This is because of the individual's latency of recognizing symptoms combined with the need to refer patients from rural health facilities to central specialized hospitals (20). In addition, obtaining kidney biopsy is a relatively intrusive procedure that may seem problematic and trigger avoidance-attitude in individuals form rural communities; asymptomatic individuals from high risk groups, such as members of consanguineous families with history of CRF, are even at farther bay from early diagnosis. Establishment of diagnosis based on genetic analysis on DNA samples from peripheral blood has the advantage of providing ease and accuracy in addition to being less intrusive. The utilization of high throughput analyses such as that presented in this report is providing clues of the genetic causes of CRF despite the inadequacies seen in the obtained clinical and pathological data. More importantly this approach facilitated the design of an efficient genetic evaluation assay that will be useful for screening individuals with heritable kidney diseases in Saudi Arabia, and pave the road for establishing a program for genetic counseling.

This provides evidence that this deletion is widely spread in the families' geographical regions, and implies its significant involvement in the development of chronic kidney failure in Saudi Arabia. It will be appreciated by those of skill in the art that the invention is not limited to subjects from the geographical region of Saudi Arabia. In some embodiments, aspects of this invention relate to detection or diagnosis of FSGS in subjects from any region of the world. In some embodiments, a subject is selected to be subjected to a diagnostic method or assay as provided herein based on the subject having a family history of FSGS, e.g., having one or more family member affected by FSGS. Aspects of the invention emphasize the importance performing genetic screening for this NPHP allele in CRF patients and outline an assay for this purpose.

In some embodiments, aspects of the invention relate to animal models (e.g., having one or more deletions or other loss of function mutations in one or both alleles of the NPHP1 gene) for studying and/or evaluating the disease and/or candidate drugs.

Some aspects of this invention relate to protein biomarkers that are useful in the diagnosis and the treatment of FSGS. Some aspects of this invention are based on the surprising discovery that certain serum protein levels are dysregulated in individuals with FSGS and that a dysregulation of such serum protein levels is indicative of an individual having FSGS. In some embodiments, dysregulation is an increased level of the protein as compared to a level found or expected in an individual not affected by FSGS. In some embodiments, dysregulation is a decreased level of the protein as compared to a level found or expected in an individual not affected with FSGS. Some embodiments provide useful biomarkers for FSGS diagnostics. Some embodiments provide methods for the diagnosis of FSGS in a subject based on a detection of an elevated or decreased protein level in the subject as compared to a level found or expected in a subject not affected with FSGS.

In some embodiments, the protein biomarker useful for the diagnosis of FSGS is alpha 1 antitrypsin, beta-2 glycoprotein, alpha-1 microglobulin, transthyretin, or a precursor thereof, apolipoprotein E, or a precursor thereof, apolipoprotein A IV, or a precursor thereof, serotransferrin, or a precursor thereof, or Vitamin D binding protein, or a precursor thereof. In some embodiments, the protein biomarker for the diagnosis of FSGS is an overexpression or an increased serum level of any of the proteins listed above, of all of the proteins listed above, or of any combination of the proteins listed above.

In some embodiments, a method for diagnosing FSGS is provided, comprising obtaining a biological sample from a subject; determining a level of alpha 1 antitrypsin, beta-2 glycoprotein, alpha-1 microglobulin, transthyretin, or a precursor thereof, apolipoprotein E, or a precursor thereof, apolipoprotein A IV, or a precursor thereof, serotransferrin, or a precursor thereof, and/or Vitamin D binding protein, in the biological sample; and comparing the level determined in the biological sample to a control level, for example, a level found or expected in a subject not affected with FSGS. In some embodiments, if the level of alpha 1 antitrypsin, beta-2 glycoprotein, alpha-1 microglobulin, transthyretin, or a precursor thereof, apolipoprotein E, or a precursor thereof, apolipoprotein A IV, or a precursor thereof, serotransferrin, or a precursor thereof, and/or Vitamin D binding protein determined in the biological sample obtained from the subject is higher than the control level, then the subject is indicated to have FSGS. In some embodiments, the biological sample is a blood or serum sample. In some embodiments, the biological sample is a urine sample.

Methods for determining protein levels in biological samples are well known to those of skill in the art and include, but are not limited to, western blot, protein array, mass spectrometry, ELISA, and ELISPOT assays. Other suitable assays for the detection of any of the proteins listed above are well known to those of skill in the art and the invention is not limited in this respect.

The proteins provided herein as biomarkers useful for the diagnosis of FSGS are well known in the art and representative sequences of these proteins can be retrieved from public databases. Exemplary, representative sequences are provided below. The sequences are exemplary and not limiting. Those of skill in the art will be able to ascertain additional sequences, such as splice variants, posttranslationally modified versions, and naturally occurring mutated versions, of the biomarker proteins provided. For example, where precursors are listed, the assessment of the level of the processed, mature protein is also useful for the diagnosis of FSGS, and vice versa.

alpha 1 antitrypsin
>gi|50363221|ref|NP_001002235.1| alpha-l-
antitrypsin precursor [Homo sapiens]
MPSSVSWGILLLAGLCCLVPVSLAEDPQGDAAQKTDTSHHDQDHPTFNKI
TPNLAEFAFSLYRQLAHQSNSTNIFFSPVSIATAFAMLSLGTKADTHDEI
LEGLNFNLTEIPEAQIHEGFQELLRTLNQPDSQLQLTTGNGLFLSEGLKL
VDKFLEDVKKLYHSEAFTVNFGDTEEAKKQINDYVEKGTQGKIVDLVKEL
DRDTVFALVNYIFFKGKWERPFEVKDTEEEDFHVDQVTTVKVPMMKRLGM
FNIQHCKKLSSWVLLMKYLGNATAIFFLPDEGKLQHLENELTHDIITKFL
ENEDRRSASLHLPKLSITGTYDLKSVLGQLGITKVFSNGADLSGVTEEAP
LKLSKAVHKAVLTIDEKGTEAAGAMFLEAIPMSIPPEVKFNKPFVFLMIE
QNTKSPLFMGKVVNPTQK
beta-2 glycoprotein
>gi|153266841|ref|NP_000033.2| beta-2-
glycoprotein 1 precursor [Homo sapiens]
MISPVLILFSSFLCHVAIAGRTCPKPDDLPFSTVVPLKTFYEPGEEITYS
CKPGYVSRGGMRKFICPLTGLWPINTLKCTPRVCPFAGILENGAVRYTTF
EYPNTISFSCNTGFYLNGADSAKCTEEGKWSPELPVCAPIICPPPSIPTF
ATLRVYKPSAGNNSLYRDTAVFECLPQHAMFGNDTITCTTHGNWTKLPEC
REVKCPFPSRPDNGFVNYPAKPTLYYKDKATFGCHDGYSLDGPEEIECTK
LGNWSAMPSCKASCKVPVKKATVVYQGERVKIQEKFKNGMLHGDKVSFFC
KNKEKKCSYTEDAQCIDGTIEVPKCFKEHSSLAFWKTDASDVKPC
alpha-1 microglobulin
>gi|4502067|ref|NP_001624.1| protein AMBP
preproprotein [Homo sapiens]
MRSLGALLLLLSACLAVSAGPVPTPPDNIQVQENFNISRIYGKWYNLAIG
STCPWLKKIMDRMTVSTLVLGEGATEAEISMTSTRWRKGVCEETSGAYEK
TDTDGKFLYHKSKWNITMESYVVHTNYDEYAIFLTKKFSRHHGPTITAKL
YGRAPQLRETLLQDFRVVAQGVGIPEDSIFTMADRGECVPGEQEPEPILI
PRVRRAVLPQEEEGSGGGQLVTEVTKKEDSCQLGYSAGPCMGMTSRYFYN
GTSMACETFQYGGCMGNGNNEVTEKECLQTCRTVAACNLPIVRGPCRAFI
QLWAFDAVKGKCVLFPYGGCQGNGNKFYSEKECREYCGVPGDGDEELLRF
SN
transthyretin
>gi|4507725|ref|NP_000362.1| transthyretin
precursor [Homo sapiens]
MASHRLLLLCLAGLVFVSEAGPTGTGESKCPLMVKVLDAVRGSPAINVAV
HVFRKAADDTWEPFASGKTSESGELHGLTTEEEFVEGIYKVEIDTKSYWK
ALGISPFHEHAEVVFTANDSGPRRYTIAALLSPYSYSTTAVVTNPKE
apolipoprotein E
>gi|178853|gb|AAB59397.1| apolipoprotein E
[Homo sapiens]
MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVRGRLVQYRGEVQAMLGQSTEE
LRVRLASHLRKLRKRLLRDADDLQKRLAVYQAGAREGAERGLSAIRERLG
PLVEQGRVRAATVGSLAGQPLQERAQAWGERLRARMEEMGSRTRDRLDEV
KEQVAEVRAKLEEQAQQIRLQAEAFQARLKSWFEPLVEDMQRQWAGLVEK
VQAAVGTSAAPVPSDNH
apolipoprotein A IV
>gi|71773110|ref|NP_000473.2| apolipoprotein
A-IV precursor [Homo sapiens]
MFLKAVVLTLALVAVAGARAEVSADQVATVMWDYFSQLSNNAKEAVEHLQ
KSELTQQLNALFQDKLGEVNTYAGDLQKKLVPFATELHERLAKDSEKLKE
EIGKELEELRARLLPHANEVSQKIGDNLRELQQRLEPYADQLRTQVSTQA
EQLRRQLTPYAQRMERVLRENADSLQASLRPHADELKAKIDQNVEELKGR
LTPYADEFKVKIDQTVEELRRSLAPYAQDTQEKLNHQLEGLTFQMKKNAE
ELKARISASAEELRQRLAPLAEDVRGNLRGNTEGLQKSLAELGGHLDQQV
EEFRRRVEPYGENFNKALVQQMEQLRQKLGPHAGDVEGHLSFLEKDLRDK
VNSFFSTFKEKESQDKTLSLPELEQQQEQQQEQQQEQVQMLAPLES
serotransferrin
>gi|4557871|ref|NP_001054.1| serotransferrin
precursor [Homo sapiens]
MRLAVGALLVCAVLGLCLAVPDKTVRWCAVSEHEATKCQSFRDHMKSVIP
SDGPSVACVKKASYLDCIRAIAANEADAVTLDAGLVYDAYLAPNNLKPVV
AEFYGSKEDPQTFYYAVAVVKKDSGFQMNQLRGKKSCHTGLGRSAGWNIP
IGLLYCDLPEPRKPLEKAVANFFSGSCAPCADGTDFPQLCQLCPGCGCST
LNQYFGYSGAFKCLKDGAGDVAFVKHSTIFENLANKADRDQYELLCLDNT
RKPVDEYKDCHLAQVPSHTVVARSMGGKEDLIWELLNQAQEHFGKDKSKE
FQLFSSPHGKDLLFKDSAHGFLKVPPRMDAKMYLGYEYVTAIRNLREGTC
PEAPTDECKPVKWCALSHHERLKCDEWSVNSVGKIECVSAETTEDCIAKI
MNGEADAMSLDGGFVYIAGKCGLVPVLAENYNKSDNCEDTPEAGYFAVAV
VKKSASDLTWDNLKGKKSCHTAVGRTAGWNIPMGLLYNKINHCRFDEFFS
EGCAPGSKKDSSLCKLCMGSGLNLCEPNNKEGYYGYTGAFRCLVEKGDVA
FVKHQTVPQNTGGKNPDPWAKNLNEKDYELLCLDGTRKPVEEYANCHLAR
APNHAVVTRKDKEACVHKILRQQQHLFGSNVTDCSGNFCLFRSETKDLLF
RDDTVCLAKLHDRNTYEKYLGEEYVKAVGNLRKCSTSSLLEACTFRRP
Vitamin D binding protein
>gi|32483410|ref|NP_000574.2| vitamin
D-binding protein precursor [Homo sapiens]
MKRVLVLLLAVAFGHALERGRDYEKNKVCKEFSHLGKEDFTSLSLVLYSR
KFPSGTFEQVSQLVKEVVSLTEACCAEGADPDCYDTRTSALSAKSCESNS
PFPVHPGTAECCTKEGLERKLCMAALKHQPQEFPTYVEPTNDEICEAFRK
DPKEYANQFMWEYSTNYGQAPLSLLVSYTKSYLSMVGSCCTSASPTVCFL
KERLQLKHLSLLTTLSNRVCSQYAAYGEKKSRLSNLIKLAQKVPTADLED
VLPLAEDITNILSKCCESASEDCMAKELPEHTVKLCDNLSTKNSKFEDCC
QEKTAMDVFVCTYFMPAAQLPELPDVELPTNKDVCDPGNTKVMDKYTFEL
SRRTHLPEVFLSKVLEPTLKSLGECCDVEDSTTCFNAKGPLLKKELSSFI
DKGQELCADYSENTFTEYKKKLAERLKAKLPDATPTELAKLVNKHSDFAS
NCCSINSPPLYCDSEIDAELKNIL

In some embodiments, a method for choosing a course of treatment is provided, comprising diagnosing a subject as having or being at risk of developing FSGS, and choosing a course of treatment, for example, of clinical treatment, of the subject based on the subject being diagnosed with FSGS or a risk of developing FSGS.

Treatments appropriate for a subject having FSGS are well known to those of skill in the art and include, for example, administration of corticosteroids (see, e.g., Semin Nephrol. 2000; 20:309-317), cyclophosphamide (see, e.g., Semin Nephrol. 2000; 20:309-317), cyclosporine and/or low-dose prednisone (see, e.g., Kidney Int. 1999; 56:2220-2226), tacrolimus, alkylating agents (see, e.g., Am J Kidney Dis. 2004; 43:10-18), mycophenolate mofetil (see, e.g., Clin Nephrol. 2004; 62:405-411, Kidney Int. 2002; 61:1098-1114), or sirolimus (see, e.g., Clin J Am Soc Nephrol. 2006; 1:109-116).

In some embodiments, the subject is a human subject. In some embodiments, the subject is a subject from a family at least one member of which is affected by FSGS. In some embodiments, the subject is a subject that is not diagnosed with a renal cystic ciliopathy, for example, a subject that is not diagnosed with nephronophthisis. In some embodiments, the subject does not exhibit signs of a ciliopathy.

These and other aspects of the invention are illustrated by the following non-limiting examples.

### EXAMPLES

### Example 1

### Materials and Methods

### Subjects, Clinical Data and DNA Samples:

Subjects were enrolled in this study after obtaining informed consent in accordance with a human subjects protocol approved by the King Faisal Specialist Hospital & Research Centre Institutional Ethics Research Committee and the Human Research Committee at the Brigham and Women's Hospital. These families were recruited as part of an ongoing study of the genetic basis of kidney disease through referral by clinicians. Clinical and family history information was obtained and medical records, kidney biopsies, and biopsy reports were reviewed when available. DNA was extracted from peripheral blood using the Gentra Puregene Blood Kit (Qiagen, Germany, catalog number D-40K) according to the manufacturer protocol.

### DNA Sequencing and Genotyping:

PCR-amplified segments containing coding sequences and flanking splice sites of 4 known FSGS/NS genes (NPHS2, ACTN4, TRPC6 and INF2) were re-sequenced using standard Sanger sequencing methodology on an Applied Biosystems 3730x1DNA sequencer (Polymorphics Technology Inc: http://www.polymorphicdna.com/). The sequencing was done on six affected subjects and two unaffected subjects from FAM001 and FAM012.

In both family 1 (FAM001) and family 12 (FAM012), a genome-wide analysis was performed using 250K Affymetrix SNP following the manufacturer protocol at the Microarray Core at the Dana-Farber Cancer Institute. Genotyping was done using genomic DNA samples from 11 informative family members. After sample hybridization the arrays were scanned and the generated data were analyzed to identify homozygous runs in the samples using a Hidden Markov Model with two hidden states. A Markov chain was modeled with two states, heterozygous run and homozygous run, along the genome of every sample, with one step for every locus genotyped with the Affymetrix platform. Following the notation in R. Durbin et al., Biological sequence analysis: Probabilistic models of proteins and nucleic acids (Cambridge Univ Pr, 1998), the emission probabilities eⱼₖ(t) were set for j=0, 1 and k=0 . . . 2, where j is 0 for a heterozygous run and l for a homozygous run, k is the number of non reference alleles observed at locus t, and p(t) is the frequency of the reference allele at locus t, as

$$e = ( \begin{matrix}{p}^{2} & 2 p ( 1 - p ) & {( 1 - p )}^{2} \\ p ( 1 - δ ) & δ & ( 1 - p ) ( 1 - δ )\end{matrix} )$$

where δ˜2⁻⁸ is a parameter that allows heterozygous loci to be exceptionally recognized as being emitted by homozygous run states to allow for genotype error and where for clarity the variable t indicating the locus was dropped. Transmission probabilities α_{i,j} were set for i,j=0, 1 as

$$a = ( \begin{matrix}1 - ɛ & ɛ \\ ɛ & 1 - ɛ\end{matrix} )$$

where ε˜2⁻⁴⁰ is chosen small enough to avoid short false homozygous run positives. This model, though simple, was able to efficiently identify long homozygous runs in this high density of SNP data.

### Whole Exome Capture and Sequencing:

Whole exome capture and sequencing was performed as described in Choi M, Scholl U I, Ji W, Liu T, et al. Genetic diagnosis by whole exome capture and massively parallel DNA sequencing. Proceedings of the National Academy of Sciences of the United States of America 2009; 106: 19096-19101. In brief, genomic DNA was captured on a NimbleGen 2.1M human exome array following the manufacturer's protocols (Roche/NimbleGen) at the W. M. Keck Facility at Yale University. DNA was sheared by sonication, ligated to adaptors, and fractionated by agarose gel electrophoresis. Fragments of the desired size were excised and DNA was extracted and amplified by ligation mediated PCR. This was followed by hybridization to the capture array. The array was washed and bound genomic DNA was eluted and amplified by ligation-mediated PCR. The resulting fragments were purified and subjected to DNA sequencing on two lanes of the Illumina platform. 36,792,066 72 reads were sequenced and these reads were aligned using the Burrows-Wheeler Transform algorithm implemented in bwa (Li H and Durbin R, Bioinformatics 2009). All reads were aligned using the ‘aln’ command of bwa with the exception to 723,020 reads, which were reprocessed using the ‘bwasw’ command of bwa, which reduced unaligned reads to 210,851 reads.

### Deletion Assay:

Primers were designed from three genomic regions, one inside the deletion and two in the flanking regions. Three pairs of primers were designed for each region using the publicly available tool NCBI/Primer-Blast (www.ncbi.nlm.nih.gov/tools/primer-blast). Primers are shown in Table 4. 10 ng of genomic DNA from all subjects in the two families and subject 41 was used for PCR. Thirty-cycle PCR was carried after denaturation step at 95° C. (5 minutes). Cycle conditions are 95° C. for thirty seconds, 56° C. for thirty seconds, and 72° C. for thirty seconds. The Thirty-cycle PCR was then followed by a final step of 72° C. for seven minutes. Resulting PCR fragments were then visualized with ethidium bromide agarose gel.

TABLE 4
List of primers used in the PCR deletion assay

| Primer | Genomic Region | Forward | Reverse |
|---|---|---|---|
| A | Left Flank | GCGCCTGTGCATCAGTGGTAATA | CAGGGGCTGACAGCATGGC |
| B | | ACTCACCGAGGCTGCCGAAT | TGTGGGGGTGGTGTTTTCCCA |
| C | | CCACCCAGGAAGAGCCATGC | TGGCGAGACGACAGCGAGAT |
| D | Inside deletion | GTGCCGCACTCACCAGCTC | GCTGCAACAGTATATTGCATGCTC |
| E | | ATGGGGGTAGGCGTGTGGAG | CATTTGTGCTCATGCAGCCTGG |
| F | | TGCACAGCCTGACCATACCTG | TGTGCAACTAACTCAGGATTCCACT |
| G | Right flank | ACAGCACCAATGCTAATACTCACCT | GCTGGCGCTGCTGTCTCTAAC |
| H | | CAGGTCAATCAGTGCCAAGCCA | GCGTGGCTCTGCTCTGCATT |
| I | | AGAGATGACAAGACCTTGAGGCA | GCTCACCTCGTTTCTCATCTCCTTG |

### Example 2

### Clinical and Histological Findings

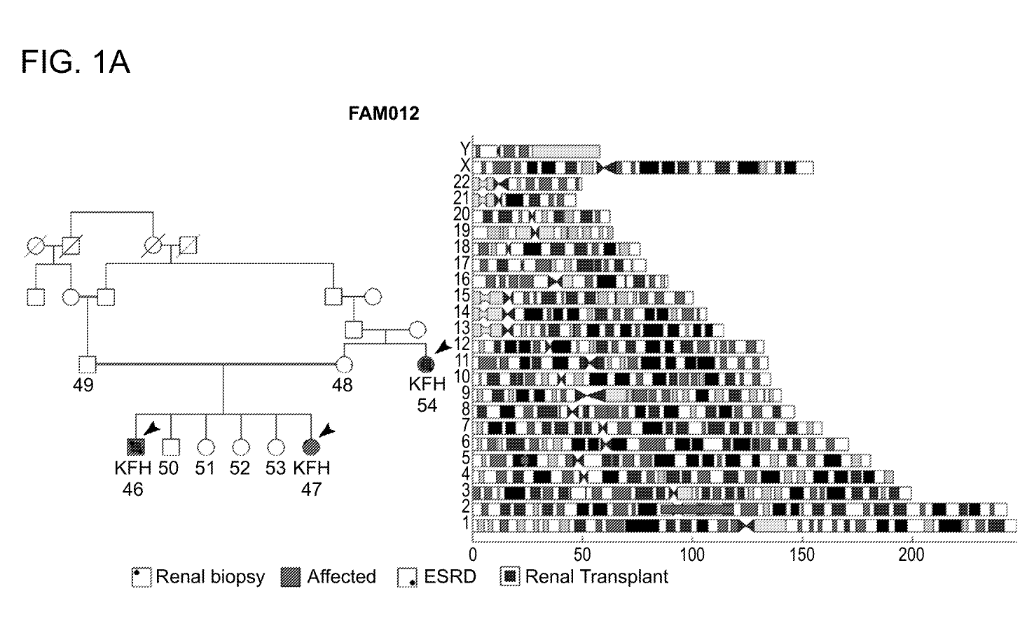

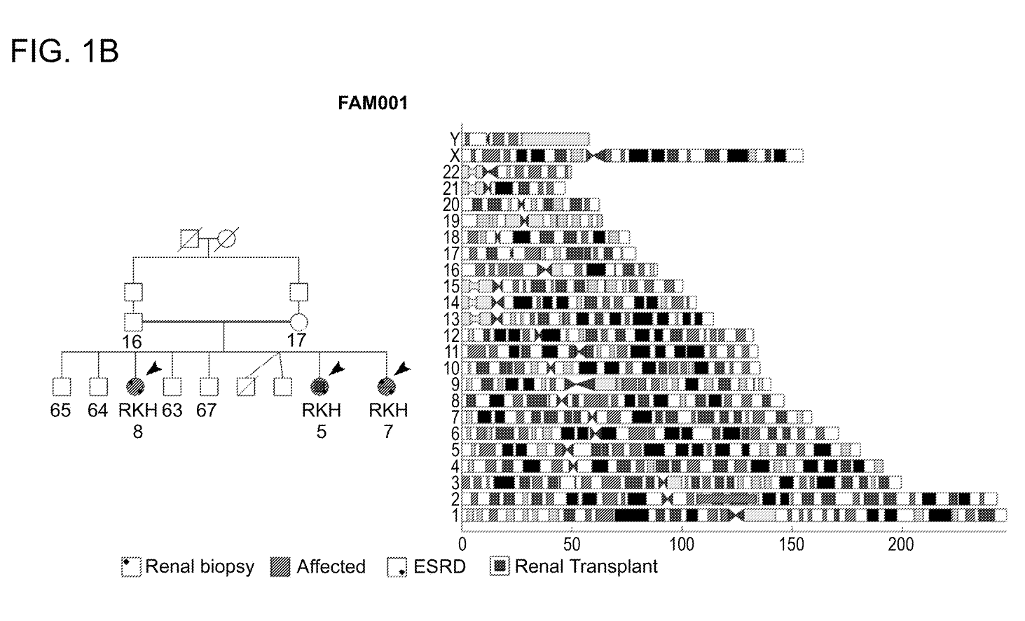

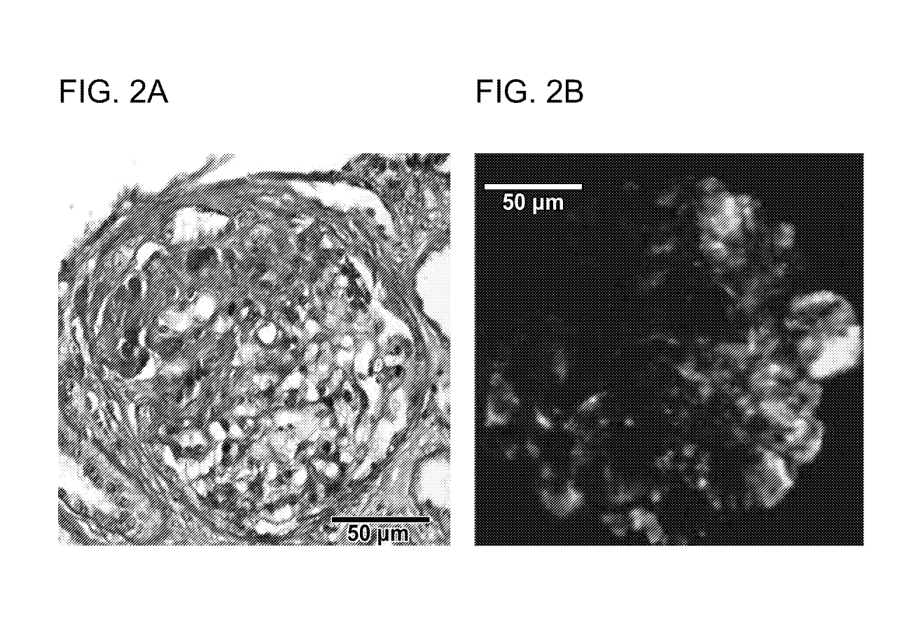

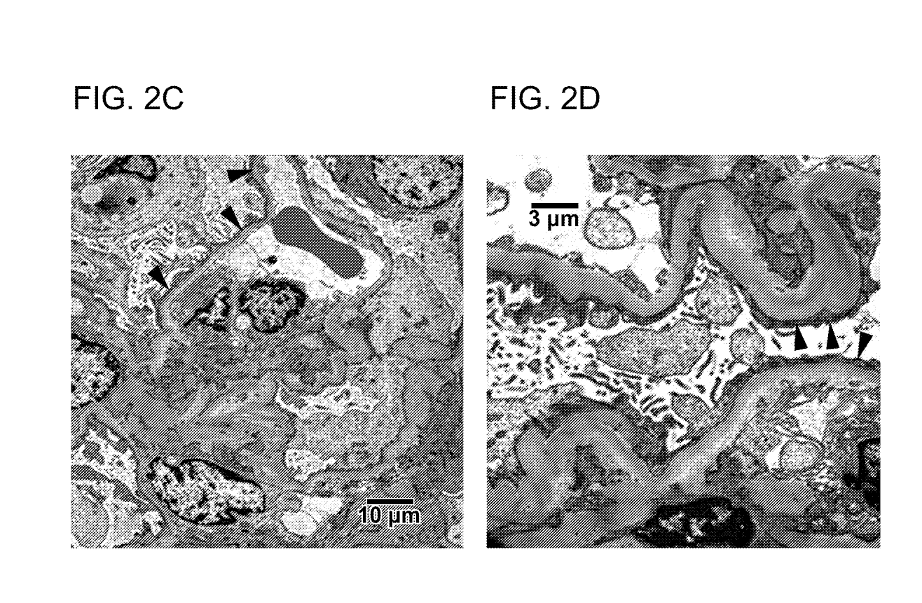

This study describes two consanguineous Saudi Arabian families with a family history of nephrotic range proteinuria range, end-stage renal disease (ESRD), and differing histologic findings on kidney biopsy. The numbers of affected and unaffected subjects correspond to what is shown on the pedigrees in FIG. 1. The first family in this study (FAM012) has a history of kidney failure, first manifested as nephrotic range proteinuria (>1.5 gm/24 hrs) which was observed in the aunt (KFH-54) at an unconfirmed age. The aunt's nephew (KFH-46) and niece (KFH-47) also developed nephrotic range proteinuria (>1.5 gm/24 hrs) both 9 years of age. It is unknown if other features of nephrotic syndrome were present. A kidney biopsy was performed on individual KFH-46. A copy of the report and selected light and electron microscopic images were obtained for review. Per report, the biopsy showed global glomerulosclerosis in some of 12 glomeruli. It also showed segmental glomerulosclerosis in some of 12 glomeruli, without evidence of vascular disease (FIG. 2a). Immunofluorescence studies revealed focal staining for C3 and IgM in the sclerosed glomerular segments (FIG. 2b). Electron microscopic images provided demonstrated areas of extensive foot process effacement of visceral epithelial cells, microvillous change of the apical surface of these cells, and glomerular basement membrane wrinkling, changes consistent with focal segmental glomerulosclerosis (FSGS) (FIGS. 2c+d). The true nature of FSGS (primary vs. secondary) in this patient could not be determined based on the limited material provided. The affected subject KFH-46 was treated with steroids and other alternative therapy and showed no response and progressed to ESRD within a few years of initial diagnosis. Both the aunt (KFH-54) and the nephew (KFH-46) received a cadaveric kidney transplant and are doing well as evident by normal protein/creatinine levels up to date.

The second family (FAM001) is of an un-related tribal ancestry to FAM012. FAM001 has three siblings with ESRD, RKH-5, RKH-7 and RKH-8; RKH-8 is a female who presented at 21 years of age with renal failure of unknown cause without known history of hypertension, haematuria, or proteinuria. Kidney biopsy was performed on affected RKH-8, and a copy of the report and selected light microscopic images were obtained for review. Per report, sections showed nine glomeruli, seven of which were globally sclerosed, and two of which appeared unremarkable on light microscopy. Additional findings included marked interstitial fibrosis, patchy chronic inflammation and tubular atrophy, as well as marked medial hypertrophy of small arteries, consistent with vascular sclerosing disease. Electron micrographs were not provided. Taken together, the changes described can be seen as a non-specific response to any type of chronic renal injury, and are most commonly associated with chronic vascular disease or diabetes. RKH-7 is a female who presented at 15 years of age with mild renal impairment and a strong family history of chronic renal failure. Kidney biopsy was obtained from RKH-7. A copy of the report and selected light microscopic images were obtained for review. Per report, sections showed 18 glomeruli, four of which were globally sclerosed, the remaining 14 revealed no significant pathologic changes on light microscopy. Additional findings seen on images provided included focal interstitial fibrosis, tubular atrophy, and marked vascular changes, potentially representing vascular sclerosing disease in the context of chronic hypertension or diabetes. Immunofluorescence studies were reportedly negative for C3 and C1q. Electron micrographs were not provided for review. Based on the limited material received for review, the changes found are non-specific and do not show characteristic features of a distinct disease entity. Serum creatinine level was 217 μmol/L for RKH-7 and 283 μmol/L for RKH-8. RKH-5 in this family presented at 12 years of age with severe kidney failure, and no native kidney biopsy was obtained. The affected subject RKH-5 received a kidney transplant and has been doing well to date. The transplant kidney biopsy showed normal glomeruli and no significant pathology.

The clinical parameters for the two families are shown on Table 1 and representative of the histopathology findings is shown in FIG. 2. From the history, it was inferred that the two families likely had a recessive genetic trait. After a genetic screen for known nephrotic syndrome and FSGS genes, a Genome-wide SNP genotyping was performed as described below.

TABLE 1
clinical parameters

| | | | Age at | | | | | |
|---|---|---|---|---|---|---|---|---|
| | | | Diagnosis | History for | Kidney Biopsy | Serum | Presense of | Patient Current |
| | Number | Gender | (Current) | Proteinuria* | observations | creatinin | kidney cysts | Status |
| FAM012 | KFH-46 | Male | 9 (16) | 2 gm per 24 hrs | Changes | not known | NO | Post renal |
| | | | | | consistent with | | | transplant |
| | | | | | FSGS | | | |
| | KFH-47 | Female | 9 (10) | 2 gm per 24 hrs | not known | not known | NO | ESRD, on |
| | | | | | | | | Dialysis |
| | KFH-48 | Female | unaffected | negative | n/a | n/a | n/a | n/a |
| | KFH-49 | Male | unaffected | negative | n/a | n/a | n/a | n/a |
| | KFH-50 | Male | unaffected | negative | n/a | n/a | n/a | n/a |
| | KFH-51 | Female | unaffected | negative | n/a | n/a | n/a | n/a |
| | KFH-52 | Female | unaffected | negative | n/a | n/a | n/a | n/a |
| | KFH-53 | Female | unaffected | negative | n/a | n/a | n/a | n/a |
| | KFH-54 | Female | not known | >40 mg/m2/hr | not known | not known | n/a | Post renal |
| | | | | | | | | transplant |
| FAM001 | RKH-5 | Female | 12 (22) | not known | n/a | 149 μmol/L | NO | ESRD, post |
| | | | | | | | | renal transplant |
| | RKH-7 | Female | 15 (19) | not known | None-specific focal | 217 μmol/L | NO | ESRD, on |
| | | | | | global | | | Dialysis |
| | | | | | glomerulosclerosis. | | | |
| | | | | | Interstitial | | | |
| | | | | | fibrosis and | | | |
| | | | | | vascular changes | | | |
| | RKH-8 | Female | 21 (34) | not known | None-specific focal | 283 μmol/L | ? | ESRD, on |
| | | | | | global | | | Dialysis |
| | | | | | glomerulosclerosis. | | | |
| | | | | | Interstitial | | | |
| | | | | | fibrosis and | | | |
| | | | | | vascular changes | | | |
| | RKH-16 | Male | unknown | unknown | n/a | n/a | n/a | n/a |
| | RKH-17 | Female | unaffected | negative (dip stick) | n/a | n/a | n/a | n/a |
| | RKH-63 | Male | unaffected | negative (dip stick) | n/a | n/a | n/a | n/a |
| | RKH-64 | Male | unaffected | negative (dip stick) | n/a | n/a | n/a | n/a |
| | RKH-65 | Male | unaffected | negative (dip stick) | n/a | n/a | n/a | n/a |
| | RKH-67 | Male | unaffected | 30 (dip stick) | n/a | n/a | n/a | n/a |

*Proteinuria at diagnosis using any of these analysis [spot urine with protein/creatinin. 24 hr protein in urine. spot urine with albumine/creatinin. OR dip-stick]
n/a = not applicable

### Candidate NS/FSGS Gene Screening

Two consanguineous families affected with renal failure accompanied with proteinuria of the nephrotic range and biopsies of mixed findings of focal, segmental and glomerulosclerosis FSGS and global glomerulosclerosis were identified and characterized. To identify the genetic defect responsible for this phenotype in these two families, a candidate gene approach was first used by performing mutational analysis for genes known to be associated with nephrotic syndrome NS and FSGS. Sanger dideoxy DNA sequencing methodology was used to resequence PCR-amplified segments containing coding sequences and flanking splice sites of 4 known FSGS/NS genes (NPHS2, ACTN4, TRPC6 and INF2) in eight subjects (six affected and 2 unaffected subjects from both family FAM001 and family FAM012). Mutational analysis using this method showed no sequence variants in the sequenced genes in these two families (data not shown).

### Whole-Genome SNP Analysis and Homozygosity Mapping

Since no known FSGS/NS genes were found mutated by sequencing, high-resolution SNP analysis (Affymetrix GeneChip Genome-Wide Human Array 250K) was performed with the aim to identify the genetic variant responsible for the disease in these two families. The initial genotyping was performed on the three affected subjects and the two unaffected parents from the consanguineous family FAM012 (FIG. 1a). In this initial whole-genome genotyping scan, assuming recessive inheritance, large identity by descent (IBD) homozygous regions (homozygous runs) were searched for in the patients of the first family (FAM012). One homozygous run was found in the three affected subjects of this family (KFH-46, KFH-47 and the aunt KFH-54), giving first evidence for genetic linkage. The homozygous run is on chromosome 2q13, and falls between two SNP markers, rs757139 (genomic position 85,539,243) and rs1437432 (genomic position 117,490,034), and is approximately 31 Mb in size.

An independent genotyping experiment was performed on the second consanguineous family (FAM001). The three affected subjects (RKH-7, RKH-5 and RKH-8), the two unaffected parents, and one unaffected sibling were genotyped from this family. A homozygous run was detected in the affected subjects RKH-7, RKH-5 and RKH-8 (FIG. 1 b). This homozygous run falls between rs10496407 (genomic position 106,786,575) and rs67212013 (genomic position 133,608,192), and is approximately 27 Mb in size.

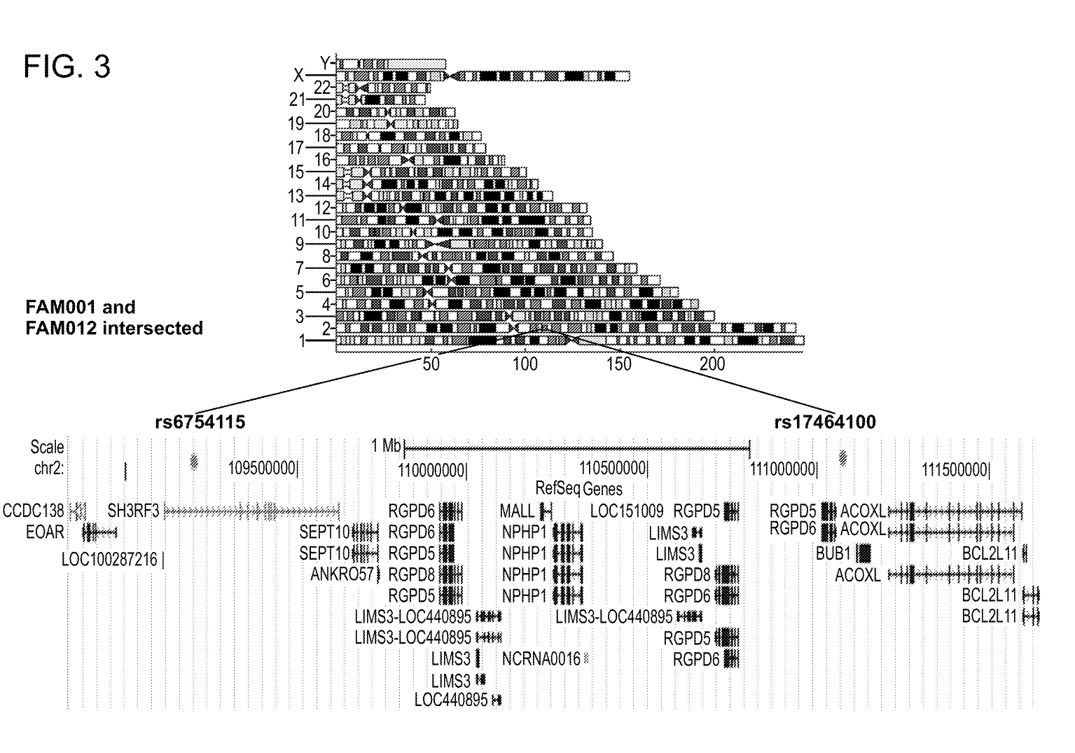

When the affected subjects from the two unrelated families were compared with each other, they were found to share the same homozygous run between rs67541 15 (genomic position 109,328,776) and rs17464100 (genomic position 111,284,252) reducing the critical homozygous interval to ˜2 Mb that is shared in the affected subjects from the two families (FIG. 3). The ˜2 Mb homozygous run contains a number of known genes including the septin 10 isoform 2 (SEPT10), RANBP2-like and GRIP domain containing 5 isoforms (RGPD1, RGPD5, and RGPD6), LIM and senescent cell antigen-like domains 3 (LIMS3), the T-cell differentiation protein-like mal (MALL), the nephrocystin 1 isoform 3 (NPHP1), budding uninhibited by benzimidazoles 1 (BUB1), the SH3 domain containing ring finger 3 (SH3RF3), and a number of hypothetical genes (FIG. 3). A whole exome capture was undertaken utilizing the Roche/NimbleGen 2.1M Human Exome Array, followed by massively parallel sequencing using the Illumina platform to identify the possible genetic defects associated with clinical and pathological findings in these two consanguineous families.

### Coupling of NimbleGen Whole-Exome Capture to Illumina Sequencing

Given the diagnostic uncertainty and for rapid genetic diagnosis to identify the causal genetic defect in these two consanguineous families, homozygosity mapping was coupled with an approach to sequence complete coding regions of the genome (exome). The protocols for whole exome capture and sequencing using the Roche/NimbleGen 2.1M Human Exome Array were described by Choi M et al, PNAS, 2010 and outlined in brief in the methods section of this paper. Genomic DNA from affected subject RKH-5 in FAM001 was captured and sequenced. The resulting sequence data were filtered and aligned to the reference human genome (hg18) as described in methods. A total of 36,792,066 74 bp-reads were generated and 99.4% aligned to the human reference sequence; 41% of the total bases mapped to the targeted bases with mean coverage of 32×.

In the exome capture and sequencing for RKH-5, a total of 8147 exomic single nucleotide polymorphism (SNP) variations were found from the reference sequence; 3384 exomic SNPs were homozygous and 4863 SNPs were heterozygous. A total of 183 exomic copy number polymorphisms (CNPs; e.g., insertions or deletions) were also detected in this sample; 34 of these CNPs were homozygous and 149 CNPs were heterozygous. After filtering the exomic SNPs against the dbSNPs-130 (www.ncbi.nlm.nih.gov/projects/SNP/) a total of 808 SNPs were found that are novel in RKH-5; 31 were homozygous and 777 were heterozygous (Table 2). When filtered against dbSNP-130 and the search was restricted in the largest homozygous run (chr2:106,764,546-133,600,658) in FAM001 a total number of 2 homozygous SNPs was found, 1 homozygous CNPs and as expected Zero heterozygous SNPs and CNPs.

TABLE 2
Summary Statistics for whole exome capture on sample RKH-5
Table 2: Summary Statistics for whole exome capture on Sample RKH-5

| | | | | | |
|---|---|---|---|---|---|
| Total exomic | 8147 | Total Exomic | 183 | Total filtered* | 808 |
| SNPs | | CNPs | | exomic SNPs | |
| Homozygous | 3284 | Homozygous | 34 | Homozygous | 31 |
| SNPs | | CNPs | | SNPs | |
| Heterozygous | 4863 | Heterozygous | 149 | Heterozygous | 111 |
| SNPs | | CNPs | | SNPs | |

*Filtering using dbSNP 130 (http://www.ncbi.nlm.nih.gov/projects/SNP/)
SNP = Single Nucleotide Polymorphisms. CNP = Copy Number Polymorphisms

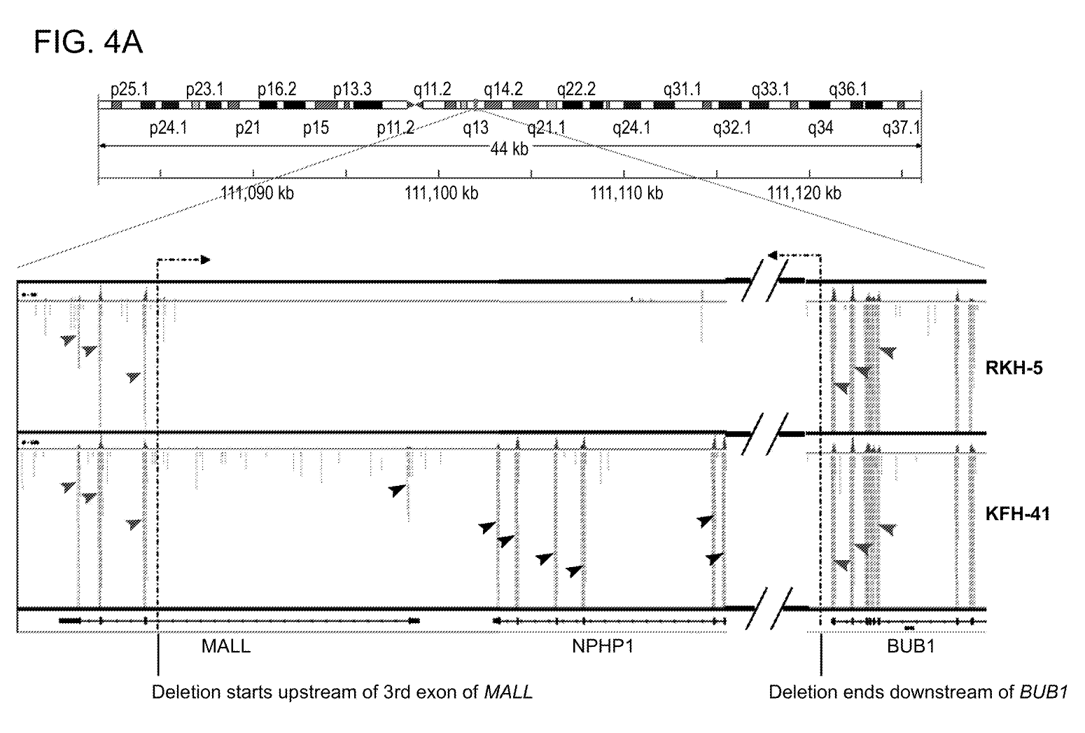

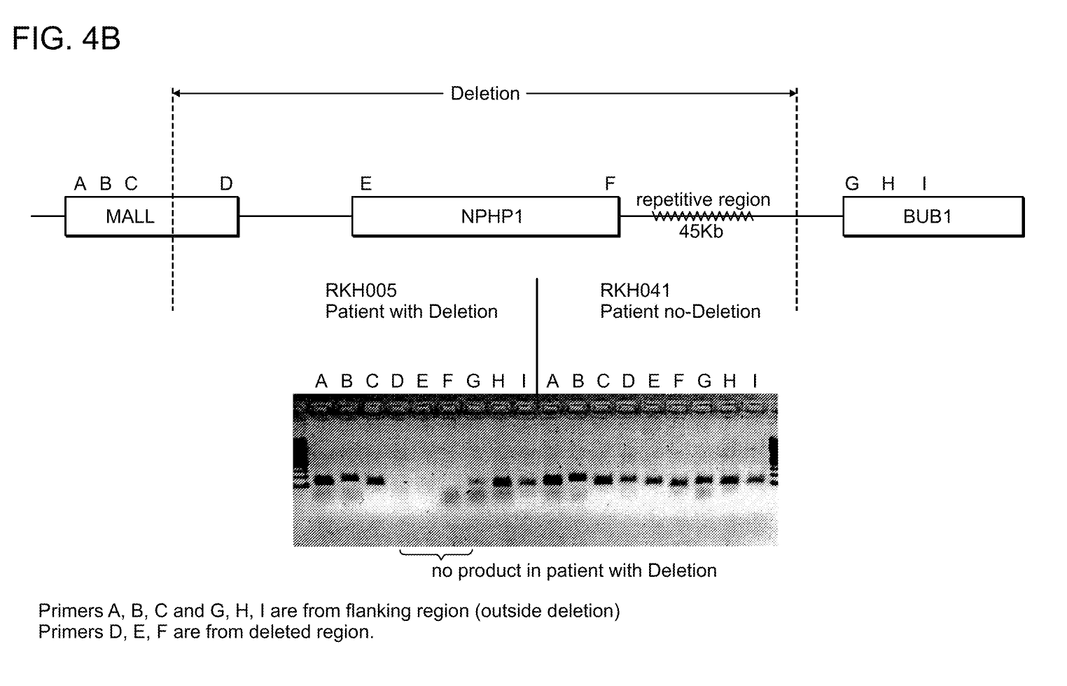

A homozygous disease-causing mutation was anticipated within the ˜2 Mb homozygous run that is shared between the two consanguineous families. Consequently, Informative mutations in this segment were sought directly. A major structural variation was observed in the exome sequence data in subject RKH005 when it was compared to the exome sequence data from another subject using the same platform and technology (subject KFH041). Subject 41 is from a different family that did not share this homozygous run whose genomic DNA was analyzed by whole exome capture and sequencing using the Roche/NimbleGen 2.1M Human Exome Array (mean exome coverage of 32×). In RKH-5 sample, exome capture and sequencing failed (zero coverage) in twenty-one consecutive exons within the minimal homozygous run. These exons were captured and sequenced in subject 41 to a mean coverage per-exon of 75× (FIG. 4A). The failure to capture and sequence this region in RKH-5 was consistent with an observation that was found in the genotyping data for the two families. SNP detection efficiency between rs7575835 (genomic position 110,191,850) and rs7575835 (genomic position 110,337,635) were weak to no detection, which at first indicated possible homozygous deletion of the region (data not shown). The lack of sequence coverage in the same region in the exome data in RKH-5 further indicated the structural variant as a homozygous deletion in the region. This also prompted studying the mean sequencing coverage for 18 candidate FSGS genes and 9 NPHP genes in the exome data (Table 3). FSGS and NPHP genes had coverage of 38× in RKH-5 exome and 42× in KFH-46 exome. Five genes were systematically missed (zero coverage) in both RKH-5 and KFH-46 exomes, namely COQ2, PLCE1, INF2, NPHP4, and CEP290. The structural homozygous deletion encompasses 21 consecutive exons; all twenty exons of a gene named nephrocystin 1 isoform 3 (NPHP1) and the first exon of the adjacent gene named T-cell differentiation protein-like (MALL).

TABLE 3
Exome sequencing coverage in FSGS, NS
and NPHP genes in RKH-5 and KFH-41:

| Genomic Locus | | | | Coverage | |
|---|---|---|---|---|---|
| Chrmosome | Starts | Ends | Gene Symbol | RKH-5 | KFH-41 |
| 1 | 177786297 | 177811707 | NPHS2 | 35x | 35x |
| 1 | 202390571 | 202402088 | RENIN | 12x | 28x |
| 2 | 216985382 | 217056019 | SMARCAL1 | 33x | 44x |
| 3 | 49133551 | 49145603 | LAMB2 | 8x | 17x |
| 4 | 77298918 | 77354059 | SCARB2 | 71x | 71x |
| 4 | 84404001 | 84424988 | COQ2 | 0 | 0 |
| 6 | 47553484 | 47702955 | CD2AP | 90x | 71x |
| 6 | 107580454 | 107887472 | PDSS2 | 64x | 58x |
| 9 | 128416569 | 128503132 | LMX1B | 11x | 21x |
| 10 | 95743736 | 96078138 | PLCE1 | 0 | 0 |
| 11 | 100827505 | 100959869 | TRPC6 | 69x | 64x |
| 11 | 32266639 | 32467137 | WT1 | 23x | 29x |
| 14 | 104226988 | 104256992 | INF2 | 0 | 0 |
| 16 | 20251874 | 20271538 | UMOD | 17x | 30x |
| 17 | 71229111 | 71265494 | ITGB4 | 11x | 22x |
| 19 | 43830167 | 43904358 | ACTN4 | 20x | 38x |
| 19 | 41008114 | 41034579 | NPHS1 | 11x | 19x |
| 22 | 35007272 | 35113927 | MYH9 | 19x | 35x |
| 1 | 5845456 | 5975118 | NPHP4 | 0 | 0 |
| 2 | 110238202 | 110319928 | NPHP1 | 0 | 74x |
| 3 | 133882143 | 133923966 | NPHP3 | 77x | 67x |
| 3 | 122971299 | 123036616 | IQCB1 (NPHP5) | 80x | 64x |
| 9 | 101901331 | 102103247 | INVS (NPHP2) | 65x | 61x |
| 12 | 86966920 | 87060124 | CEP290 (NPHP6) | 0 | 0 |
| 16 | 4322225 | 4329599 | GLIS2 (NPHP7) | 6x | 14x |
| 16 | 52191318 | 52295272 | RPGRIP1L (NPHP8) | 101x | 84x |
| 17 | 24079958 | 24093911 | NEK8 (NPHP9) | 16x | 28x |

### NPHP1/MALL Deletion by PCR

To confirm the deletion, a PCR assay was designed to detect homozygous deletion of the NPHP1/MALL locus by means of absence of expected amplicon. Three primers were made for three consecutive loci in the left flanking region with good exome sequence coverage in both RKH-5 and subject 41, three primers were made for three consecutive loci inside the deleted region, and three primers were made for three consecutive loci on the right flanking region (primer sequences are found in methods). PCR was carried out for RKH-5 and subject 41 for all the amplicons simultaneously (FIG. 4B). For RKH-5 the PCR amplification gave the expected amplicons for the deletion flanking regions, however the amplification failed with the primers inside the deletion. Meantime all primers gave their expected amplicon in subject 41. This PCR deletion analysis demonstrated a homozygous deletion of NPHP1/MALL locus gene in RKH-5.

The same PCR analysis was carried out on all affected and unaffected subjects from FAM001 and FAM012 using the best amplified primers from the initial PCR (primers A, E, and H; see methods). The PCR deletion assay showed that the six affected from FAM001 (RKH-5, RKH-7, and RKH-8) and from FAM012 (patients KFH-46, KFH-47, and KFH-54) have the deletion in the NPHP1/MALL while their unaffected parents and siblings did not with the exception to subject 67 in FAM001 who has the deletion and is seemingly healthy at the time of the analysis. The same analysis was carried out eight sporadic patients with FSGS findings and four recessive families with FSGS findings all had the expected PCR products in the deletion locus and flanking regions.

### Example 3

### Novel Homozygous Runs for Recessive Familial Focal and Segmental Glomerulosclerosis (FSGS)

The overall incidence rate of End Stage Renal Failure (treated with hemodialysis) is 461 per million per year. The mortality rate amongst dialysis patients in 2008 was 12% (1338 patients out of 11,168 patients died). Currently, 3692 patients (17%) are on the waiting list for a kidney transplant.

One objective of the research leading to the current invention is the identification of genetic lesions associated with familial Focal and Segmental Glomerulosclerosis (FSGS). FSGS is a significant cause for end stage renal disease (ESRD), comprising up to 5% of adults and 20% of children. 300 FSGS patients were treated at the KFSHRC—Pediatrics Department between 2000-2008.

To better understand the underlying genetic factors contributing to FSGS, a genetic association study was performed. The genetic study described in this section included patients from multiple families affected by FSGS. Tables 5 and 6 shown the patient inclusion criteria and the analysis workflow. Table 7 shows the mutational analysis summary.

TABLE 5
Inclusion and classification criteria

| Inclusion Criteria | Classification of status |
|---|---|
| Each family required to have at | Affected: overt proteinuria, biopsy |
| least one individual with biopsy- | proven FSGS and ESRD (dialysis, |
| proven FSGS plus | transplantation) |
| Second member with one of | Probably affected: >100 mg/dl |
| the following: | proteinuria |
| Biopsy-proven FSGS | Unknown: 30-100 mg/dl proteinuria |
| ESRD (on dialysis) | Unaffected: negative proteinuria |
| Renal transplantation | (caveat penetrance, age-dependent |
| | phenotype) |

TABLE 6
Work Flow:

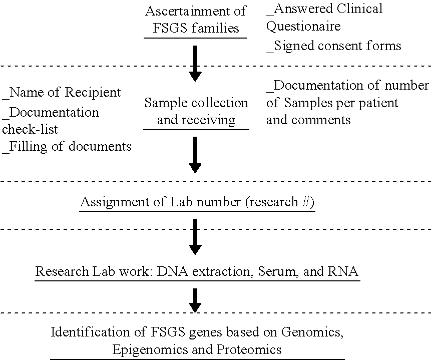

TABLE 7
Mutational Analysis

| | | | | | | | SNP | |
|---|---|---|---|---|---|---|---|---|
| | | | Proteinuria | | Age at Diagnosis | | (ACTN4, TRPC6, | |
| | Case Number | Gender | (mg/dL) | FSGS | (Current) | | INF2, NPHS2) | Patient Current Status |
| | RKH005 | Female | >100 | Yes | 12 | (22) | none | Dialysis |
| Family 1 | RKH007 | Female | >100 | Yes | ? | (19) | Ala > Val (NPHS2-E5), | Dialysis |
| | | | | | | | rs61747727 | |
| | RKH008 | Female | >100 | Yes | 21 | (34) | none | Dialysis |
| | RKH016 | Male | ? | No | unknown | (65) | n/a | n/a |
| | RKH017 | Female | −ve | No | unaffected | (60) | n/a | n/a |
| | RKH063 | Male | −ve | No | unaffected | | n/a | n/a |
| | RKH064 | Male | −ve | No | unaffected | | n/a | n/a |
| | RKH065 | Male | −ve | No | unaffected | | n/a | n/a |
| | RKH067 | Male | 30 | No | unaffected | | n/a | n/a |
| | RKH004 | Female | >100 | 48 | ? | (48) | none | Dialysis |
| Family 2 | RKH009 | Male | >100 | Yes | 19 | (28) | none | Dialysis |
| | RKH013 | Male | >100 | Yes | 19 | (27) | Ala > Val (TRPC6-E4), | Post Renal Transplant |
| | | | | | | | rs36111323 | |
| | RKH015 | Male | >100 | Yes | 18 | (25) | none | Post Renal Transplant |
| | RKH027 | Male | ? | No | unaffected | (50) | n/a | n/a |
| Family 15 | RKH028 | Male | ? | ? | ? | (75) | none | Dialysis |
| | KFH41 | Male | >100 | Yes | 26 | (18) | Glu > Gln (NPHS2-E5), | on treatment |
| | | | | | | | Novel SNP | |
| | KFH42 | Female | ? | No | Unknown | (?) | n/a | n/a |
| | KFH43 | Male | ? | No | Unknown | (?) | n/a | n/a |
| Family 12 | KFH44 | Male | > | No | unaffected | (9) | n/a | n/a |
| | KFH45 | Male | >100 | Yes | 5.5 | (7) | none | on treatment |
| | KFH046 | Male | >100 | Yes | 9 | (16) | none | Post Renal Transplant |
| | KFH047 | Female | >100 | Yes | 9 | (10) | none | Dialysis |
| | KFH048 | Female | −ve | No | unaffected | (?) | Glu > Gln (NPHS2), | n/a |
| | | | | | | | Novel | |
| | KFH049 | Male | −ve | No | unaffected | (?) | n/a | n/a |
| | KFH050 | Male | −ve | No | unaffected | (20) | n/a | n/a |
| | KFH051 | Female | −ve | No | unaffected | (23) | n/a | n/a |
| | KFH052 | Female | −ve | No | unaffected | (12) | n/a | n/a |
| | KFH053 | Female | −ve | No | unaffected | (3) | n/a | n/a |
| | KFH054 | Female | >100 | Yes | ? | (?) | none | Post Renal Transplant |

The results of this study demonstrate that mutations of Known FSGS genes account for only a small fraction of FSGS in this patient cohort. Further, no segregation with the disease of SNPs in FSGS known genes was observed. The pedigrees of 6 FSGS families studied in this cohort implied recessive inheritance. Finally, the selection of highly consanguineous FSGS families for genome-wide SNP association may reveal novel FSGS candidate regions/genes.

Shared Segment Mapping of Genotyping Data from Highly Dense SNP-Chips Identical by Descent (IBD):

Segments are identical by descent (IBD) in two or more individuals if they have been inherited from the same ancestral allele without recombination events inside the segments. Nature Genetics (2008) 40 (9): 1068-1075.

For the analysis of the study cohort, a recessive model was employed to detect long shared IBD DNA segments in affected individuals (homozygous runs). The model took into account the extent of inbreeding (consanguinity) in the individuals. In small families, recombination events were not modeled.

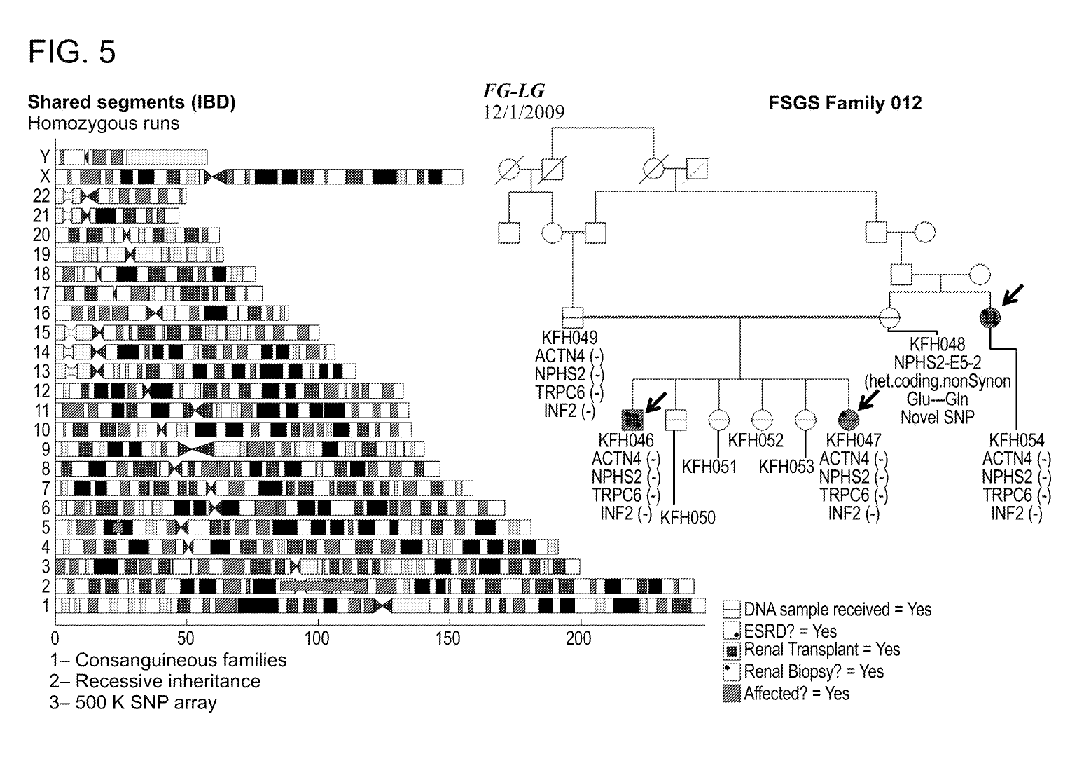

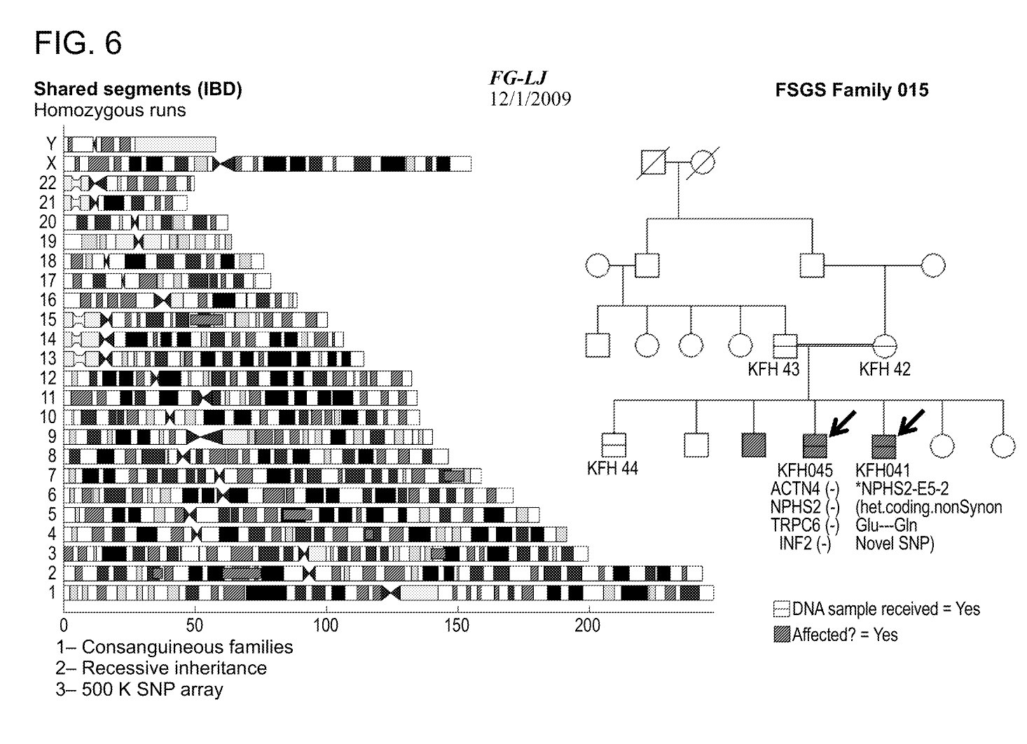

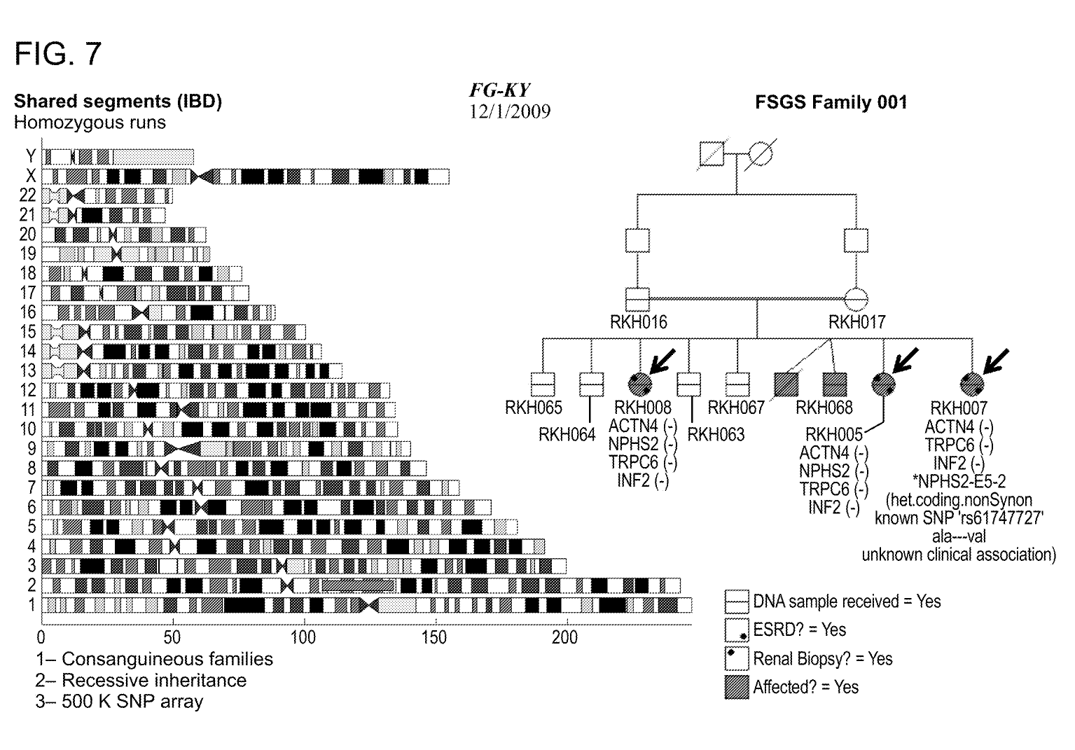

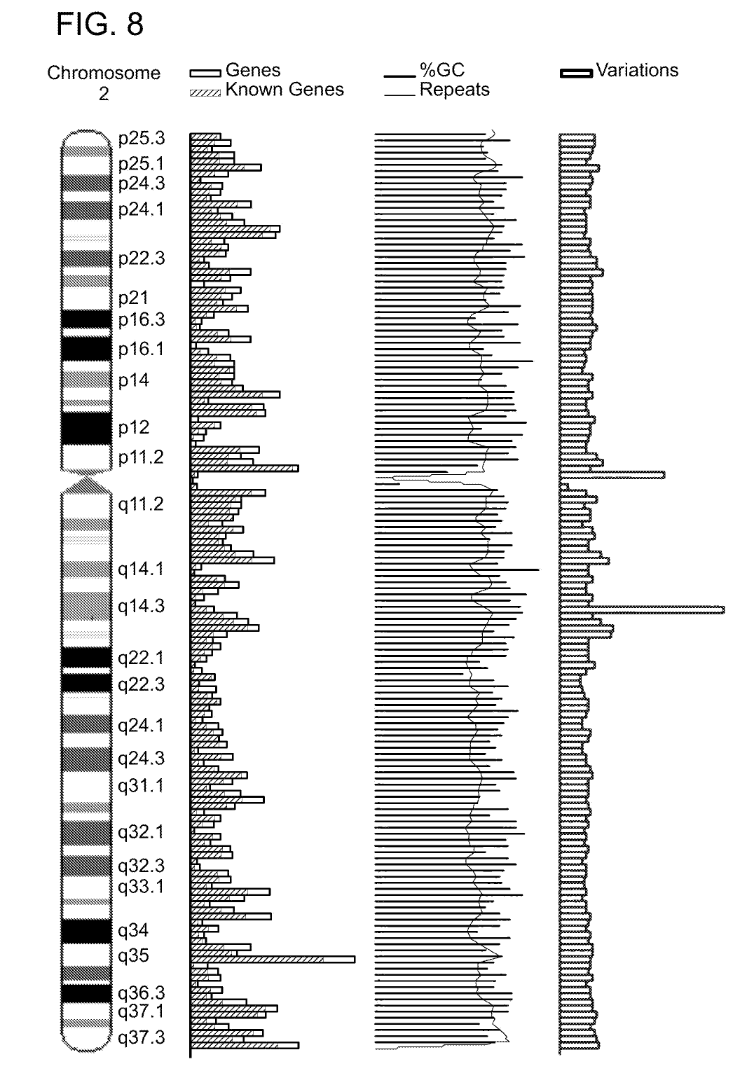

A Shared Segment Analysis revealed shared regions in affected individuals of unrelated families on chromosomes 2, 3, 5, and 15. The shared region size ranged from 7 Mb to 30 Mb. To further define the genetic mutation(s) with disease association in these regions, detailed mapping and sequencing was performed. FIGS. 5-7 show the analysis results for three families. In FIG. 5, candidate shared regions are shown on chromosome 5 and 2 (see narrower shaded bars on the chromosomes). Similarly, candidate regions on chromosomes, 15, 7, 5, 4, 3, and 2 are shown in FIG. 6. Similarly, a candidate region is shown on chromosome 2 in FIG. 7. FIG. 8 shows a more detailed map of chromosome 2, including an area of overlap between regions identified in different families.

In summary, this study revealed that known FSGS genes account for only a small fraction of FSGS occurrences in the patient cohort. Utilizing IBD detection analysis, highly consanguineous FSGS families provided clues for novel mechanisms of FSGS development and progression. Further, the identified regions are useful to identify novel gene(s) associated with FSGS that will be useful for more accurate diagnosis of FSGS in Saudi Arabia, the Gulf Region and worldwide.

It should be appreciated that assays for detecting the presence of one or more specific regions associated with an increased risk for FSGS may be developed and/or implemented based on standard molecular biology techniques known to one skilled in the art.

In some embodiments, the regions may be evaluated in patient samples using a panel of markers (e.g., 10-50, 50-100, 100-250, 250-500, or more markers, for example SNPs).

It should be appreciated that one or more analytical techniques described herein (including in the claims) may be used to evaluate the risk of FSGS (or of being an FSGS carrier in a specific family), for example, by focusing on one or more of the regions identified herein.

It also should be appreciated that aspects of the invention relate to identifying one or more genes and/or alleles associated with FSGS by further analyzing the chromosomal regions identified herein.

### Example 4

### Protein Expression Profiling of Familial Focal and Segmental Glomerulosclerosis (FSGS)

Some aspects of the invention relate to the identification of secreted proteins that can be used as serum biomarkers for FSGS. In order to identify protein biomarkers for FSGS, a protein profiling study was performed on a cohort of FSGS patients. Samples were obtained from the patient cohort described in Example 3 above. In this study, protein profiles were analyzed in sets of samples from normal individuals and patients, for example, by obtaining a test protein sample, for example, a serum protein sample, from an individual affected with FSGS, and a control protein sample from an individual not affected with FSGS. The proteins of the test sample and the control sample were then separated via 2D gel electrophoresis and the protein patterns obtained were analyzed for differentially expressed proteins.

Protein patterns in serum samples from six control and six FSGS affected individuals were analyzed using two-dimensional polyacrylamide gel electrophoresis (2-DE). Their protein expression profile was evaluated by computer-assisted image analysis (PDQUEST) and proteins were subsequently identified using matrix-assisted laser desorption/ionization mass spectrometry (MALDI-TOF-MS).

This global, unbiased protein expression profiling study led to the identification of overexpressed-in-FSGS proteins which may reveal worthwhile candidate biomarkers for FSGS.

Table 8 provides a list of desirable features of biomarkers for renal diagnosis.

TABLE 8
desirable features of biomarkers for renal diagnosis
Ideal biomarker for renal diagnosis

| Criterion | Desired Characteristics |
|---|---|
| Ease of | In urine, or in blood (stable) |
| measurement | |
| Sensitivity | Detected early |
| Specificity | Distinguish tubular from prerenal and glomerular |
| | injury |
| Predictive | Monitor progression/regression of injury and |
| ability | therapeutic response |

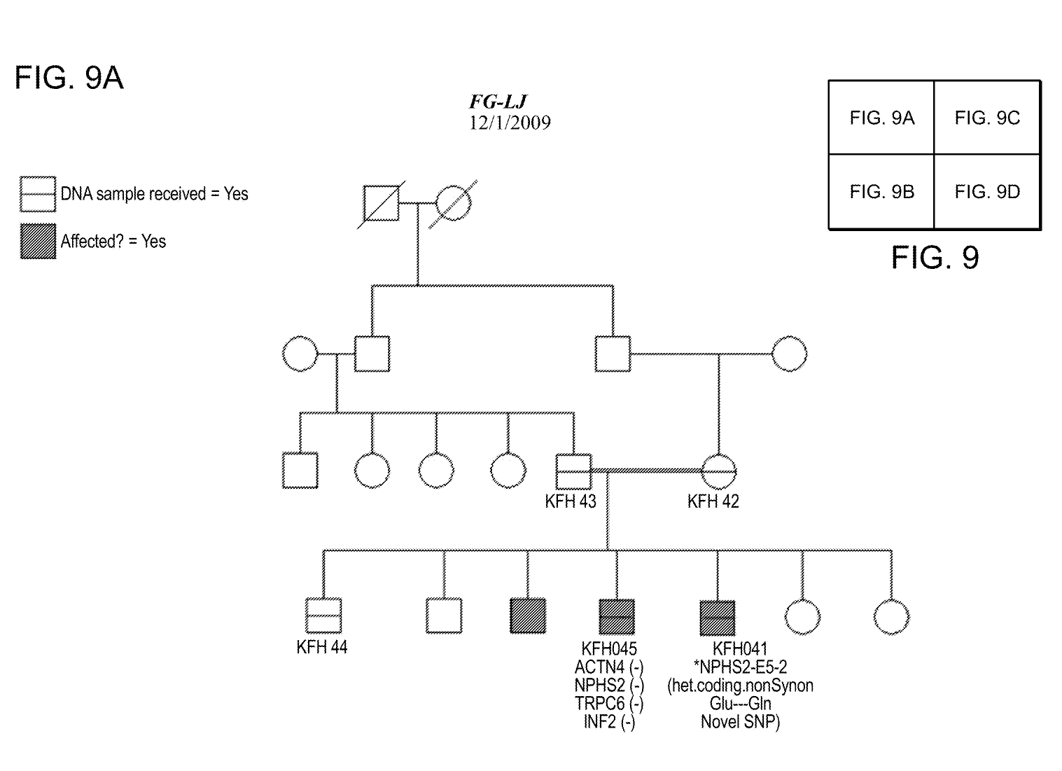

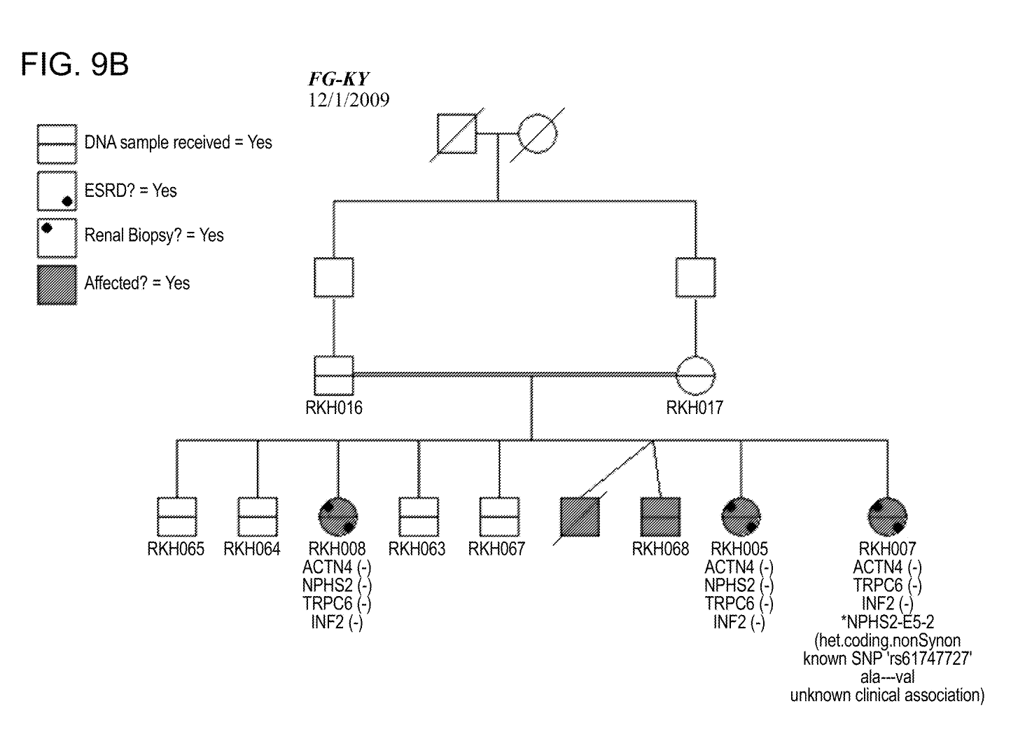

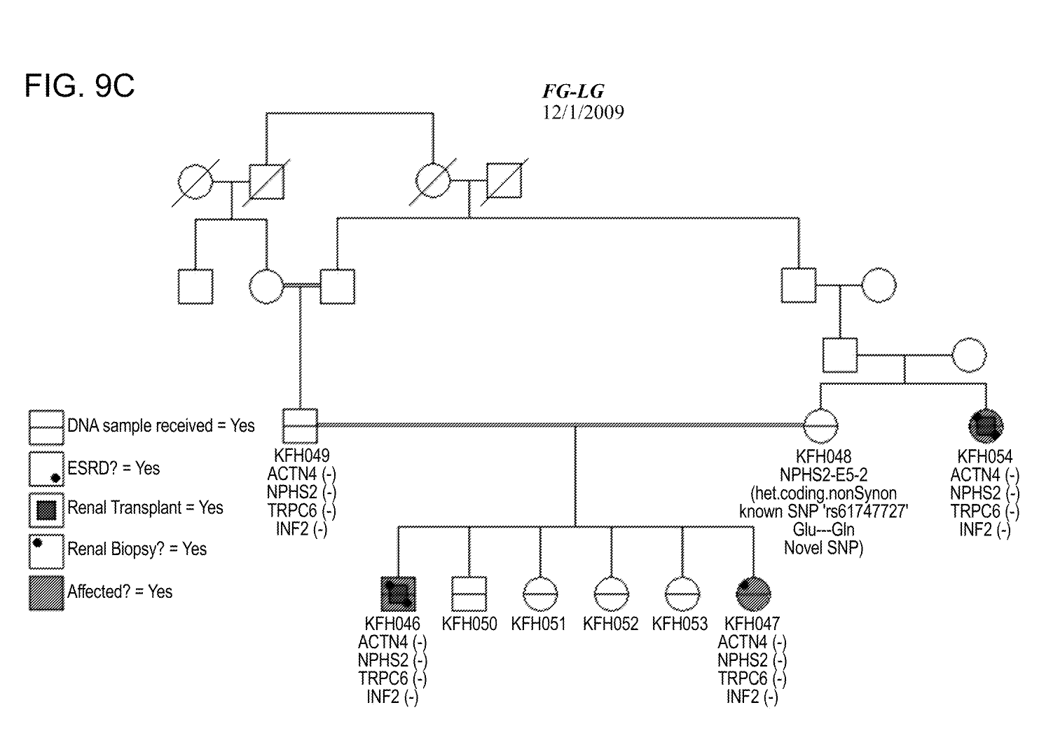

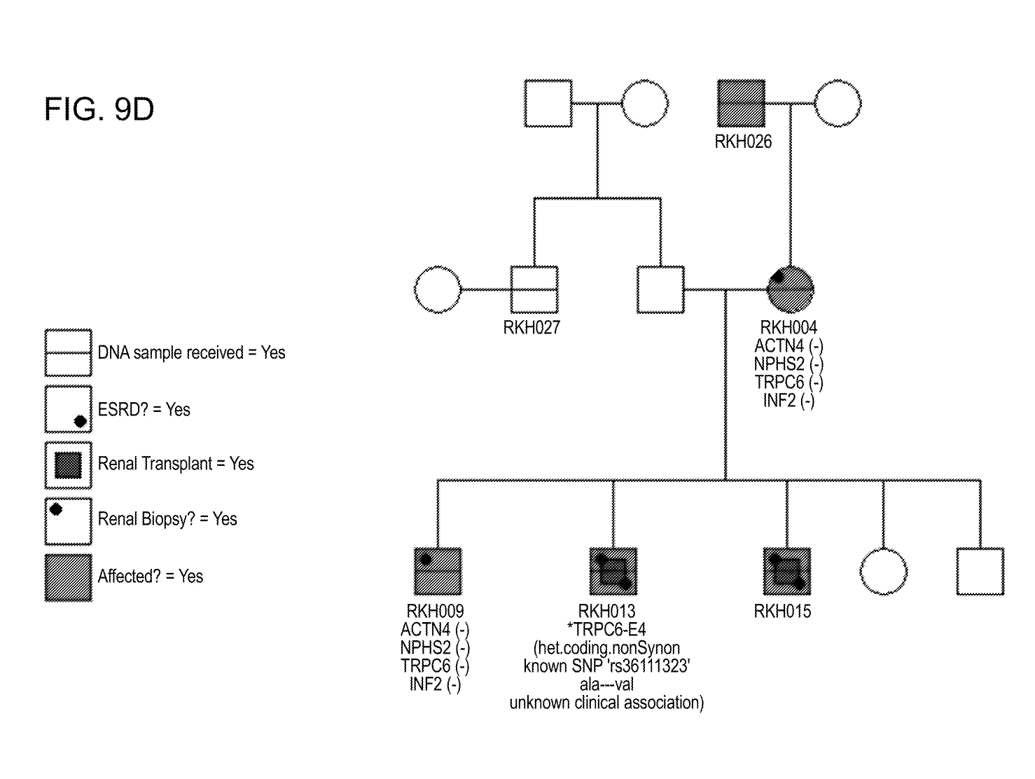

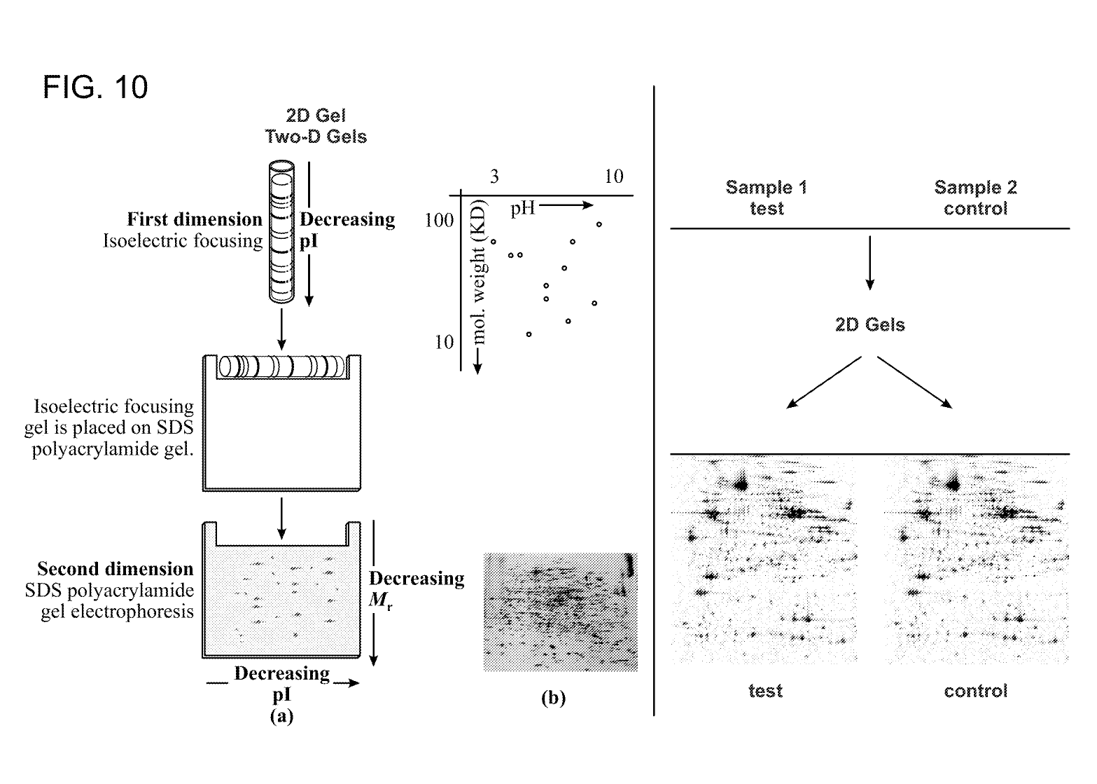

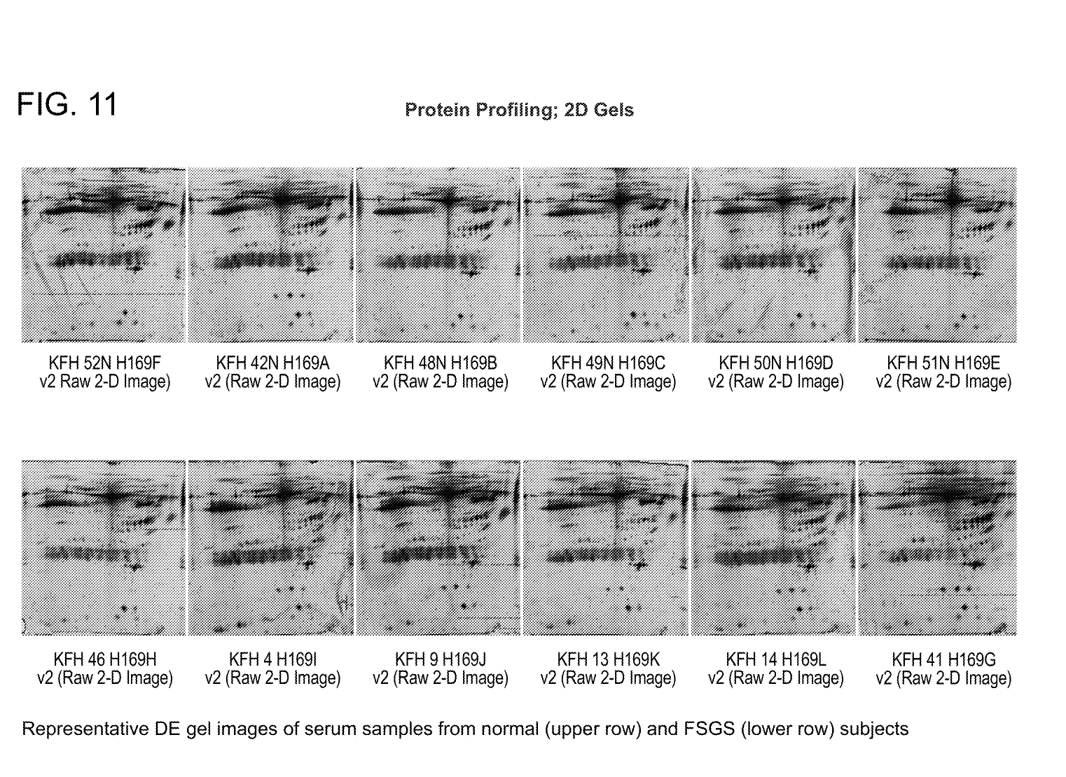

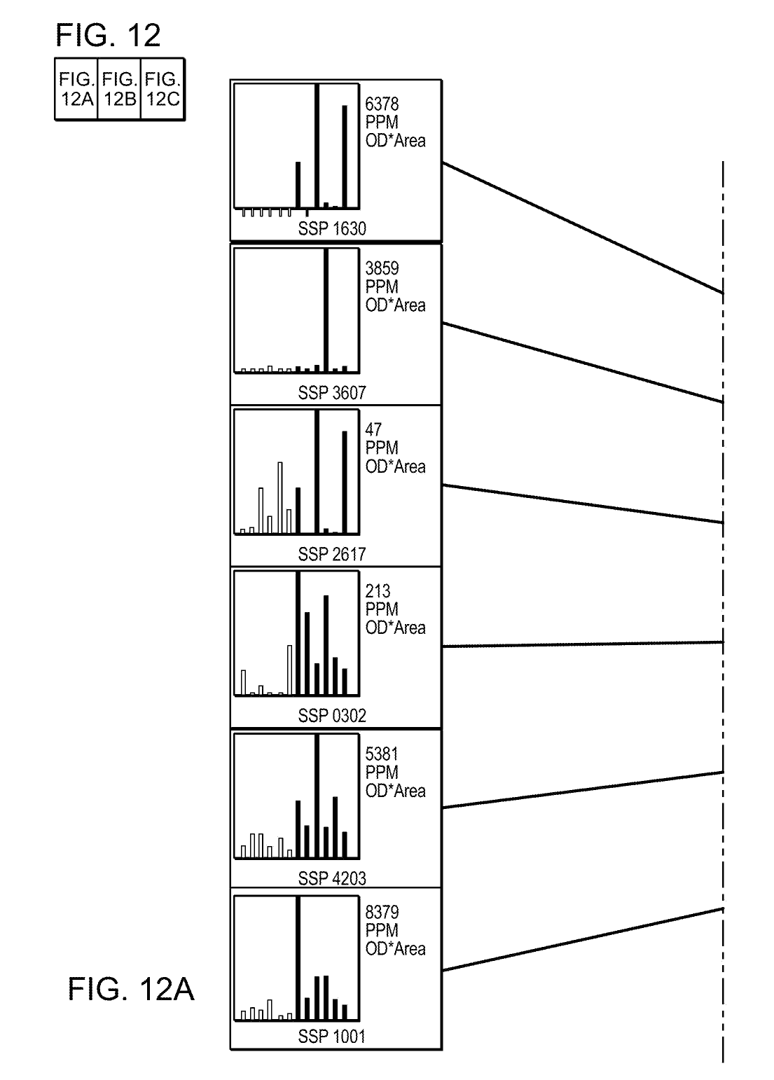

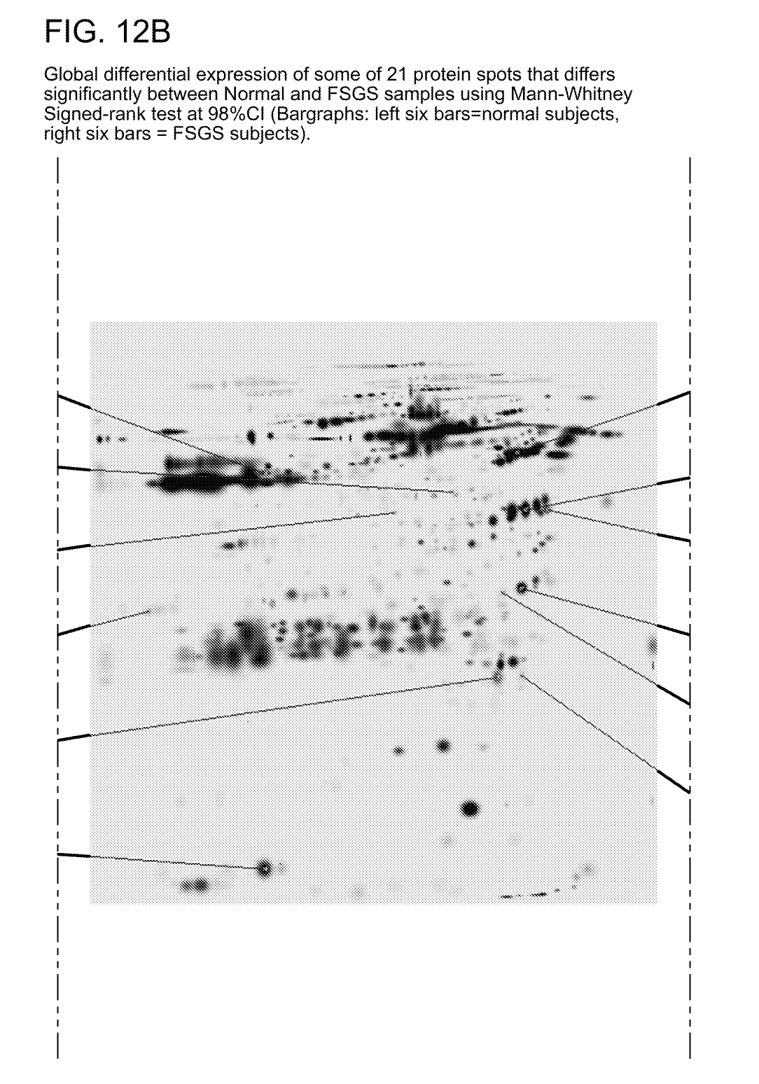

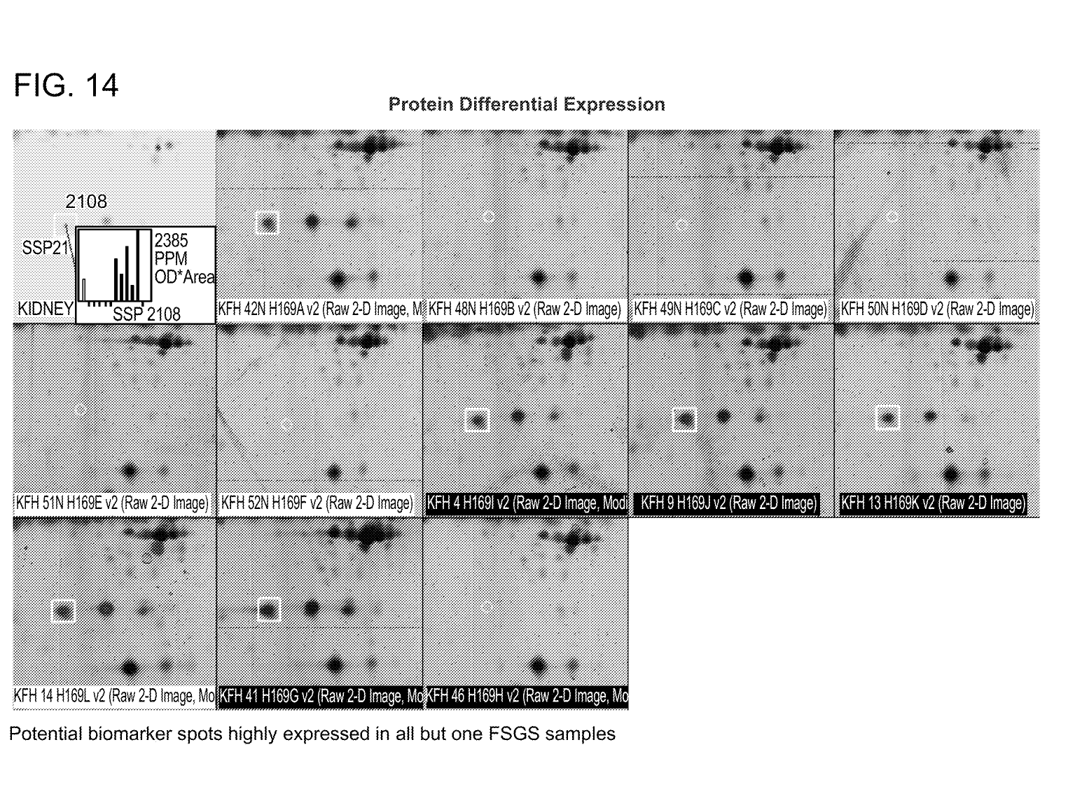

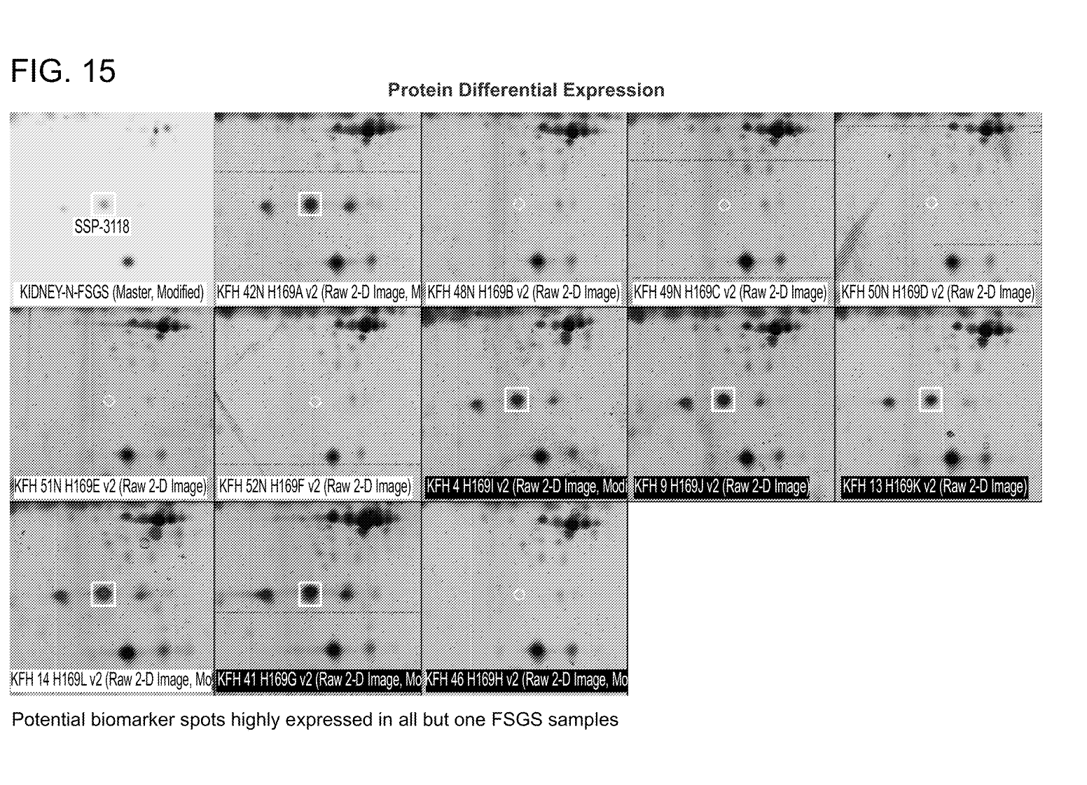

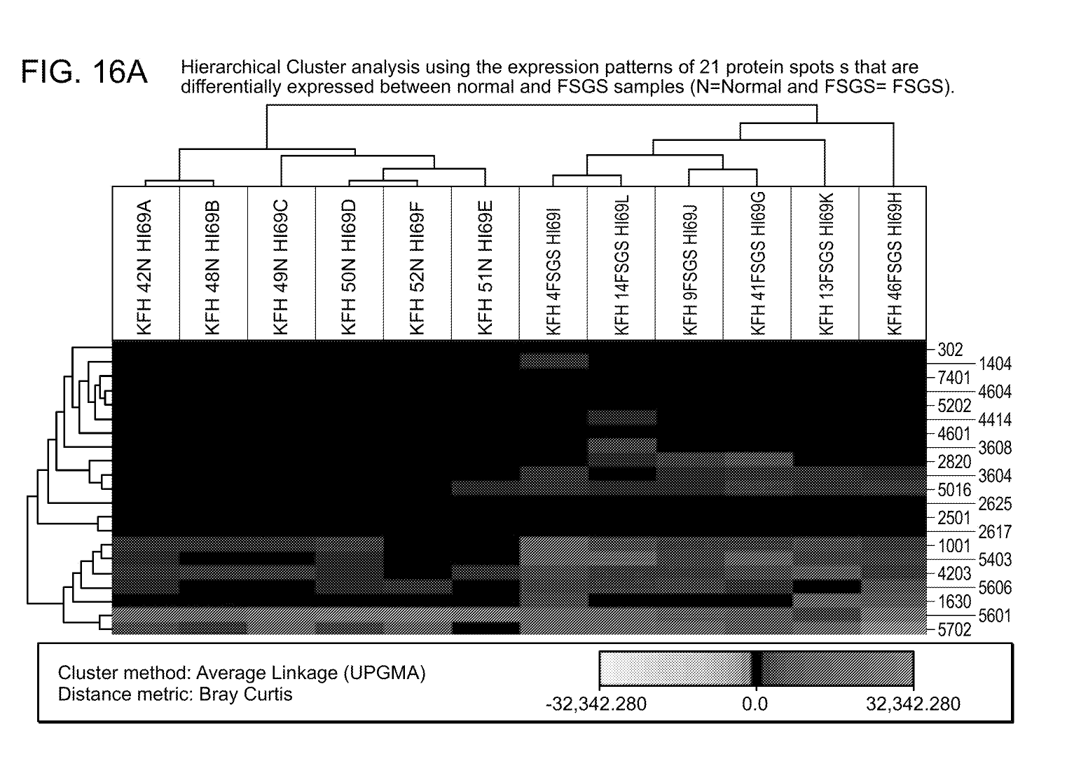

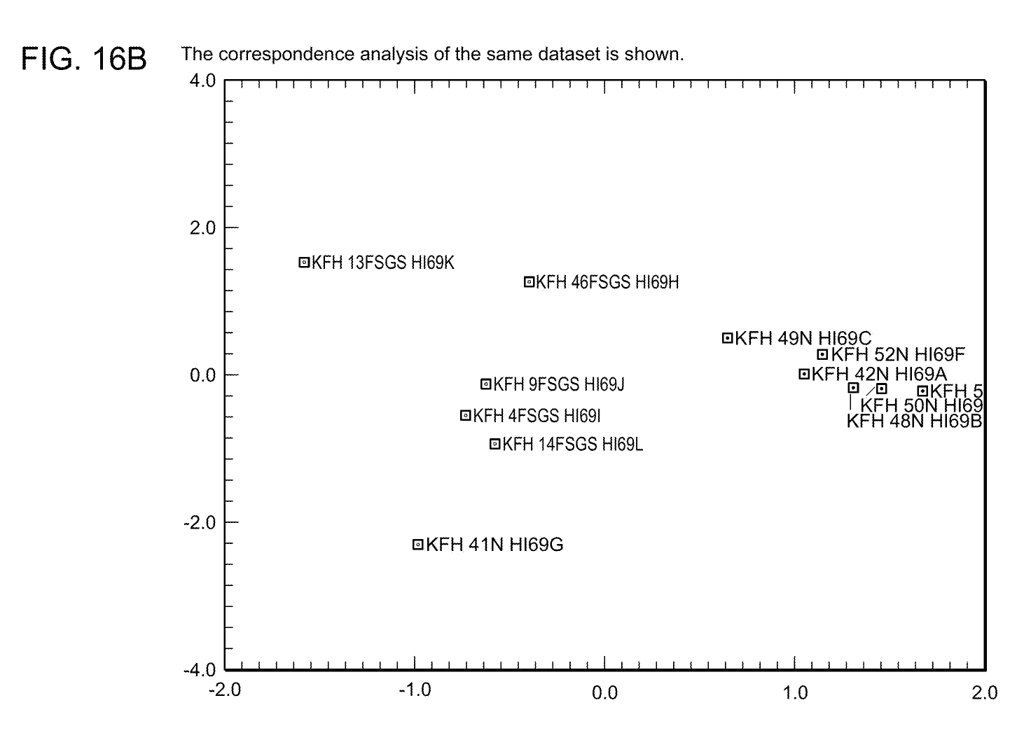

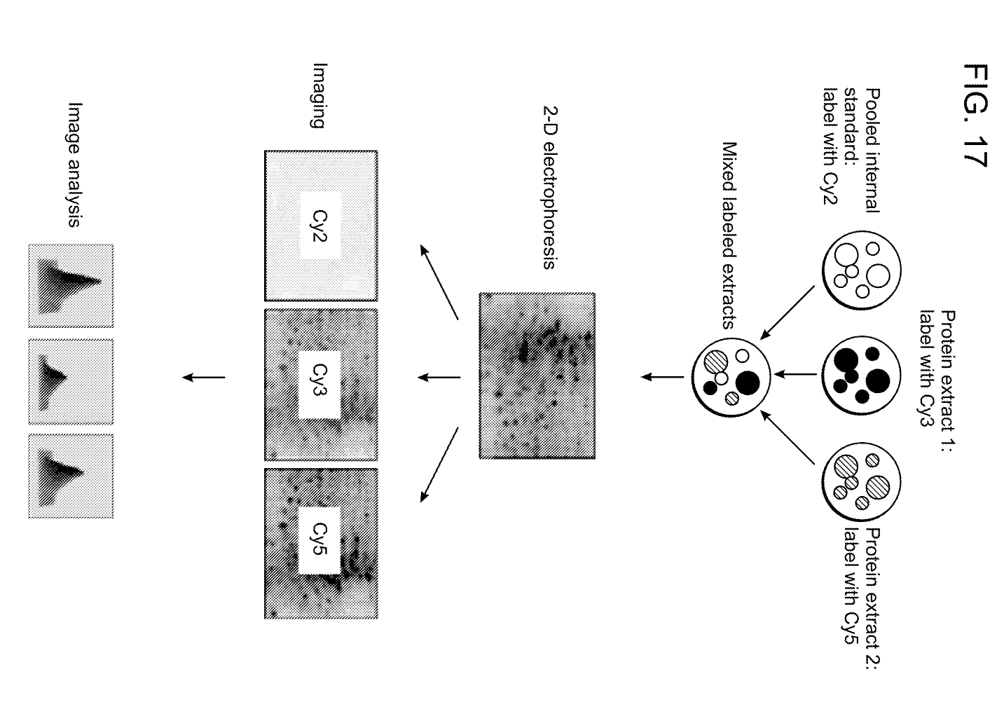

FIG. 9 shows the families that were analyzed in this study. FIG. 10 shows examples of 2D gels and results for test and control samples. FIG. 11 shows examples of protein profiling using 2D gels. FIGS. 12 and 13 show 21 protein spots that can be used according to aspects of the invention (e.g., as markers for FSGS). It should be appreciated that the presence of one or more of these markers in a sample may be evaluated using a 2D analysis, and/or using a ligand (e.g., an antibody) that specifically detects the presence of one or more of these markers in a patient sample. FIGS. 14 and 15 illustrate differential expression of certain protein markers. The figures also illustrate a protein of interest (see boxed spot). FIG. 16 illustrates a hierarchical cluster analysis using the expression patterns of 21 protein spots s that are differentially expressed between normal and FSGS samples (N in a sample name indicates normal sample; FSGS in a sample name indicates an FSGS sample). (A). The correspondence analysis of the same dataset is shown in (B). FIG. 17 illustrates a non-limiting technique for assaying samples for the presence of one or more FSGS-specific protein markers.

The protein expression data revealed that a distinct set of 21 protein spots were differentially expressed in FSGS vs. Control subjects. These differences were statistically significant between normal and FSGS samples using Mann-Whitney signed—Ranked Test and student's—t test (P<0.05). Eighteen of the 21 proteins were identified as serum/plasma specific proteins and were classified into two functional categories. Among the identified proteins under complement and coagulation cascades are Alpha 1 antitrypsin, beta-2 glycoprotein and alpha-1 microglobulin. The second group was classified as transport proteins and includes Transthyretin precursor (Prealbumin), Apolipoprotein E precursor, Apolipoprotein A IV precursor and Serotransferrin precursor. Other identified proteins are Zinc finger protein, involved in transcription regulation and Vitamin D binding protein precursor.

### REFERENCES

1. Mistry K, Ireland J H, Ng R C, Henderson J M, et al. Novel mutations in NPHP4 in a consanguineous family with histological findings of focal segmental glomerulosclerosis. Am J Kidney Dis 2007; 50: 855-864.
2. Thomas D B. Focal segmental glomerulosclerosis: a morphologic diagnosis in evolution. Archives of pathology & laboratory medicine 2009; 133: 217-223.
3. Denamur E, Bocquet N, Mougenot B, Da Silva F, et al. Mother-to-child transmitted WT1 splice-site mutation is responsible for distinct glomerular diseases. J Am Soc Nephrol 1999; 10: 2219-2223.
4. Boute N, Gribouval O, Roselli S, Benessy F, et al. NPHS2, encoding the glomerular protein podocin, is mutated in autosomal recessive steroid-resistant nephrotic syndrome. Nature genetics 2000; 24: 349-354.
5. Kaplan J M, Kim S H, North K N, Rennke H, et al. Mutations in ACTN4, encoding alpha-actinin-4, cause familial focal segmental glomerulosclerosis. Nature genetics 2000; 24: 251-256.
6. Winn M P, Conlon P J, Lynn K L, Farrington M K, et al. A mutation in the TRPC6 cation channel causes familial focal segmental glomerulosclerosis. Science (New York, N.Y. 2005; 308: 1801-1804.
7. Lowik M M, Groenen P J, Pronk I, Lilien M R, et al. Focal segmental glomerulosclerosis in a patient homozygous for a CD2AP mutation. Kidney international 2007; 72: 1198-1203.
8. Philippe A, Nevo F, Esquivel E L, Reklaityte D, et al. Nephrin mutations can cause childhood-onset steroid-resistant nephrotic syndrome. J Am Soc Nephrol 2008; 19: 1871-1878.
9. Brown E J, Schlondorff J S, Becker D J, Tsukaguchi H, et al. Mutations in the formin gene INF2 cause focal segmental glomerulosclerosis. Nature genetics 42: 72-76.
10. Pollak M R. The genetic basis of FSGS and steroid-resistant nephrosis. Seminars in nephrology 2003; 23: 141-146.
11. Pollak M R. Inherited podocytopathies: FSGS and nephrotic syndrome from a genetic viewpoint. J Am Soc Nephrol 2002; 13: 3016-3023.
12. Choi M, Scholl U I, Ji W, Liu T, et al. Genetic diagnosis by whole exome capture and massively parallel DNA sequencing. Proceedings of the National Academy of Sciences of the United States of America 2009; 106: 19096-19101.
13. Ng S B, Buckingham K J, Lee C, Bigham A W, et al. Exome sequencing identifies the cause of a mendelian disorder. Nature genetics 2010; 42: 30-35.
14. D'Agati V. Pathologic classification of focal segmental glomerulosclerosis. Seminars in nephrology 2003; 23: 117-134.
15. Henderson J M, Alexander M P, Pollak M R. Patients with ACTN4 mutations demonstrate distinctive features of glomerular injury. J Am Soc Nephrol 2009; 20: 961-968.
16. Hildebrandt F, Attanasio M, Otto E. Nephronophthisis: disease mechanisms of a ciliopathy. J Am Soc Nephrol 2009; 20: 23-35.
17. Saunier S, Calado J, Benessy F, Silbermann F, et al. Characterization of the NPHP1 locus: mutational mechanism involved in deletions in familial juvenile nephronophthisis. American journal of human genetics 2000; 66: 778-789.
18. Konrad M, Saunier S, Heidet L, Silbermann F, et al. Large homozygous deletions of the 2q13 region are a major cause of juvenile nephronophthisis. Human molecular genetics 1996; 5: 367-371.
19. Al Harbi N. Chronic renal failure in children in asir region of saudi arabia. Saudi J Kidney Dis Transpl 1997; 8: 294-297.
20. Al-Ghwery S, Al-Asmari A. Chronic Renal Failure among Children in Riyadh Military Hospital, Riyadh, Saudi Arabia. Saudi J Kidney Dis Transpl 2004; 15: 75-78.
21. Kari J A. Chronic renal failure in children in the Western area of saudi arabia. Saudi J Kidney Dis Transpl 2006; 17: 19-24.
22. Durbin R, Eddy S, Krogh A, Mitchison G. Biological sequence analysis: Probabilistic models of proteins and nucleic acids. Cambridge University Press, 1998.
23. Li H, Durbin R. Fast and accurate short read alignment with Burrows-Wheeler transform. Bioinformatics (Oxford, England) 2009; 25: 1754-1760.

All publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

1. A method comprising
determining a genotype or haplotype of the Nephrocystin-1 (NPHP1) genomic locus in a subject, and
if both alleles of the NPHP1 genomic locus comprise a loss of function mutation, identifying the subject as having or being predisposed to develop Focal Segmental Glomerulosclerosis (FSGS).

2. The method of claim 1, further comprising
obtaining a biological sample from the subject; and/or
choosing a course of treatment and/or administering a treatment appropriate for FSGS to the subject in order to prevent or delay development of FSGS in the subject.

3. The method of claim 1, further comprising performing an assay on the nucleic acid sample to determine the genotype or haplotype of the NPHP1 genomic locus.

4. The method of claim 1, wherein
the subject is homozygous for a loss of function mutation at the NPHP1 genomic locus;
the subject comprises a deletion of one or more of the following genes: MALL, NPHP1, LOC151009, LIMS3, RGPD8, RGPD6, or RGPD 5; and/or
the subject was identified as having proteinurea.

5. The method of claim 1, wherein the subject is an adult.

6. The method of claim 1, wherein the subject is not diagnosed or indicated to have nephronophthisis (NPH).

7. The method of claim 1, wherein the mutation is a deletion of a genomic region coding for the NPHP1 protein or a fragment thereof.

8. The method of claim 1, wherein the subject belongs to a family in which at least one member is or has been diagnosed with or affected by FSGS.

9. The method of claim 1, wherein the subject belongs to a family in which at least one member is or has been diagnosed with or affected by FSGS but no member of which has been diagnosed or affected with NPH.

10.-12. (canceled)

13. A method comprising
determining the genotype and/or haplotype of the NPHP1 genomic locus in a subject from a family with a history of FSGS,
comparing the genotype and/or haplotype to a genotype and/or haplotype of the NPHP1 genomic locus in a plurality of consanguineous subjects having FSGS, and
comparing the genotype and/or haplotype to a genotype and/or haplotype of the NPHP1 genomic locus in a plurality of consanguineous subjects not having FSGS, wherein
(i) if the genotype and/or haplotype of the subject comprises a loss of function mutation at the NPHP1 genomic locus that is shared among the subjects having FSGS, then the subject is indicated to be predisposed to develop FSGS, or
(ii) if the genotype and/or haplotype of the subject does not comprise a loss of function mutation at the NPHP1 genomic locus, then the subject is indicated to not be predisposed to develop FSGS.

14. The method of claim 13 further comprising choosing a course of treatment or administering a treatment appropriate for FSGS to the subject predisposed to develop FSGS to prevent or delay development of FSGS in the subject.

15. The method of claim 13, wherein
the NPHP1 loss of function mutation is a deletion of the NPHP1 gene;
the subject is identified as having a deletion of one or more of the following genes: MALL, NPHP1, LOC151009, LIMS3, RGPD8, RGPD6, or RGPD 5;
the subject is identified as being homozygous for a deletion of one or more of the following genes: MALL, NPHP1, LOC151009, LIMS3, RGPD8, RGPD6, or RGPD 5; and/or
the determining is before the onset of FSGS in the subject.

16.-18. (canceled)

19. A method comprising
determining the genotype and/or haplotype of the NPHP1 genomic locus of a male subject,
determining the genotype and/or haplotype of the NPHP1 genomic locus a female subject, and
if both genotypes and/or haplotypes share a loss of function mutation at the NPHP1 genomic locus, identifying their potential progeny as being at an increased risk to have a genotype predisposing the carrier to develop FSGS.

20. The method of claim 19, wherein the male subject and/or the female subject are from a family with a history of FSGS.

21. The method of claim 19, wherein the male subject and/or the female subject has a deletion of one or more of the following genes: MALL, NPHP1, LOC151009, LIMS3, RGPD8, RGPD6, or RGPD 5.

22. A method comprising
(a) analyzing proteins contained in a serum sample obtained from a subject from a family in which at least one member was or is affected by FSGS, wherein the subject has a deletion of both alleles of one or more of the following genes: MALL, NPHP1, LOC151009, LIMS3, RGPD8, RGPD6, or RGPD 5
(b) comparing the proteins contained in the serum sample of (a) to proteins contained in a serum sample from a consanguineous subject that does not have a deletion of both alleles of one or more of the following genes: MALL, NPHP1, LOC151009, LIMS3, RGPD8, RGPD6, or RGPD 5, wherein
(i) if a protein is contained in the serum sample obtained from the subject having the deletion but not in the serum sample from the subject not having the deletion, then the protein is identified as an FSGS-specific serum protein.

23. The method of claim 22, further comprising
obtaining the serum sample of (a) and/or of (b); and/or
performing an analytical assay to determine the levels of the proteins in the serum sample under (a) and/or (b), optionally, wherein the analytical assay is a 2D protein gel electrophoresis analysis.

24.-25. (canceled)

26. A method comprising
obtaining a biological sample from a subject,
determining the level of a first FSGS-specific serum protein in the sample,
comparing the level of the first protein to a reference level indicative of an average risk for FSGS, and
identifying the subject as having or being predisposed to FSGS, if the level of the first protein is statistically different than the reference level; or
identifying the subject as having or being predisposed to FSGS if the level of the first protein is statistically similar to the reference level.

27. (canceled)

28. The method of claim 26, wherein
the biological sample is a serum sample;
the first protein is a protein shown in FIG. 12, 13, 14, or 15;
the first protein is a member of a complement and/or coagulation cascade, a transport protein, or a zinc finger protein;
the first protein is selected from a group of proteins including alpha 1 antitrypsin, beta-2 glycoprotein, alpha-1 microglobulin, transthyretin, or a precursor thereof, apolipoprotein E, or a precursor thereof, apolipoprotein A IV, or a precursor thereof, serotransferrin, or a precursor thereof, and Vitamin D binding protein, or a precursor thereof; and/or
the level of the first protein is detected using an antibody assay.

29.-31. (canceled)

32. The method of claim 26, wherein the level of the first protein is detected using an antibody assay, an ELISA, or a Western Blot.

33. (canceled)
